(19) United States
(12) Patent Application Publication
(10) Pub. No.: US 2004/0029266 A1
(43) Pub. Date: Feb. 12, 2004

(54) Cell and tissue culture device

(76) Inventor: Emilio Barbera-Guillem, Powell, OH (US)

Correspondence Address: SEAN M. CASEY CO., LPA, P.O. Box 710, New Albany, OH, 43054-0710, US

(21) Appl. No.: 10/216,554

(22) Filed: Aug. 9, 2002

(51) Int. Cl. C12M 3/00

(52) U.S. Cl. 435/297.5; 435/304.1

(57) ABSTRACT

A cell culture device (100, 830) incorporating confronting planar anterior and posterior shells or walls (110, 140, 834, 836) that are joined about peripheral edges to define a media reservoir or cistern (170, 850). At least one of the shells and walls and edges is optionally formed with an aperture or respirator (180, 873). At least one fluid transfer port (220, 870) with a resealable elastomeric septum (230, 872) compatible for use with a small needle or needleless connector or pipetter tip (T, T′) is preferably formed in least one of the shells and walls (110, 140, 834, 836) and edges and that is in fluid communication with the media reservoir or cistern or chamber (170, 850). The device (100, 830) also includes at least one gas valvule (320, 875) that is formed in one or more of the shells and walls (110, 140, 834, 836) and edges and is in fluid communication with the media reservoir (170, 850) to vent gas from and supply gas to the reservoir (170, 850). The at least one gas valvule (320, 875) is preferably hydrophobic and is configured to pass only sterile air and to prevent liquid flow. In various embodiments, the preferred cell culture device (100, 830) that minimizes non-media containing headspace and that defines an internal surface (115, 145) having an area that bounds an internal volume whereby the ratio between the volume and the surface area is approximately between 100 microliters per square centimeter and 1000 microliters per square centimeter.

### TECHNICAL FIELD

This invention relates to a device adapted for use in maintaining and culturing biological cells in a medium. More specifically, the invention relates to an apparatus adapted to maintain and propagate prokaryotic, eukaryotic, hybrid, and artificial cells in a scientific research, laboratory, or clinical setting.

### BACKGROUND OF THE INVENTION

In the last few decades, the biological sciences have exploded in what has often been called the molecular revolution. A particular emphasis of modern biology is molecular biology, which is the study of the molecular building blocks and products of cells and sub-cellular structures and the relationships of those individual molecules to each other. Molecular biology encompasses such diverse fields of study as genetics, immunology, microbiology, cell biology, cell signaling, protein biochemistry, and a multitude of others. While molecular biology continues to focus on progressively and more discretely defined subject matter, the field is often hampered by problems associated with the ability to maintain and to propagate biological cells.

In much the same way that the diverse array of life as we know it can be placed into discrete classes, such biological cells of interest to molecular biology can be classified in broad terms as either prokaryotes or eukaryotes. Prokaryotes, a classification that includes principally archaebacteria and bacteria, are often referred to by those with skill in the art as simply bacteria. These prokaryotes, or bacteria, are usually single cells substantially capable of living free of associations with other cells. Such cells reproduce asexually, most often by binary fission. It is estimated that only a very small percentage of the bacteria that exist in nature can currently be grown or cultured in a laboratory, perhaps less than one percent. However, many of those bacteria, or prokaryotes, that can be grown in a lab tend to be relatively easy to grow and to propagate so long as the basic nutritional requirements of the cells of interest are supplied. Bacteria are also inclined to be hardy and resistant to environmental stresses such as transient peaks and troughs of nutrient availability, sub and supra optimal temperatures, harsh chemical agents present in the environment, and other less than desirable variations in environmental parameters.

Such bacteria have a significant impact on human existence and culture. For example, bacteria can cause disease in humans, crops, and livestock. Some bacteria naturally produce clinically desirable antibiotics and various therapeutic agents, while still other bacteria may be engineered to produce commercially valuable vaccines, insulin, growth hormones for humans and livestock, and products suitable for use in other economically and scientifically significant applications. Bacteria are both an ingredient in and a producer of food products such as yogurt and sauerkraut. Prokaryotes have even played a role in shaping the course of human history the black plague that redrew the geopolitical landscape of Europe was caused by a bacterium.

In spite of their relevance and importance to both human society in general and to molecular biology in particular, individuals with ordinary skill in the art generally reserve the terms cell culture and tissue culture for the maintenance and propagation of eukaryotic cells. Eukaryotes normally live in the multi-cellular arrangements such as plants, animals and mushrooms, although some eukaryotes such as the yeasts and the protozoa are usually single-celled. Eukaryotic cells can be capable of sexual reproduction, asexual reproduction, or both. Compared to the prokaryotes, eukaryotic cells tend to have more stringent nutritional needs and frequently require more precise and stable physical, biochemical, and thermal environments. When a eukaryotic cell is removed from an organism and placed into an appropriate nutrient medium, the cell will usually grow and divide by mitosis for only a few generations. Even if all environmental and nutritional conditions are ideal, such cells will lose viability in relatively short order. Such a eukaryotic cell culture is known to those skilled in the art as a primary cell culture. In contrast to primary cell cultures, some other eukaryotic cells and especially those cells derived from tumors or cancerous tissue will continue to grow and divide without unexpected or significant degradation or anomalous deterioration for as long as environmental and nutritional requirements are permissive for growth. Persons with skill in the art often refer to this type of eukaryotic cell culture as a cell line, permanent cell line, or as an immortalized cell line. Some of the cell lines studied today have been propagated in laboratories around the world for more than three decades.

A wide variety of eukaryotic cells and cell lines are of great interest and importance to modern molecular biology. Insecticides and herbicides, invaluable tools used to provide adequate food supplies for human populations, are typically engineered for and evaluated in eukaryotic cells from insects and plants. Eukaryotes help to feed people even more directly; virtually everything on a dinner plate is, was, or derived from a eukaryote. We not only consume but also are consumed by eukaryotes; malaria, an affliction that has killed more people throughout history than any other disease, is caused by a protozoan and is transmitted by an insect, both of which are eukaryotes. Eukaryotic cells can also help to cure diseases. Studies of both hereditary and communicable diseases often make extensive use of plant, animal, and human cells, all of which are eukaryotic cells. Vaccines and other therapeutics are normally tested in primary cell cultures and in immortalized cell lines long before they are evaluated in clinical trials. Some eukaryotes have even more direct clinical applications, antibiotics such as streptomycin and penicillin are natural products of eukaryotic cells. Many areas of eukaryotic cell investigation have the potential to provide enormous benefit to humanity. Examples of such areas of investigation include the interaction of human cells with pathogens, the cellular response to toxins, the development of treatments and cures for cancers and tumors, the regulation of the immune system, and the details of cell to cell signaling. Comprehension of how a single cell may be triggered to proliferate and then differentiate into an entire tissue or organ could translate into powerful new treatments for cancers, organ and heart diseases, and many other human maladies.

While living cells are normally classified as either eukaryotes or prokaryotes, a virus is yet another general type of biological agent not considered to be a cell by many persons skilled in the art. The viruses are unable to grow or propagate on their own in a nutrient medium because they depend upon a host cell to provide the biochemical machinery required for propagation of the virus. Those skilled in the art often refer to these types of parasites as obligate intracellular parasites. Some viruses can infect a wide range of cells and cell types, for example, influenza can infect respiratory tract cells of various waterfowl, seals, pigs, and humans. Other viruses may infect only one or a few specific cell types in a single species of host. For illustrative purposes, the Human Immunodeficiency Virus (HIV) will infect the T cells and macrophages of humans and will normally not, with limited exceptions, infect any other cell from humans or any other species. Nearly every cell identified to date, whether prokaryotic or eukaryotic, is a host to at least one virus, making viruses one of the most well represented biological agents on the planet.

Some viruses cause disease, such as HIV and the influenza virus mentioned herein. Others have little or no effect on their host cell. Still others can be beneficial; for example, the variegation popular in many types of tulips is caused by infection of the tulip plant with a virus. As another example of the potential utility of viruses, the vaccinia and other viruses have been used as vehicles to deliver vaccine to plants and animals. For these and other medical and commercial reasons, viruses are also of great interest to modern molecular biology. While viruses cannot be cultured per se, viruses may be propagated in appropriate eukaryotic or prokaryotic host cells.

In addition to viruses, eukaryotes, and prokaryotes, there are other known biological agents that do not fit into this classification system. Prions, for example, are naked infectious proteins that cause such animal diseases as Bovine Spongiform Encephalopathy (B.S.E., Scrapie, or “Mad Cow Disease”) and the human diseases Kuru and Creutzfeldt-Jakob disease. It should be understood that, while useful, the herein-described eukaryotic/prokaryotic classification system is a construct of the human mind that is designed to categorize and organize a myriad of data collected over thousands of years of human culture and science. It is a descriptive rather than a prescriptive system. To illustrate this point, consider chloroplasts, a sub-cellular organelle found in many plant and algal cells. It is believed by many of those skilled in the art that chloroplasts are derived from an ancient, free-living prokaryote. Chloroplasts are semiautonomous organelles that have their own genetic information distinct from that of the host plant or algal cell and that govern much of their own reproduction via organelle division. The present classification system sees the question of whether eukaryotic plant and algal cells that possess such organelles be considered eukaryotes or prokaryotes. Perhaps such cells should be classified as quasi-prokaryotic.

Even if nature had not provided cells and biological agents that do not fit easily into extant classification systems, humankind certainly has. For example, bacterial genes are commonly expressed in plant and animal cells, and vice versa. As another example, cells such as hybridomas are engineered by fusing dissimilar cells into a resulting hybrid cell. Regardless of the true nature of a cell or biological agent, be it a prokaryote, a eukaryote, a hybrid of the two, or even a yet to be discovered cell type, cell culture should be understood to be the deliberate growth and propagation of a particular cell or cell line of interest. This purpose may include any one or several of the following applications: 1) the harvest of the cells themselves to be used in some application, such as the growth and purification of the yeast cells that are combined with flour and water to make bread; 2) to reap some useful compound elaborated by the cells, such as the purification of human insulin from recombinant bacteria; 3) to harvest some cellular component such as membranes, antibodies, enzymes, and the like to be used for some subsequent application or purpose; 4) the evaluation or monitoring of some cellular process under various conditions, such as the response of cells to sudden changes in temperature or pH; 5) to assay, monitor, or study the cellular response to a pathogen, chemical, therapeutic, or other agent or condition; 6) to provide a sufficient number of appropriate cells to propagate a virus or other intracellular parasite; 7) any other circumstance in which cells, cellular products or biological agents are used, needed, desired, or involved.

There are undoubtedly far fewer eukaryotes in the world than there are viruses and bacteria. Nevertheless, eukaryotic cell biology occupies a significant portion of the focus of modern molecular biology because humans and their pets, livestock, and crops are all eukaryotes. Prokaryotes and viruses are also studied extensively, but frequently in the context of their impact on eukaryotes. With few exceptions, the raw material of modern molecular biology must be harvested from and evaluated in living cells, often in eukaryotic cells. Those with skill in the art have long recognized various problems central to this application in the field of molecular biology. For example, cells exquisitely adapted to life inside of and as a part of a living creature must be maintained and propagated with reasonably high yields in a laboratory or clinical setting: 1) without contamination; 2) without the loss of desirable traits; and 3) without the acquisition of undesirable traits.

Many specific additional issues arise from these and other core problems. First, a cell or cell line of interest often requires a substrate upon which to adhere during growth. Second, cells require regular exposure to or immersion in some form of a solid, liquid, gaseous, trans-phase, or multi-phase medium and or media that supplies nutrients and growth factors, and which media and or medium is also adapted to remove any potentially damaging waste products either by diluting them or during removal and replenishment of the medium and or media. A relatively small number of the cells of interest can be grown, but the geometry of the ratio of the surface area covered or immersed by the available medium to the volume of that medium makes higher cell yields cumbersome and unwieldy using present-day technology, methods, and equipment.

An additional issue in maintaining and growing cells is gas diffusion. During growth, cells must be exposed to precisely controlled and periodically replenished amounts of N₂, CO₂, O₂, and other gasses. The proper ratio of appropriate gasses can be either mechanically introduced into the nutrient medium on a regular basis or must passively diffuse into the nutrient medium via a phase boundary. Where the latter method is employed, the cell culture flask or multiple-well plate is usually placed into a substantially sealed compartment or container. This compartment or container is frequently referred to by those skilled in the art as an incubator, which is often maintained in a controlled environment selected to have a predetermined temperature, humidity, and gaseous composition.

Another and even more pervasive issue that continues to vex prior art devices is that of contamination, either with an undesirable cell such as a ubiquitous and hardy bacterium or fungus or with some other undesirable contaminant. Although the cells of interest must be exposed to an initial supply of media and gas and possibly to periodic replenishments of the same, it is critical to grow only the cells of interest without introducing undesirable contaminants. Since most living and non-living surfaces contain viruses, bacteria, fungi, and the like, it is often difficult to establish and maintain a cell culture without introducing undesirable contaminants. Such undesirable contaminants can cause a multitude of deleterious and costly effects. Contaminating cells can kill or injure the cells of interest by producing toxins or antibiotics. Undesirable cells can significantly reduce growth yields of the cells of interest by consuming nutrients and growth factors intended for the cells of interest. Even if the undesirable contaminants do not directly harm the cells of interest, they can contaminate any products being elaborated by the cells of interest. Such contamination can skew the results of any testing performed upon the cells of interest by producing unexpected or unknown substances or by producing markedly less than anticipated substances or a superabundance of anticipated substances.

For the purpose of explicating the field of the invention and background of the art, the terms cell(s), cell culture(s), culture(s), primary cell culture(s), cell line(s), and immortalized cell line(s) should be understood to refer to those cells of interest and their progeny that are maintained and propagated. Additionally, the terms culture, cell culture, and cell culturing may also be understood to refer to the process or technique of such cell maintenance and propagation for the reasons discussed herein or for any other purpose. Those having skill in the art may also use the term “tissue culture” in lieu of such terms, although this term is customarily restricted to the culture of eukaryotic cells derived from the tissue of higher, multi-cellular organisms. The cells of interest in cell culture are frequently but not necessarily eukaryotic cells. The terms undesirable cell and contaminant(s) should be understood to refer to unwanted or contaminating cells, viruses, prions, or other similarly undesirable or unwanted chemicals, compositions, elements, biological agents, components, or constituents. The terms medium and nutrient medium (media-plural) should be understood to refer to the solid, liquid, gaseous, trans-phase, or multi-phase medium that may supply nutrients, growth factors, trace elements, salts, buffering capacity, or any other element or component required or desirable to support the survival, growth, and or propagation of the cells and tissues of interest and their progeny.

The difficulties inherent in growing the cells of interest in a laboratory, research, or clinical setting may be better appreciated by considering one well-established and readily available cell culture technique. This approach to cell culture is to inoculate the cells into an appropriate medium and place the inoculated medium into a sterile vessel. If the sterile vessel includes a single compartment, then those with skill in the art customarily refer to it as a tissue culture flask, a cell culture flask, or a flask. While there are many exceptions to the general rule, the general rule is that there are two types of flasks: flasks for culturing eukaryotes and flasks for culturing prokaryotes. The former type of flask being preferably adapted to incorporate an atmosphere external to the media and or to exchange the gases either directly with the media contained in the flask or with such an atmosphere with an external replenishment source of gas. The latter type of flask is often referred to in its classical configuration by those skilled in the art as an Erlenmeyer flask and is most commonly adapted to culture prokaryotic cells and related tissues, materials, and substances with or without an atmosphere because such cells can be aerobic and also may be anaerobic such that they can be cultured without exposure to an external atmosphere for gas exchange. Examples of such cells include without limitation non-adherent tumor cells, bacteria, and hybridomas, to name a few. While either type flask can be adapted to support cell culture of cells that must attach to a surface to grow or which can grow unattached or in suspension, more customarily, the eukaryotic culture flasks have internal surfaces that are adapted or treated specifically for either attached or suspended cell growth applications. Most commonly, the prokaryotic cell culture flasks are adapted for unattached or suspended cell growth applications.

An illustration of a cell culture flask with some of these elements and others is found in U.S. Pat. No. 6,114,165 to Cai et al. The cell culture flask is typically a rectangular cube defining an interior space that is to be used for cell culture. Both the top and the bottom surfaces or dimensions of the cell culture flask preferably have substantially more surface area than any one of the four sides. In operation, the bottom of the cell culture flask is kept approximately horizontal and an opening to the cell culture flask is formed as a substantially vertical aperture located on one of the four sides of the flask. A cap is often removably affixed to the opening of the flask.

The sterile vessel may also have a plurality of compartments. In this arrangement, the vessel is then frequently referred to by persons skilled in the art as a multiple-well plate, with each compartment of the vessel defining a well formed in the plate. In this configuration there are four side walls that project upwardly from and substantially perpendicular to an approximately rectangular base member. Other walls project upwardly from the base member and attach to each other and to side walls to define the plurality of compartments or wells. A lid is often included in such devices, which lid is typically slightly longer and wider than the base member. The lid device functions by resting against and on top of the multiple-well plate, to enclose said multiple-well plate. An example of a multiple-well plate that incorporates some of these as well as other features is shown in U.S. Pat. No. 4,349,632 to Lyman et al.

In use, after cells are inoculated into the cell culture vessel, the cells of interest generally adhere to the bottom of the flask or well and propagate. While cells can be cultured in many types of flasks, cells do not grow or propagate equally well on all types of materials that are used to fabricate such culture flasks or vessels. As a result, considerable attention has been devoted to the investigation of various materials that have been developed and tested to ascertain their efficacy for the wide range of cell culture applications. Over the past many decades, many types of materials have become generally accepted by those skilled in the art as being preferred for use as flasks and for multiple-well plates. Such materials are most commonly selected from the group of materials that includes glass, ceramics, metals, thermoset and elastomer monomers and polymers, and polymeric thermoplastics including, for further purposes of illustration but not for purposes of limitation, thermoplastic materials selected from any of a variety of commercially available and suitable materials including acetal resins, delrin, fluorocarbons, polyesters, polyester elastomers, metallocenes, polyamides, nylon, polyvinyl chloride, polybutadienes, silicone resins, ABS (acrylonitrile, butadiene, styrene), polycarbonate, polypropylene, liquid crystal polymers, alloys and combinations and mixtures and composites thereof and reinforced alloys and combinations and mixtures and composites thereof.

While many configurations of cell culture devices, flasks, and vessels exist, most commonly, it is the bottom surface of the cell culture vessel where adherent-type cells are grown. Preferably, the bottom surface is kept in a substantially horizontal position during incubation and cell growth and is usually covered by a layer of the preferred nutrient medium. The medium is configured to supply necessary nutrients and growth factors to the cells. The rest of the internal volume of the flask or the well is, for eukaryotic culture applications, adapted to establish a volume space for the supply of gasses needed for growth and for the expulsion or diffusion of waste gasses that are the by-product of cell culture.

Those skilled in the art have come to investigate and understand many principles that guide the understanding of gas exchange during incubation between the external atmosphere or source of gasses, and the gasses or atmosphere contained in such a volume or head space in the flask, the media contained in the flask, and the cells that are either attached to a surface of the flask or that are unattached to any surface and suspended during growth in the media. Within and between the external atmosphere and the atmosphere contained in the head or volume space of the flask, the exchange and movement of gaseous or vaporous substances or the gasses is controlled by the random diffusive Brownian motion of the gas molecules, which is also affected by the temperature of the constituent gasses, and is further influenced by the kinetic energy of the gases which is parameterized by the molecular weights of the respective constituent gasses and many other parameters including, for example without limitation, the relative solubilities, concentrations, and partial pressures of such gasses.

With respect to the exchange and movement of vapors and gasses between the head or volume space in the flasks above the media, and the media, the rates of diffusion across the gas-liquid boundary at the surface of the media is a function of the preceding parameters as well as the solubilities, concentrations, temperature, and partial pressures of the gasses external to the media and those dissolved, absorbed, and otherwise present in and mixed with the media. The exchange and diffusion of gasses within the media is similarly affected by each of the preceding parameters, as well as by the formulation of the media, the type of cells being cultured, and by the potential energy inherent in the molecular structure of the media, which can further increase or decrease the kinetic or Brownian diffusion rates of gasses in the media and between the media and cells.

In sum, such gasses can efficiently and passively diffuse into the liquid medium because of the large surface area to volume ratio contemplated by the various cell culture devices, flasks, and vessels illustrated herein. In the majority of prior art devices, such gas exchange can only be accomplished with well-characterized and predictable results by establishing a large boundary interface between the liquid surface of the media and the head space or volume contained above to the surface. Even so, the vapors and gasses maintained in such head or volume space must be monitored and, depending upon the particularly application, removed and or replenished periodically so as to maintain preferably amounts of desired gases, such as diatomic oxygen and carbon dioxide, and or vapors, such as water.

The proper concentration of carbon dioxide can typically and preferably be about 5%, which is nearly twice that present in the Earth's ambient atmosphere, and which if deficient in an atmosphere proximate to a cell culture, can result in over diffusion of carbon dioxide out of the media and catastrophic over alkalinization of the cell culture media. Similarly, if the vapor pressure of water in or the relative humidity of the atmosphere proximate to the cell culture media falls to low, the media can quickly become catastrophically dehydrated. In contrast, over humidification and or failure to maintain proper water vapor pressures and temperatures of the culture can result in fog formation, which prevents visualization and imaging of the cell culture. This same over humidification issue can also result in condensation, which can create contamination pathways and or escape of culture materials from the flask or vessel.

Although this technology has been in use for some time to maintain and propagate cells, it is replete with the noted problems and other technical difficulties. Such past attempts at improving the art of cell culture remains severely hampered by many issues and problems and is although widely in use, very limited in the scope of its efficacy and applicability, and is generally unsuited for the purposes of the more highly refined, very precise, and high yield techniques, methods, and applications undertaken by modern biotechnologists. One significant restriction is that of limited growth yield.

For further example, when using previously described cell and tissue culture devices, flasks, vessels, and similar hardware and related techniques, the layer of the nutrient medium contained therein in which the cells are immersed must be relatively shallow, for example between about 3 to about 20 millimeters deep, for efficient gas diffusion and other reasons as explained herein. For added example, when using a standard T-75 cell culture flask, which can hold a total volume of about 75 milliliters, is preferred to use a media volume of about 25 milliliters for cell and tissue culture, which results in a media depth of about 3 millimeters when the flask is placed on its side as normally used for incubation.

With this configuration and arrangement of media and flask, unless the media is properly titrated with the proper concentrations of constituents for the particular application, and incubated under precisely controlled temperature, humidity, and related conditions, without carefully synchronously-controlled media replenishment, the cells being cultured may quickly exhaust the nutrients and growth factors supplied by this relatively small volume and depth of the liquid media. More significantly, toxic waste products that are a natural byproduct of cell growth and metabolism can quickly accumulate and kill or injure the desirable cells. In order to overcome these limitations, the old or spent liquid medium must regularly be removed and replaced with an approximately equivalent volume of fresh liquid. Each successive manipulation increases the chance of contaminating the cells of interest.

Compounding the problem of limited growth yield is the issue of available surface area. In molecular biology, the cells of interest are frequently derived from human, plant, or animal samples. Such a cell generally requires a substrate upon which to grow and is known to those skilled in the art as an adherent cell. These cells will often continue to grow and to divide until all of the available surface area provided by the flask or the well is occupied, a condition often known to those skilled in the art as confluence. After confluence, the cells will usually stop growing, a growth pattern in many instances referred to as contact inhibition. Such contact inhibited cells will often not grow, however, if the initial cell density is inadequate. That is, if there is too much surface area for the number of Cells in the inoculum, the cells will not readily propagate. In other words, the cells must be cultured initially in generally smaller flasks or wells and then, following confluence, the cells and their progeny must either be harvested immediately or be transferred to progressively larger flasks or wells. The cells may also be harvested from a given flask or well and then re-seeded into a plurality of new flasks or wells, a process known to those having ordinary skill in the art as splitting cells.

Before transferring cells to a new flask or well, adherent cells are removed from the substrate. This removal is typically by mechanical scraping or by chemical or enzymatic detachment of the cells from the substrate. Non-adherent cells are usually separated from the spent liquid medium by centrifugation before transfer to a new flask. This cycle of inoculation, harvest, and re-inoculation is performed serially in most applications until an adequate number of cells have been obtained. This procedure is cumbersome and labor intensive, is wasteful of supplies and media, and is prone to contamination because of the requisite frequency of manipulation.

Another shortcoming of this type of cell culture is that of efficient and effective gas exchange. As already noted herein, the requirement for a shallow layer of the nutrient medium for efficient gas exchange places a limit on the supply of nutrients and growth factors available to the cells, which also limits the growth yield of these cells. This shallow layer of nutrient medium is also prone to relatively rapid evaporation, since much cell culture is conducted at elevated temperatures of approximately 37 degrees Celsius. Even small amounts of evaporation can alter the concentration of, for example, metabolites, cofactors, salts, waste products, or growth factors in the nutrient medium, leading to non-permissive conditions and possibly to cell death. If the layer of nutrient medium is deepened to overcome these limitations, gas exchange is impeded and the cells may suffer from sub-optimal, non-permissive, or even lethal levels of CO₂, N₂, O₂, and other gasses.

This type of cell culture also results in a large volume of wasted space inside of the flask or the multiple-well plate, a space commonly known to those with skill in the art as headspace. This is inefficient because only a small fraction of the space occupied by the flasks or multiple-well plates is actually being used for cell culture, the rest is the headspace. Additionally, atmospheric levels of O₂ and CO₂, for example, are not conducive to growth of the cells and therefore the flask or the multiple-well plate must be placed unsealed into an artificially maintained atmosphere. The incubator is often used to establish this artificially maintained atmosphere. The flask or the multiple-well plate is not sealed when placed inside such an artificial atmosphere, as a seal would hinder the diffusion of fresh gasses into the headspace and diffusion of consumed gasses out of the headspace. The lack of an adequate seal increases the likelihood of contamination of the cells with undesirable cells.

Some attempts have been made to overcome the limitations of the herein-described technology by increasing the available surface area of the flasks and wells and by reducing the likelihood of contamination during manipulation. Among many of the already described elements of the prior art and others, O'Connell et al. in U.S. Pat. No. 5,272,084 teach the use of ridges or grooves in the substrate to increase the surface area available for cell culture. The proposed increase in available surface area should cause a proportional increase in growth yield, but the increase in surface area and yield is relatively modest because there are several problems attendant with the proposed approach to increased yields. Most prominently, the ridges or grooves influence the cells being cultured to propagate and grow unevenly with unpredictable results across the surface area. More specifically, depending upon the types of cells or tissues being cultured, the cells can be seen to aggregate in the valleys and be sparsely populated at the crests of the ridges or grooves. As to practical operational limitations, the cells are difficult to remove by either mechanical or chemical techniques for purposes of harvest, splitting, and or transfer. Even if chemical release techniques are used in combination with tapping and or scraping removal methods, the cells that have aggregated in the valleys still tend to be resistant to release. Additionally, the incorporation of the ridges and grooves in device such as the '084-type flask create optical aberrations in the walls of the vessel that preclude visual observations, analysis, and imaging. Even more importantly, such devices also create difficult to characterize and unpredictable results in terms growth rates and yields of anticipated and expected by products. These problems are compounded by the fact that there is little improvement made to the minimization of damage to cells during release and removal. In fact, those skilled in the art have reported that use of such devices as that disclosed in the '084 reference can result in destruction of up to approximately 30% or more of any cell or tissue culture that has been cultivated. Even if more refined chemical release and tapping techniques are employed to preserve the molecular integrity of the exterior cell walls, for example where the cellular surface receptors are of primary interest to the operator, much of the culture is lost because of the resistance to release of those portions of the culture that have aggregated in the valleys between the crests of the grooves and ridges of the proposed '084 apparatus.

The '165 patent to Cai et al., in addition to disclosing the elements already discussed herein, further teaches the use of a wide, oblong opening in lieu of the standard narrow, screw-top openings taught by the '084 patent and others, which suggests improved cell removal capabilities but which fails to address the noted pitfalls. Further to this type of proposed culture device, one of the elements taught by U.S. Pat. No. 5,523,236 to Nuzzo is the use of a hinged closure apparatus to be attached to the opening of the cell culture flask. The incorporation of either the wide, oblong opening or of the hinged apparatus may reduce the risk of contamination during a particular manipulation, but it does little to reduce the requisite frequency of manipulation that is an underlying cause of the contamination. Furthermore, the '165, '084 and '236 patents fail to address many of the other shortcomings or limitations of the prior art such as the need to lessen the excess of wasted volume manifest in the headspace.

Other attempts that have been made to overcome the difficulties of maintaining and propagating cells. Some of these attempts at improvement are now described for the purposes of illustration. U.S. Pat. No. 5,010,013 to Serkes et al., for example, discloses a roller bottle technique and device. One portion of the '013 patent instructs in the use of a cell culture flask that is substantially cylindrical. The cells and a relatively shallow layer of liquid medium are placed inside of the roller bottle flask, and the flask is then rotated about its longitudinal axis. Since the entire inner surface area of the roller bottle flask is exposed to the liquid medium at some frequency, this approach of the '013 reference can significantly increase the surface area available as a substrate for growth, which can also increase growth yields. However, attendant with the increase in surface of the Serkes et al. type devices is a drastic increase in the air or gas volume that is established in the head space with the cylinder. This essentially wasted volume does little to minimize the footprint of the device and in fact results in the requirement for larger incubation spaces and more lab bench space to accommodate the larger sizes contemplated by Serkes et al. and similarly configured devices.

The '013 reference, which teaches among other elements the use of corrugation or ridges to increase available surface area, has the same drawbacks noted herein in connection with similar technologies, including especially the difficulties imposed on the operator trying to mechanically scrape adherent cells from the walls of the roller bottle. Furthermore, the rate of rotation in such an arrangement has been noted by those skilled in the art to be critical to cell propagation. If the roller bottle turns too slowly, portions of the interior surface area will receive inadequate supplies of nutrients or even become dehydrated and the cells will be distressed and or die. If the roller bottle turns too rapidly, the shearing forces present in the resulting fluid media flow can physically distress the cells causing lysis and detachment of adherent cells from the substrate. Furthermore, the constant mixing of gas and liquid inside of the roller bottle may result in frothing or bubbling. Frothing can denature proteins associated with the cells, thereby killing or injuring the cells. Frothing can also denature proteins found in the liquid medium, thereby destroying the very cell products that are to be studied or used.

Another attempt to overcome the deficiencies of the prior art involves the use of semi-permeable or selectively permeable membranes to supply fresh gas or nutrients or to remove waste or desirable metabolic end products. This technology can be seen in, for example, U.S. Pat. No. 6,043,079 to Leighton and U.S. Pat. No. 6,329,195 B1 to Pfaller. The '079 patent teaches, among other things, the use of a membrane in which the cell culture is sealed and to which the cells of interest adhere. When a cell culture device so constructed is immersed in a nutrient medium, preferably a liquid medium, nutrients diffuse into the cell culture and waste products diffuse out of the cell culture. One of the components taught by the '195 patent is the use of an additional gas permeable, liquid impermeable membrane for the diffusion of fresh and waste gasses. The use of semi-permeable and selectively permeable membranes reduces or eliminates the requirement to frequently access the interior of such a sealed cell culture flask and more closely mimics the in vivo conditions preferred by some cell types. However, cells have widely disparate requirements for nutrients, cofactors, pH, gasses, and the like. A bath of nutrient medium appropriate for one cell or cell line may not support another cell line. Since the cell culture devices described by the '079 and '195 patents are immersed in or placed into contact with the pool of nutrient medium, this technology hinders or even precludes the simultaneous culture of cells or cell lines with different or incompatible needs. Furthermore, while this membranous device reduces the risk of contamination by repeated entry into the flask to supply fresh medium and to remove spent medium, this technology requires that the exterior of the cell culture flask to be handled aseptically too. A contaminant on the exterior of such a cell culture flask can contaminate the pool of nutrient medium that it contacts. The membrane described by the '079 and '195 references, as well as other references in the art is generally impermeable to such contaminating cells, but contaminating cells in the pool of nutrient medium can consume nutrients intended for the cells of interest and may elaborate potentially damaging or lethal products to which the membrane is permeable. This contamination can potentially damage the cells of interest or alter them such that they are less useful or useless for their intended purpose. The need to aseptically handle the exterior of such a cell culture flask places an additional burden on the operator. Furthermore, many of these cell culture devices do not appear to contemplate and are not readily adapted to the high yield cell culture needed in many biotechnology and molecular biology applications.

Some examples of the prior art appear in some respects to contemplate high density cell culture and even suggest some attempts that may avoid some of the shortcomings found in using semipermeable or selectively permeable membranes for purposes of gas exchange and replenishment. For example, international Patent Cooperation Treaty (PCT) Publication WO 00/56870, published Sep. 28, 2000 to Barbera-Guillem, (hereafter also referred to as “the '870 device”) teaches among other elements the use of two such membranes sealed to a plastic frame such that the membranes and frame define an interior chamber. In operation, a technician suspends cells of interest in a nutrient medium and then injects the cell suspension into the device via an access port. The cells adhere to the membrane and gas exchange with the cells takes place across the membranes. A technician may remove spent medium and add fresh medium as needed using a needle introduced through a resealable septum.

One of many significant limitations of the '870 device is the that it appears to establish an internal positive pressure as the operator or technician injects suspended cells or fresh medium into the device, which injection compresses the preexisting volume of air and builds pressure inside the sealed chamber. Without venting to release excess pressure, the interior chamber pressure may become sufficiently high to burst or rupture the membrane, thereby ruining the device, destroying the cells of interest as well as any potentially valuable or important cell products, as well as contaminating the surrounding environment. Even if the membrane remains intact, pressures slightly above atmospheric pressure may be sufficient to lyse or damage relatively fragile cells and components thereof.

In the best of all possible circumstances, it appears from the proposed '870 apparatus that the cells and tissues being cultured are under pressure above ambient atmospheric pressure. Thus, it is also further apparent that the contemplated membranes of the '870 could rupture with only minor abrasion in normal use or from impact with a sharp instrument or edge since the contents are under pressure and the membranes are described as being only thin polymeric films. For a number of reasons, it also further appears that the cells or tissues could be physically damaged during chemical release and removal or aspiration through the resealable septum taught in the '870 reference.

Initially, the shearing forces in the flowing liquid media encountered by the cells during withdrawal or aspiration through the proposed needle of the '870 reference, which in embodiments available from the assignee corporation can be as long as about 10 to 12 millimeters or more, when compounded with the unavoidable change in pressure, may have dire consequences—most notably breach of the cell walls causing complete lysis of the cells of interest. Next, it appears that the '870 device, when used in the dual confronting membrane configuration illustrated will experience collapse of the membranes against one another as the media is withdrawn, which thereby results in any cell culture contained therein, whether attached to the membranes or in suspension in the media, being crushed against the collapsing membranes.

Third, since the cells cultured in a device according to the '870 reference may, during infusions and aspirations, be exposed to a pressure that is much greater than and a vacuum that is much less than the ambient atmospheric pressure. Harvesting the cells through a needle lumen subjects the cells, whether it be the cells passing through the needle lumen or being left behind as media is withdrawn, to possibly harsh rapid pressurization and decompression. Even if various techniques are employed to mitigate such effects, such as incremental aspiration and injection of air to minimize decompressive effects, the cells may be exposed to repeated compression and decompression, which can shock the cells. Moreover, if air is injected periodically during withdrawal, such air must be sterile, which adds further complexity and added steps to the process.

The rapid decompression effect, also known to many people as “the bends,” is also experienced by individuals diving in water who rise too quickly after operating at depths below sea level and under corresponding pressures above ambient atmospheric sea level pressure. With more specific reference the device taught by the '870 and related references, the gases dissolved in the media and the cells contained in the '870, are under pressure. When the pressure of the media and the cells is reduced during removal, the dissolved gases expand and bubble, which creates the bends or rapid decompression effect that destroys the cells.

The rapid decompression effects noted herein may be exacerbated as to those cells that are actually removed. Those skilled in the art of fluid flow dynamics can appreciate that when any particles of fluid are accelerated to have a velocity that is different from its initial or nominal velocity prior to such acceleration, which velocity could be zero, then the particles experience a net drop in the pressure associated with the volume proximate to the particles. More generally and with respect to the device of the '870 reference, the cells that may be withdrawn through the needle lumen will experience an additional pressure drop while being accelerated and withdrawn through the needle lumen. Thus, it can be further understood by those knowledgeable in the related arts that that compounded pressure drop experienced by such cells will only further induce breach of the walls of the cells. If this lethal combination of compounded pressure drops does not damage the cells moving through the needle lumen, then upon exiting the needle lumen, the reintroduction of what may be standard atmospheric sea level pressure may finally rupture the cell structure, which may have at least been weakened by the earlier rapid decompression effects. Accordingly, those skilled in the art of cell culture techniques and devices may be able to appreciate that such devices, like that contemplated by the '870 and related references, do little to improve the state of the art of cell culture devices and methods.

Additionally, devices like those described by the '870 reference, can require technicians to remove adherent cells from their substrate, the membranes of the '870 device, before harvest by either chemical or mechanical methods. To accomplish this mechanically with the '870 device, the technician must physically break into the device apart or cut the membranes to expose the cells of interest, and then to scrape the membranes. This process increases the risk of contamination of the cells of interest, can physically damage the cells, and may expose the technician to possibly biologically harmful cells or by products. These risks are more pronounced because the '870 device can operate during incubation under a pressure above atmospheric such that the membranes may rupture in an uncontrolled manner due to the sudden pressure release experienced when scoring or cutting the membranes. In fact, the very type of small diameter needle contemplated for use with inoculation and or injection of cells and media into and aspiration of same from the device of the '870 can present an enormous threat of puncture of the membranes and subsequent rupture, especially in the hands of an untrained technician. Further, even if chemical release means are employed to harvest cells from the membranes without breaking the device or cutting the membranes, the '870 device appears to be very limited in its capability to withstand the extraordinary loads and forces encountered during centrifugation subsequent to cell release, since the membranes of the '870 device are necessarily thin and may rupture when subjected to such forces.

As with many other prior art attempts, the devices described in the PCT WO 00/56870 reference to Barbera-Guillem also suffer from other shortcomings, including the inability to mitigate dehydration in environments having unsuitable or less than optimum humidity control capabilities. In fact, the device contemplated by Barbera-Guillem et al. must receive fluid replenishment as often as or nearly as often as other prior art devices so that the proper or desirable cell culture hydration can be maintained. Each instance wherein replenishment is required is an additional instance when infections can be inadvertently introduced or when other similarly debilitating mishaps can occur.

There are several additional examples of the prior art that are related to the herein-captioned reference PCT WO 00/56870. For example, international Patent Cooperation Treaty Publications WO 02/41969, WO 02/42419, and WO 02/42421 all published on May 30, 2002 to Barbera-Guillem et al. These patents teach various combinations and variations of the PCT WO 00/56870 including the use of magnetic sheets for magnetic separation, methods of adhering and removing such magnetic sheets, the use of a single rather than two semi or selectively permeable membranes, and other elements and techniques. The devices taught by these patents are substantially similar to the PCT WO 00/56870 Publication and they each share the very same limitations and shortcomings discussed herein. As such, these devices can increase the time spent by technicians on cell culture, can increase the risk of contamination of entire batches of cell cultures, can be unnecessarily wasteful of money and other resources, and can generally increase the burdens of high density cell culture.

Still another attempt to address certain of the limitations of the prior art involves the use of biological or bio-mimetic substrates to support the growth and propagation of cells. These substrates may be derived from or closely mimic the extra-cellular compounds upon which cells may grow in vivo. Such substrates include, for example, collagen, elastin, cartilage, and cellulose, all common components of the extra-cellular matrix of higher animals or plants. As an example of this type of technology, one of the teachings of U.S. Pat. No. 6,312,952 to Hicks is the use of cartilage and type I collagen arranged in layers to form a support matrix sub structure bathed in a liquid nutrient medium and upon which the cells of interest are cultured. Another teaching of the '952 patent is the use of a composite cell culture, the simultaneous culture of more than one cell type; in this case chondrocytes are provided as an accessory cell that, under the right conditions, can promote the growth and proliferation of certain cells of interest such as epithelial cells. The use of these types of substrates is reasonably well suited for certain applications, such as the culture of epithelial cells in a pseudo-epithelial arrangement to be used for transplantation or for the promotion of wound healing in vivo. However, these substrates may be poorly adapted to support the growth of other cells of interest, or may be no more useful or effective at promoting the growth of those cells than the glass, plastic, or other commonly used substrates. Furthermore, the production of the support matrix substructure can be labor intensive, time consuming, and may be expensive, depending upon the availability of the substrate of interest and the purity required for the desired application. What is more, this technology is not well suited for those applications that require high yield, high density cell culture.

There have been attempts to overcome the shortcomings of the prior art that do contemplate high density, high yield cell culture. An example of such an attempt is the use of three-dimensional arrays of microfibers as a substrate to support the culture of cells. The microfiber array is encased in a substantially sealed container, such container defining a microfiber-enclosing cavity, an entry port, and an exit port. Nutrient medium enters the container by way of the entry port, washes over and immerses the microfibers, and then leaves the container via the exit port. In this arrangement, the microfibers provide significantly more surface area than in any Of the other prior art, the constant flow of nutrient medium provides a steady supply of fresh medium and removes spent medium, and high cell yields are possible. An example of such a device is disclosed in U.S. Pat. No. 4,546,083 to Meyers et al., which teaches some of these elements as well as others.

While the microfiber arrays may increase the growth yield of the cells of interest, these fibers may be difficult and expensive to obtain or manufacture, compared to the previously described art. A plurality of variables is considered by the operator in choosing a microfiber array optimized for a particular cell of interest. These variables include, for example, available fiber surface area, fiber dimension, priming volume, flow properties, and others. A microfiber array optimized for one cell or cell line may be sub-optimal or even non-permissive for another cell or cell line, requiring additional time to optimize a new microfiber array and additional money to purchase such a microfiber array. Regardless of the particular microfiber array used in an application, the very nature of the three-dimensional array may preclude mechanical harvesting of the cells and may interfere with other harvesting methods. Additionally, dedicated machinery is used to provide a reservoir of fresh medium, to pump the medium into the entry port of the microfiber-enclosing cavity, and to collect the medium from the exit port of the microfiber-enclosing cavity. Such dedicated machinery can be expensive, difficult to maintain, and unlike the previously described incubator may not be useful for the culture of other cells or in other applications. Furthermore, any of the various pumps, valves, reservoirs, and the like used in this technology that come into contact with the nutrient medium must be sterilized before each use and must be maintained in such a way as to prevent contamination during use. This sterilization places an additional burden on the operator and maybe difficult to establish and to maintain, particularly in valves, interior compartments, and other similarly inaccessible or non-obvious locations.

A related but distinct attempt to surmount the limitations of the prior art are those devices sometimes referred to by those skilled in the art as chemostats. In one arrangement, such devices pump nutrient medium from a reservoir into a well or wells containing the cells to be cultured. As a given well fills, the nutrient medium flows into the next well and so on until the nutrient medium fills the final well and flows into a second reservoir configured to capture spent medium. The chemostat may also be configured with a single well. Such chemostats may be suited for the study of a particular culture over an extended period of time. Chemostats are, however, not well adapted to high yield cell culture and are also subject to many of the same limitations as the microfiber array technology. To the extent that a given well of the chemostat is substantially similar to the cell culture flask or to the well of the multiple well plate, the chemostat is also subject to many of the same limitations of available surface area, effective gas diffusion, and the like. Furthermore, where the chemostat is configured with a plurality of wells, each well of the chemostat receives nutrient medium from the same reservoir. This technology is therefore not well suited for the simultaneous culture of cells or cell lines with different nutritional needs. This technology is also not suited for the simultaneous culture of different cells or cell lines with the same or similar nutritional requirements, since cells from a given well may spill into a subsequent well and formed an undesirable mixed culture. An example of a chemostat that discloses, for example, some of these elements is found in U.S. Pat. No. 6,271,027 B1 to Sarem et al.

The need remains for a cell culture apparatus that both provides sufficient surface area in a manageable volume without the need for cumbersome manipulations and allows for the efficient exchange of gas, nutrients, and waste without excessive risk of contamination or the use of excessive headspace. While many of the prior art devices were aimed to improve the art of such devices, none has achieved the optimized and effective capabilities and widespread compatibility of the instant invention. The present invention meets the herein described and other needs without adding any complexity, inefficiencies, or significant costs to implementation in existing applications and environments. The various embodiments of the present invention disclosed are readily adapted for preferable ease of manufacture, low fabrication and setup costs, effectiveness of operation, and for wide compatibility with extant cell culture technologies.

### SUMMARY OF INVENTION

In its most general configuration, the present invention advances the state of the art with a variety of new capabilities and overcomes many of the shortcomings of prior devices in new and novel ways. In one of the many preferable configurations, a cell culture device includes substantially planar anterior and posterior shells or walls or faces that are arranged in a substantially confronting relationship, and which are joined by respective opposing dextral and sinistral laterally opposed longitudinal edges, and opposing superior and inferior peripheral lateral edges. The shells or walls or faces and the edges together define a media reservoir or chamber or cistern. Optionally, at least one of the anterior and posterior walls or faces or shells and the edges are preferably formed with at least one circumfluent periphery that defines at least one optional respirator aperture. The optional at least one respirator is formed from a gas permeable film or membrane that seals the optional at least one respirator aperture about the periphery. The device also further incorporates at least one fluid transfer port that is formed in least one of the shells and edges and that is in fluid communication with the media reservoir or chamber or cistern. At least one gas valvule is also formed in at least one of the shells and edges of the cell culture device and is also in fluid communication with the media reservoir. The valvule is adapted to equalize positive and vacuum pressure within the cistern to ambient atmospheric pressure as fluid is communicated through the at least one port. The at least one gas valvule is preferably hydrophobic and can be adapted to prevent liquid flow there through either by selection of an appropriately capable material, or by incorporating an additional valving device, or by including a combination thereof.

The cell culture device also is further adapted so that the surface area of the optional respirator membrane or film is approximately between 1% and 10% of the surface area of the media reservoir, and more preferably approximately between 1.5% and 5%. The optional membrane or film can also be formed from a sheet material to have a thickness approximately between 0.09 and 0.14 millimeters.

Preferably, the media reservoir is adapted to receive approximately between 20 milliliters and 140 milliliters of a fluid mixture, and more preferably at least about 25 milliliters. In various embodiments and depending upon the desired uses and applications, the preferred cell culture device is adapted with the joint that is formed between the respective lateral, superior, and inferior peripheral edges being a releasably or permanently hermetically sealed joint.

In various modifications and configurations, the cell culture device may have the media reservoir being formed with a lateral dimension between the laterally opposing longitudinal edges of approximately between 6.5 centimeters and 9 centimeters, a longitudinal dimension between opposing superior and inferior lateral edges of approximately between 11 and 13 centimeters, and a dimension between interior surfaces of the anterior and posterior shells or walls or faces of approximately between 2 millimeters and 6 millimeters.

In any of the preferred arrangements and configurations, the media reservoir is preferably defined by an internal surface area of the shells or faces or walls and edges that bounds an internal volume whereby the ratio between the volume and the surface area is approximately between 100 microliters per square centimeter and 1000 microliters per square centimeter, or more and depending upon the desired application. Moreover, and again depending upon the proposed applications and uses, the cell and tissue culture device can be adapted to have the media reservoir being defined by a plurality of internal surfaces of the shells and walls and faces and edges wherein substantially all of the surfaces are adapted to support growth of cells. In the alternative, only selected portions of the internal surfaces can be so adapted whereby certain other portions are adapted to inhibit such cell and or tissue growth.

The anterior and posterior shells can be, in various arrangements, be formed from a substantially transparent thermoplastic material. In alternative configurations, the thermoplastic material can be modified with a pigment that is selected for its capability to filter photonic energy outside the range of between approximately 500 and 600 nanometers, and even more preferably between about 550 and 570 nanometers, so that the energy absorbed by the cells and tissues being cultured can be closely controlled, among other possible uses and purposes.

In still more alternative configurations, the cell culture device may have the at least one fluid transfer port and or the gas valvule are adapted to communicate fluid (liquid and or gas) with the media reservoir through a siphon lock lumen formed with at least one fluid path that bends through at least one angle of approximately between 45 and 135 degrees of arc so as to equalize hydrostatic pressure against the port to minimize the possibility of leaks during use, handling, and related operations. In any of the preceding embodiments, the at least one fluid transfer port can also preferably incorporate a resealable elastomeric septum adapted to releasably receive a means to communicate a fluid through the port that can include needles of all types and various types of needleless connectors and lumens and various types of what are known to those skilled in the art as pipetter tips.

The contemplated at least one gas valvule incorporates a filtration element is also preferably adapted to pass only and or primarily gaseous atoms and molecules and to prevent the passage of particles having an average diametrical dimension of approximately between 0.1 and 0.3 microns. The filtration element or elements may also be formed from a material that is or that incorporates a hydrophobic material capable of minimizing and or eliminating the possibility that liquid will pass through the gas valvule. In alternative modifications, the filtration element can be formed from an assembly of at least 2 layers with a first layer being adapted to prevent the passage of particles having an average diametral dimension of at least between about 80 to 120 microns, and a second layer being adapted to prevent the passage of particles having an average diametral dimension of at least between about 0.1 to 0.3 microns. In yet other alternative arrangements, the filtration element is constructed or formed from a hybrid filter medium having a filtration property wherein the size of the particles that are filtered and or passed changes across a cross-section of the filter medium such that at a first exterior surface the medium is adapted to prevent the passage of particles having an average diametral dimension of at least between about 80 to 120 microns, and whereby at a second opposite exterior surface the medium is adapted to prevent the passage of particles having an average diametral dimension of at least between about 0.1 to 0.3 microns.

In still more optional variations of any of the preceding embodiments, modifications, and alternative configurations, the cell culture device may be modified wherein the anterior and posterior shells, faces, or walls and the laterally opposed peripheral longitudinal edges are adapted to form a body, which can be formed in any number of ways including extrusion methods, to have a superior body edge and an inferior body edge, wherein the superior and inferior peripheral lateral edges are further formed on respective superior and inferior end manifolds adapted to be respectively engaged with the superior and inferior body edges to further define the cistern. The manifolds and the body can be further formed with various lumens and channels that can be configured to infuse and aspirate fluids, including gases and liquids to and from the cistern, chamber, or reservoir of the cell and tissue culture device.

Other configurations of the instant cell and tissue culture device further contemplate an insulative and protective container that is formed with at least one interior cavity sized and adapted to receive one or more cell culture devices. The container can be further adapted to incorporate a means for controlling the temperature of the device, which means can include a power source, a thermo electric heat semiconductor pump, various control electronics, and heat conducting plates that can, in operation, control the temperature of the cell and tissue culture device for purposes of cooling and or warming the contents thereof.

These variations, modifications, and alterations of the various preferred embodiments may be used either alone or in combination with one another as can be better understood by those with skill in the art with reference to the following detailed description of the preferred embodiments and the accompanying figures and drawings.

### BRIEF DESCRIPTION OF THE DRAWINGS

Without limiting the scope of the present invention as claimed herein and reference is now made to the drawings and figures, wherein like reference numerals, and like numerals with primes, across the several drawings, figures, and views refer to identical, corresponding, or equivalent elements, components, features, and parts. In the various figures and drawings, as needed for purposes of better describing the aspects of the instant invention, various reference symbols and letters are used to identify significant features, dimensions, objects, and arrangements of elements described herein and in connection with the several figures and illustrations.

FIG. 1 is an elevated isometric view, not to scale, of the cell culture flask according to the principles of the instant invention;

FIG. 2 is an elevated, rotated, exploded, and reduced scale isometric view of the device of FIG. 1;

FIG. 3 is an elevated, rotated, and exploded isometric view, in reduced scale, of the device of FIG. 2;

FIG. 4 is a detail view, rotated and in enlarged scale, of a portion of the device of FIG. 2, with certain structure removed for purposes of illustration;

FIG. 5 is a detail view, rotated and in enlarged scale, of a portion of the device of FIG. 3, with certain structure removed for clarity;

FIG. 6 is a detail view, rotated and in enlarged scale, of a portion of the device of FIG. 2, with certain structure removed for purposes of illustration;

FIG. 7 is a detail view, rotated and in enlarged scale, of a portion of the device of FIG. 3, with certain structure removed for clarity;

FIG. 8 is a detail view, rotated and in reduced scale, of the superior portion of the anterior side of the cell culture flask of FIG. 1;

FIG. 9 is a detail view, rotated and in reduced scale, of the device of FIG. 8 in operation;

FIG. 10 is a detail view, rotated and in reduced scale, of the superior portion of the anterior side of the cell culture flask of FIG. 1 reflecting an alternative configuration;

FIGS. 11 and 12 are detail views of the device of FIG. 10 in operation;

FIGS. 13 and 14 are perspective views, rotated and in reduced scale, of the cell culture flask of FIG. 1 and reflecting modified embodiments;

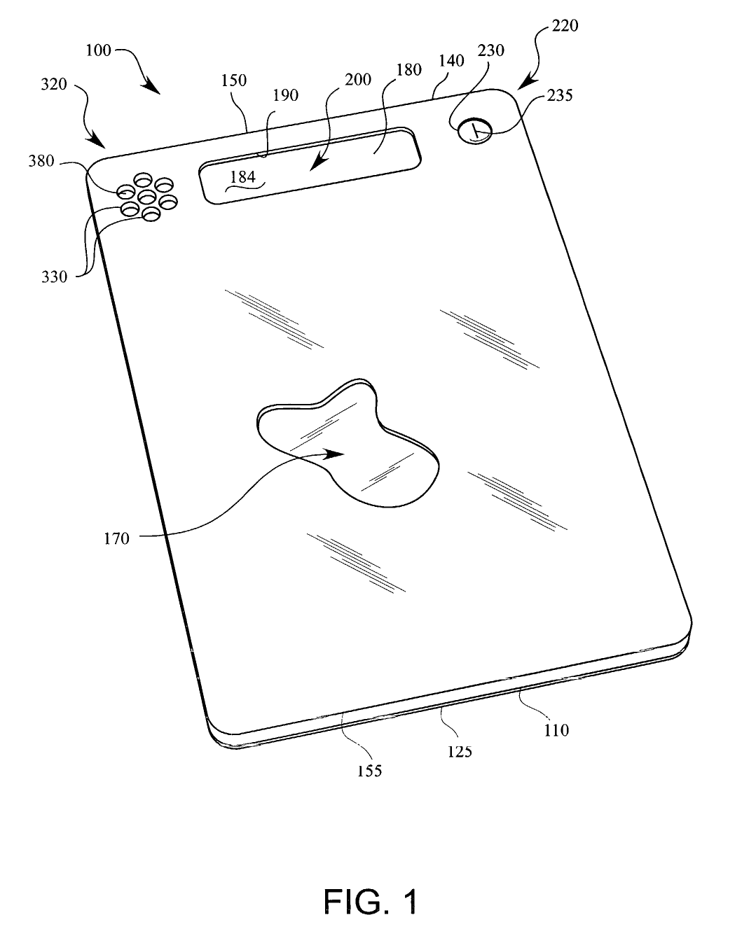

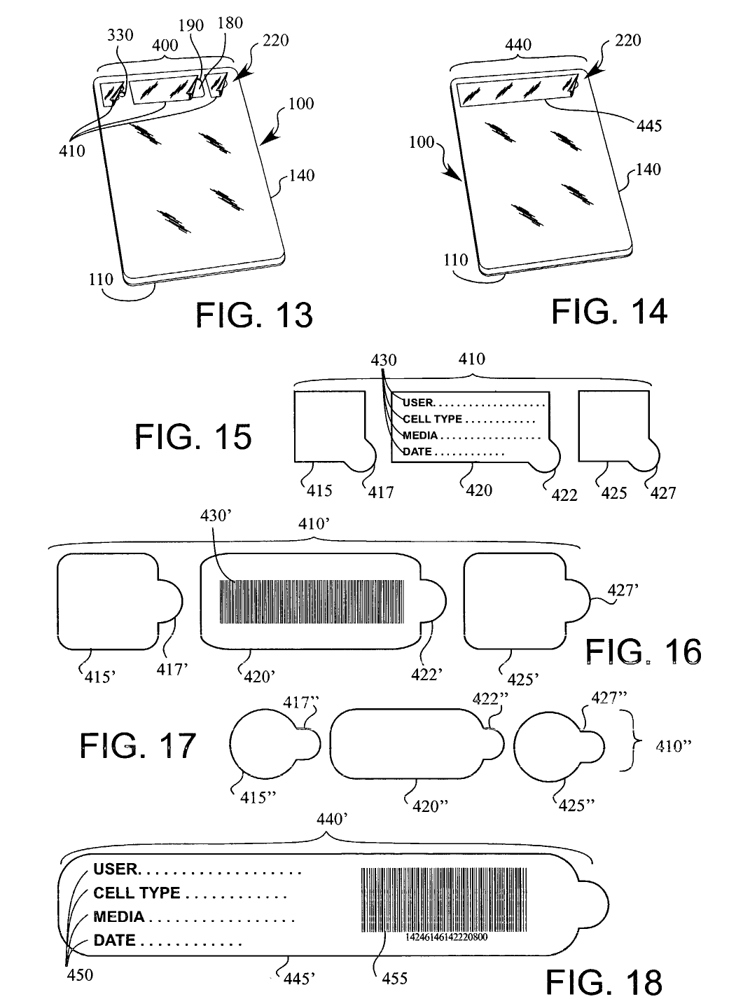

FIGS. 1 and 17, are plan detail views, rotated and in enlarged scale, of selected elements of FIG. 13 and with various structure removed for purposes of illustration;

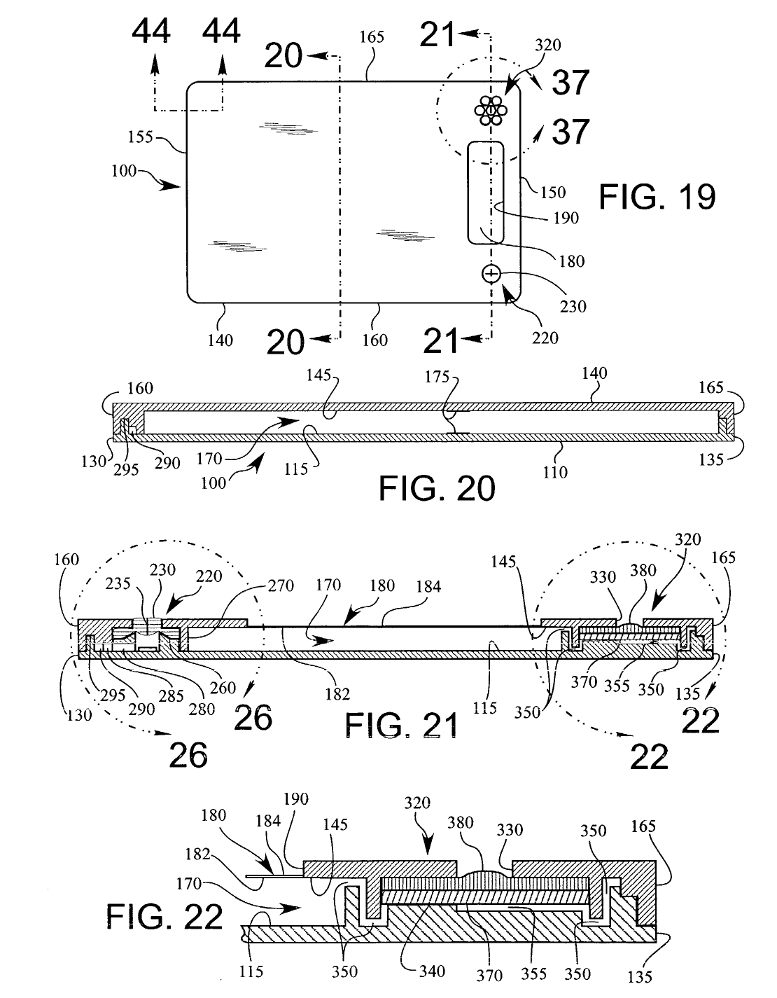

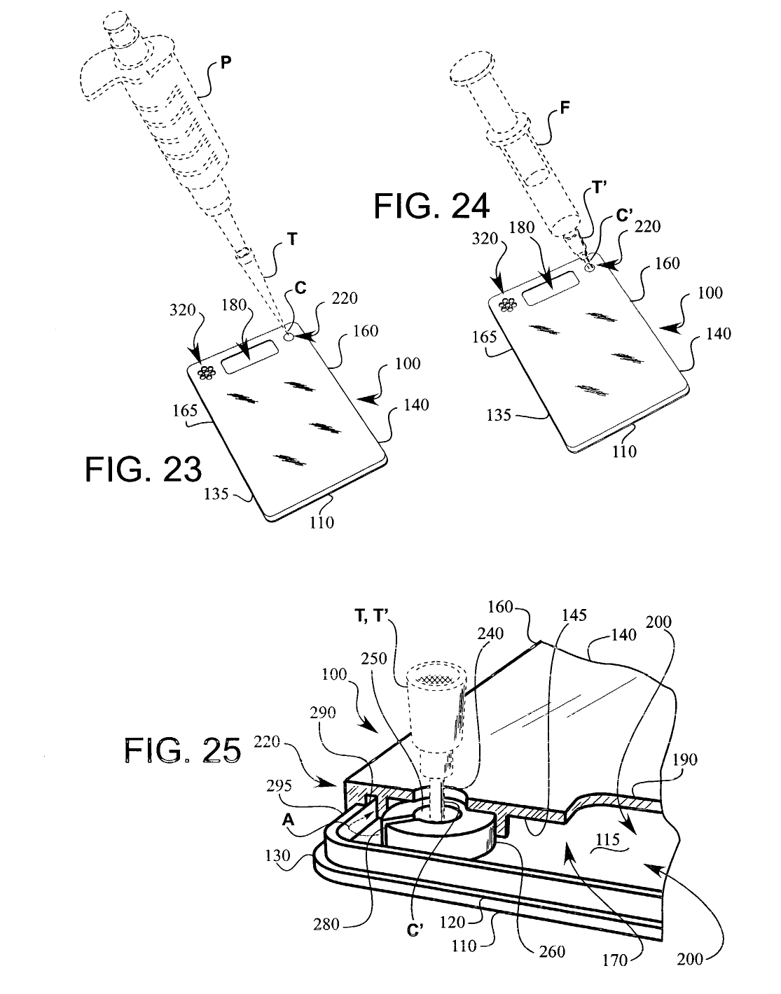

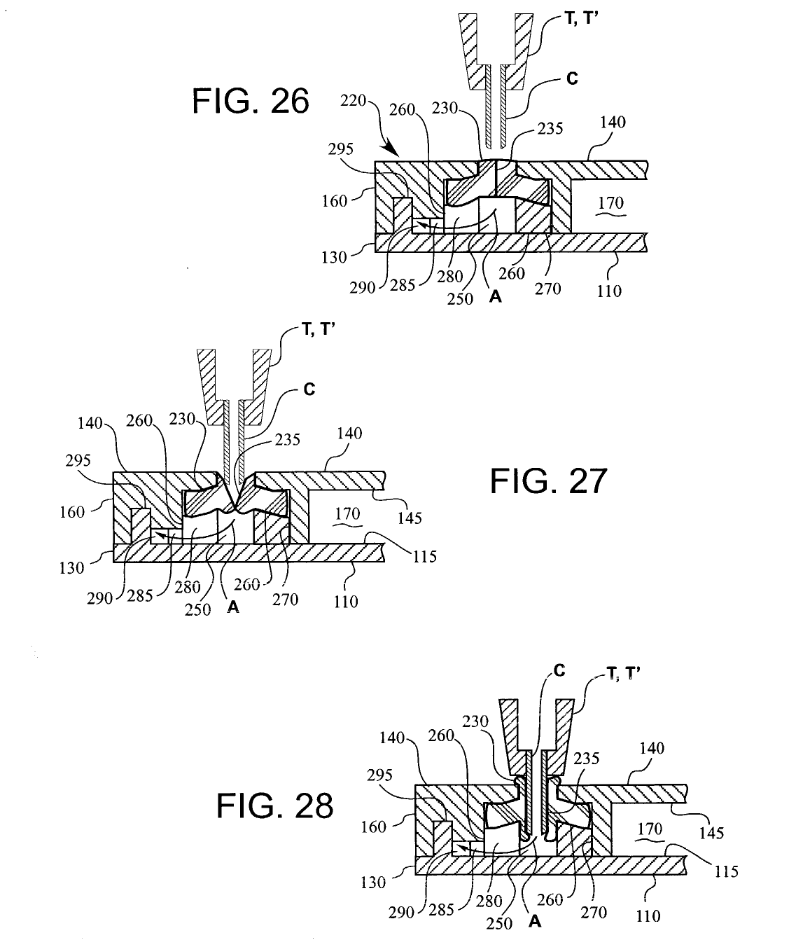

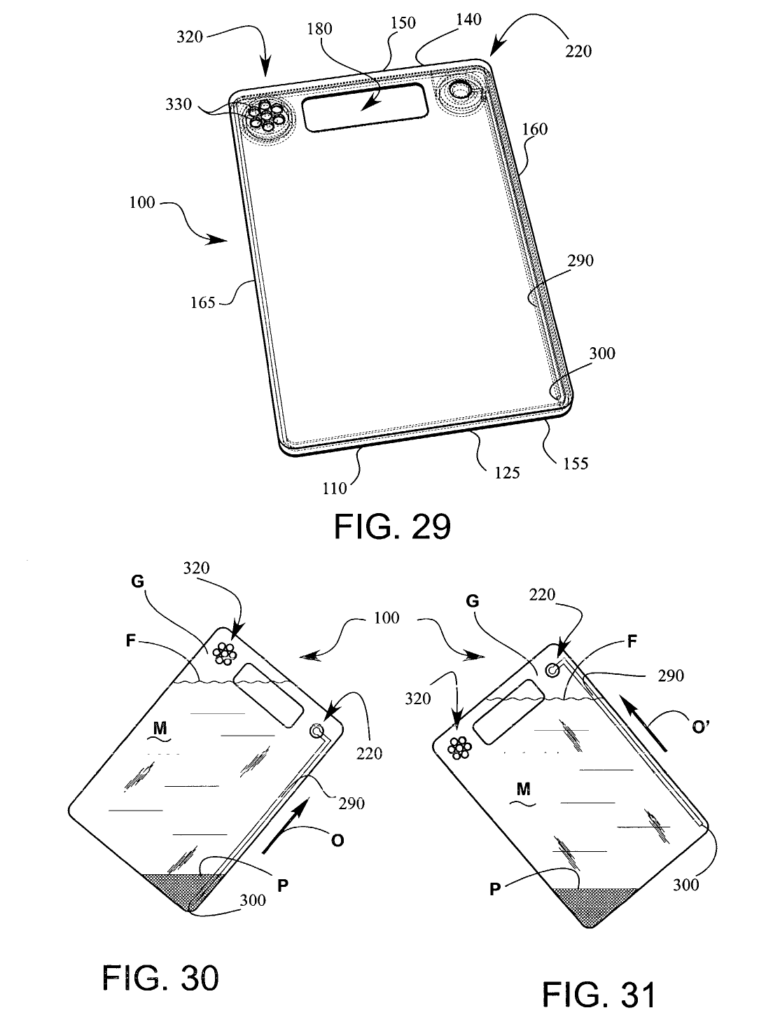

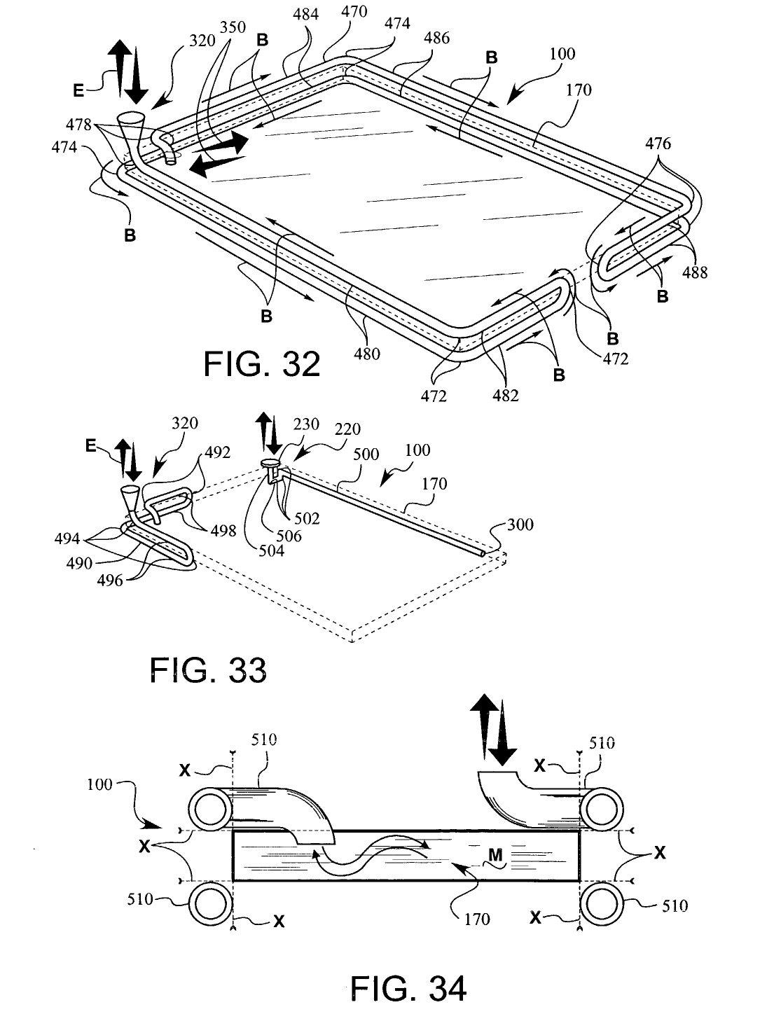

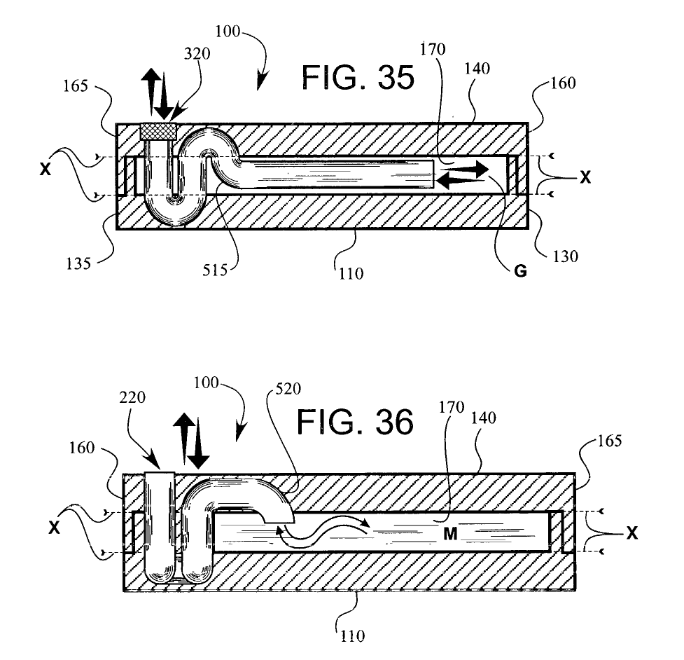

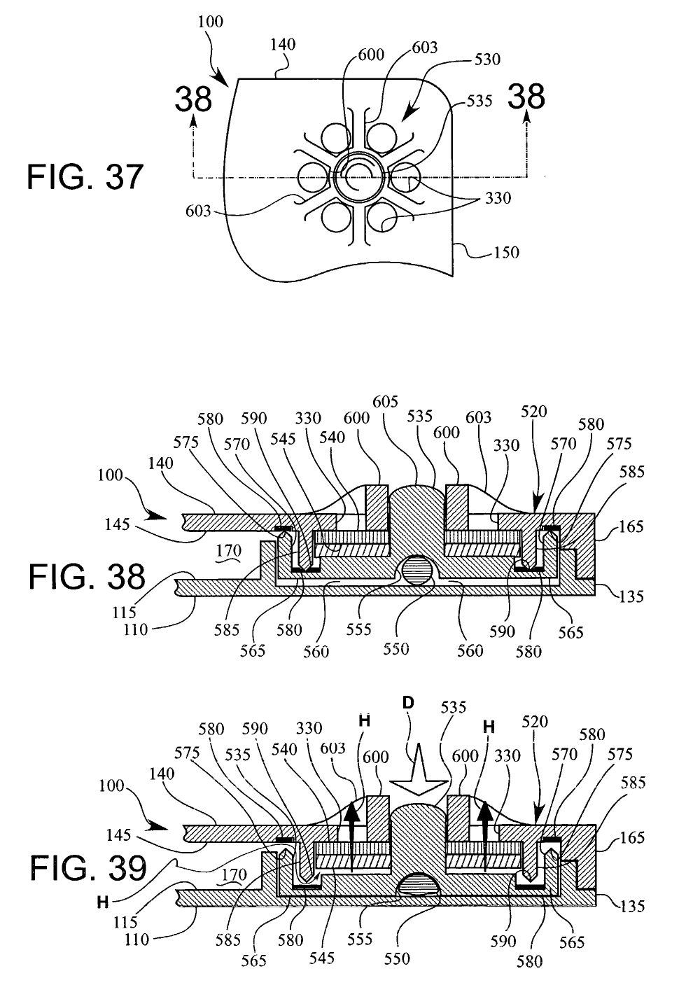

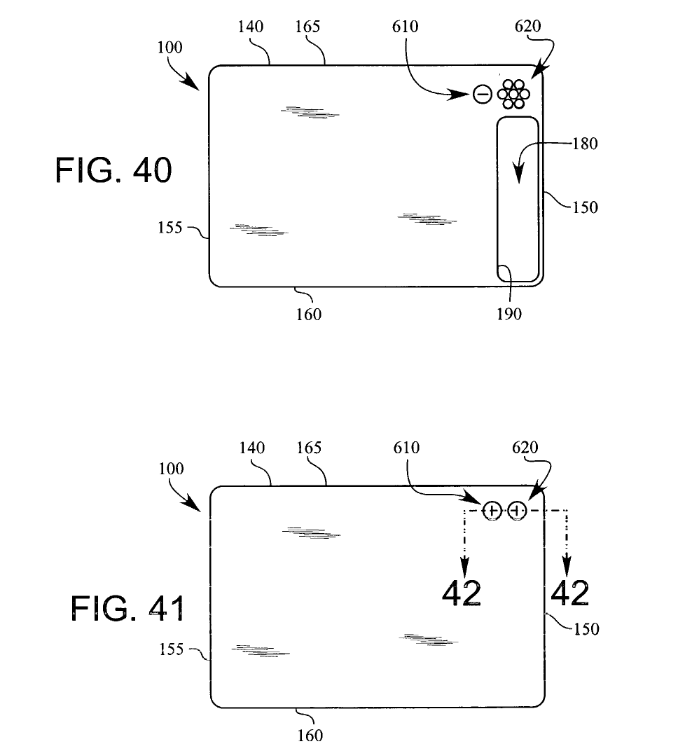

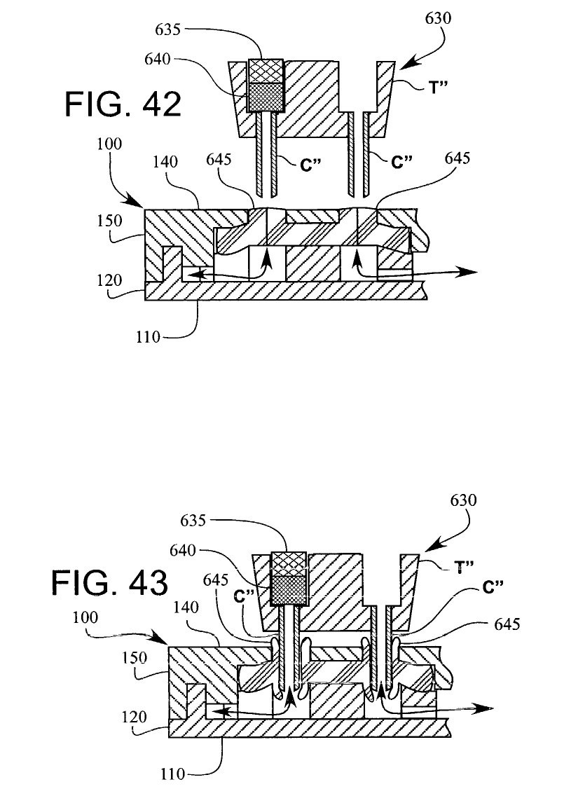

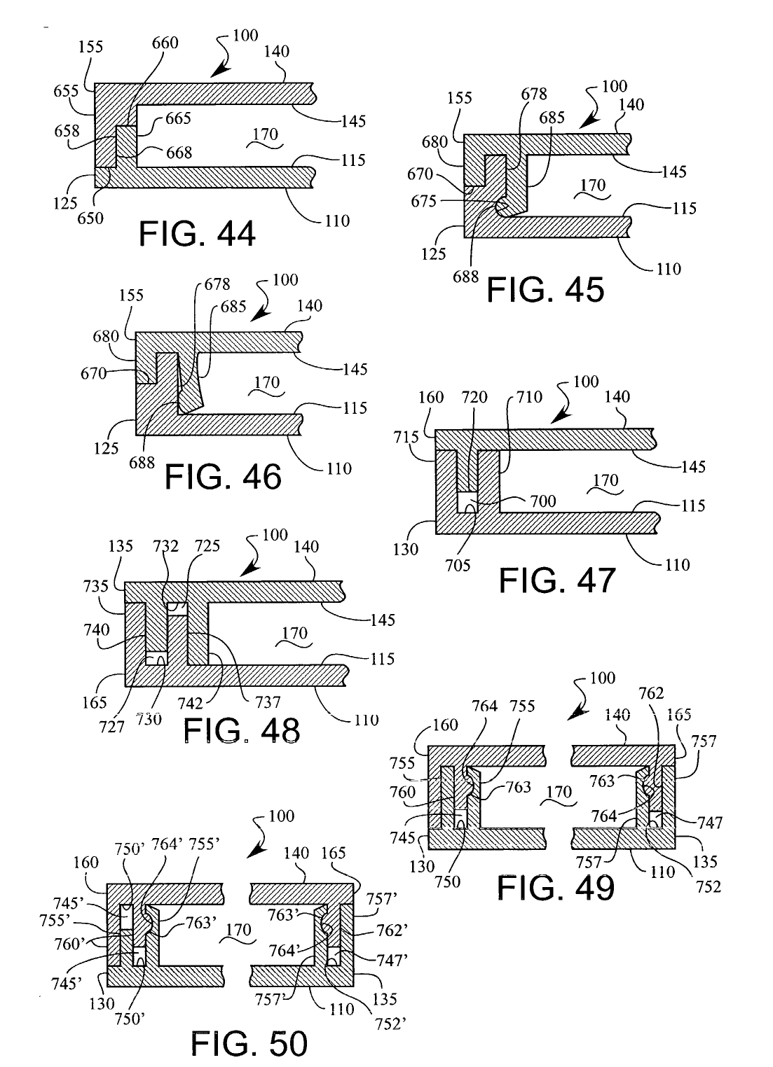

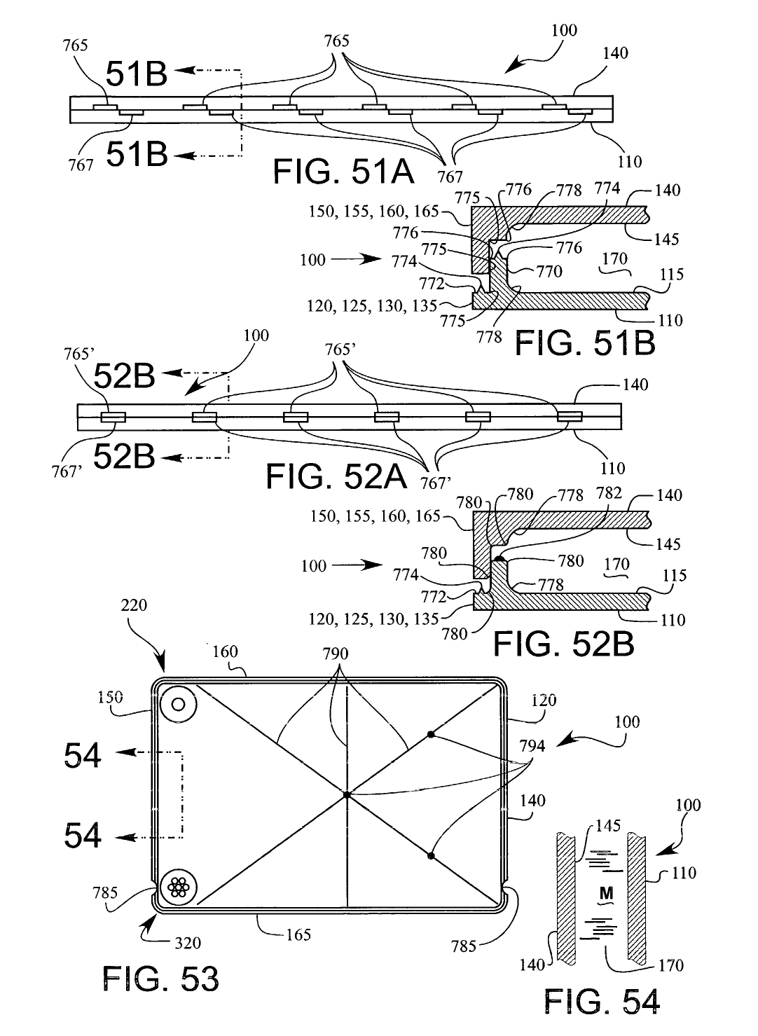

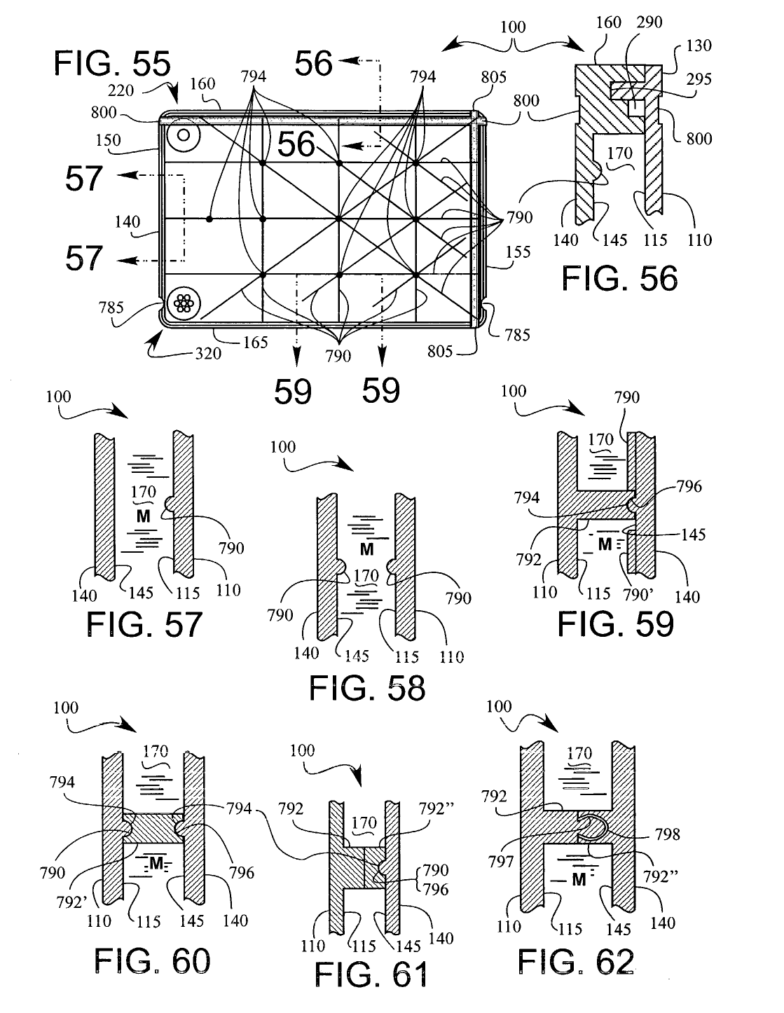

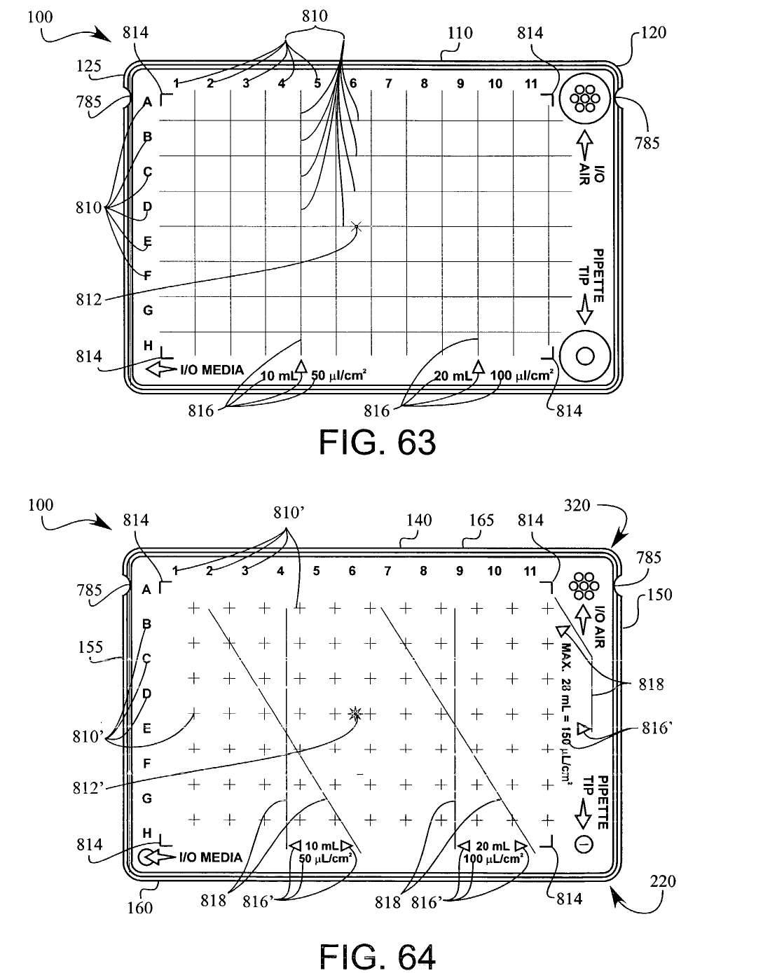

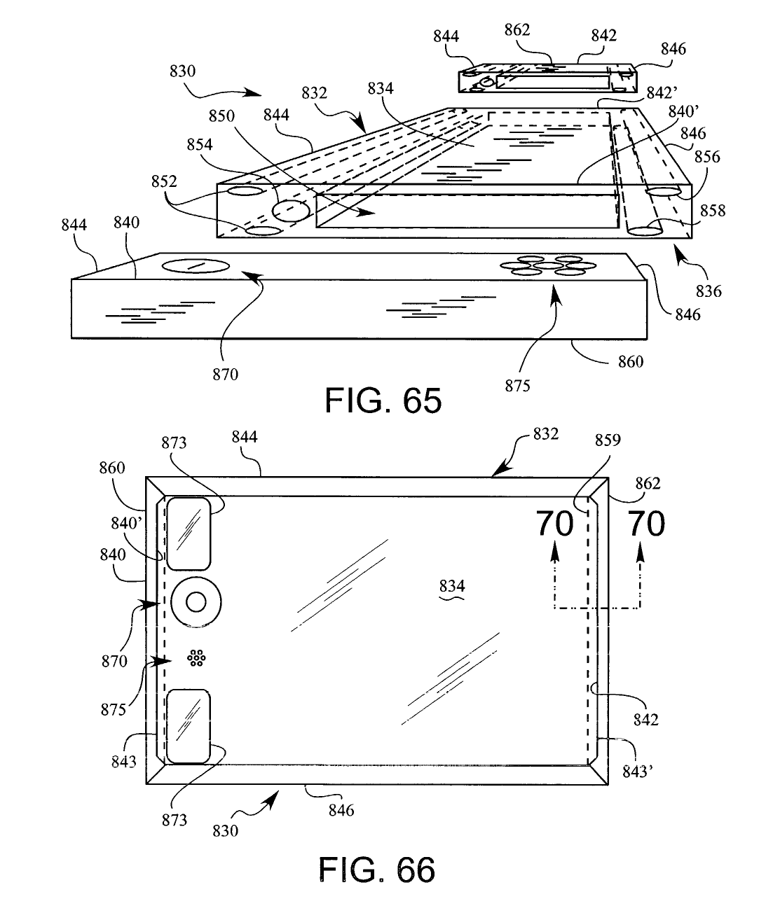

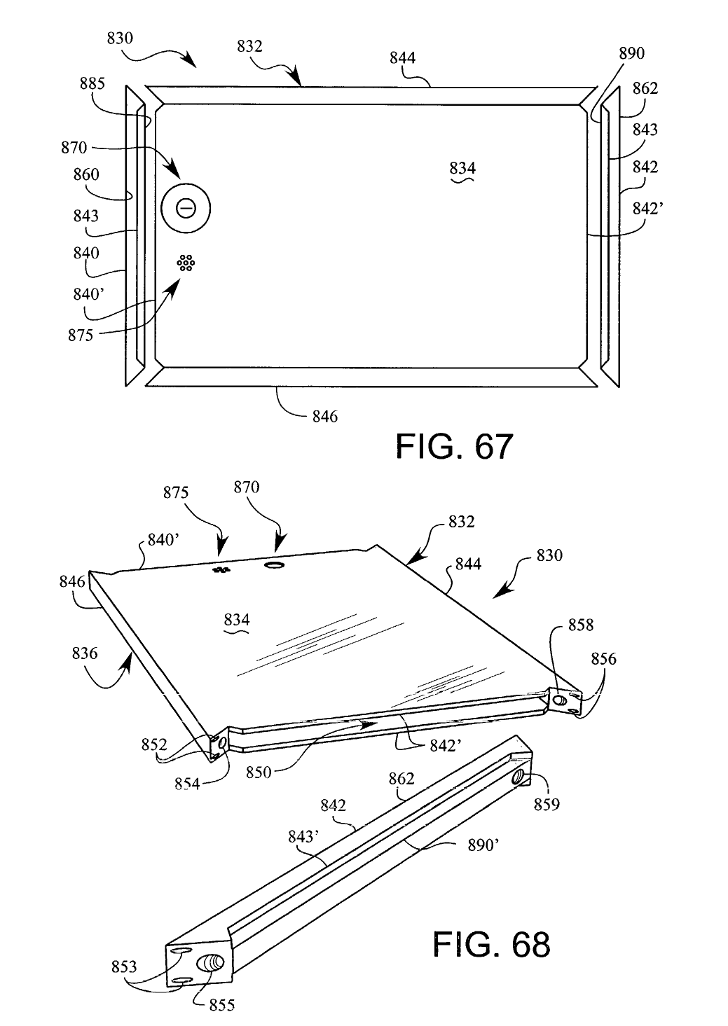

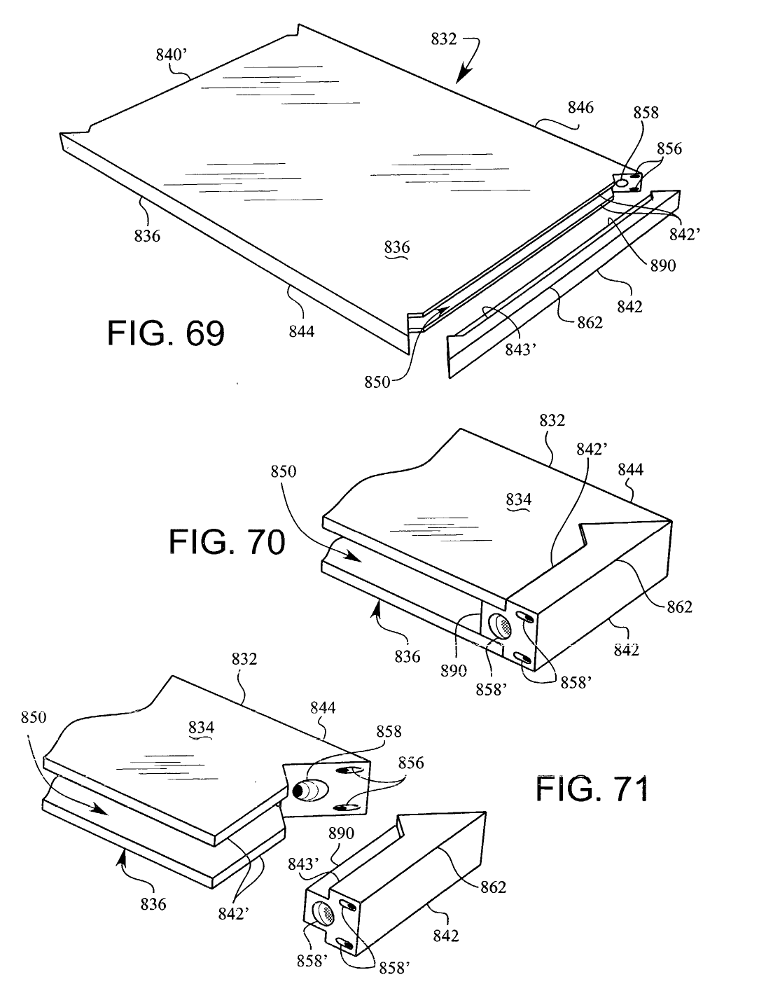

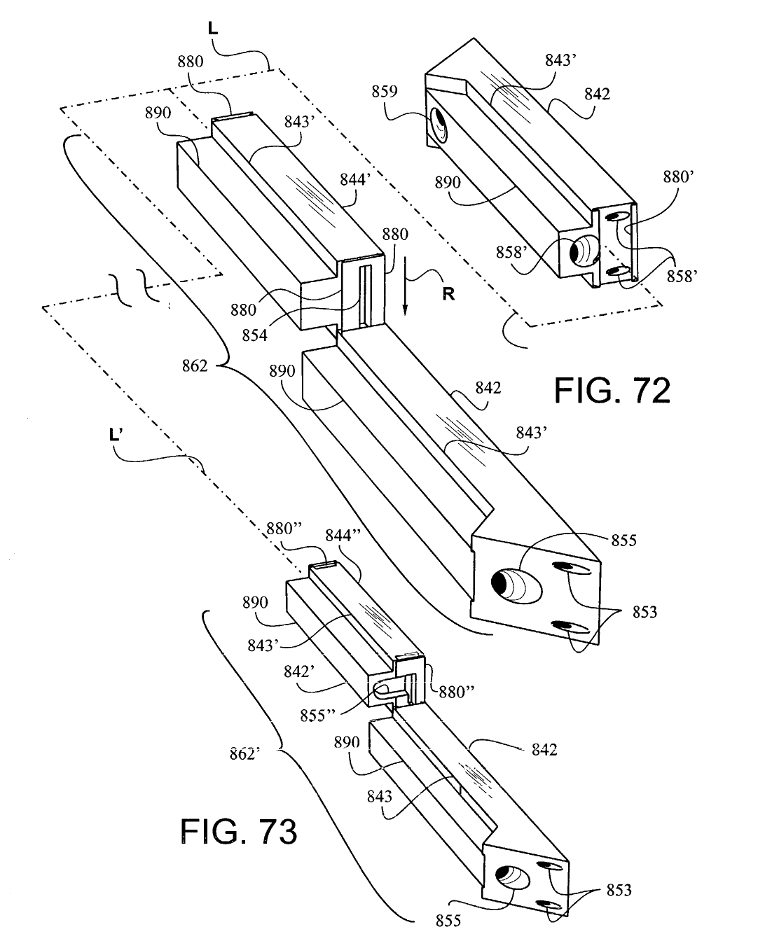

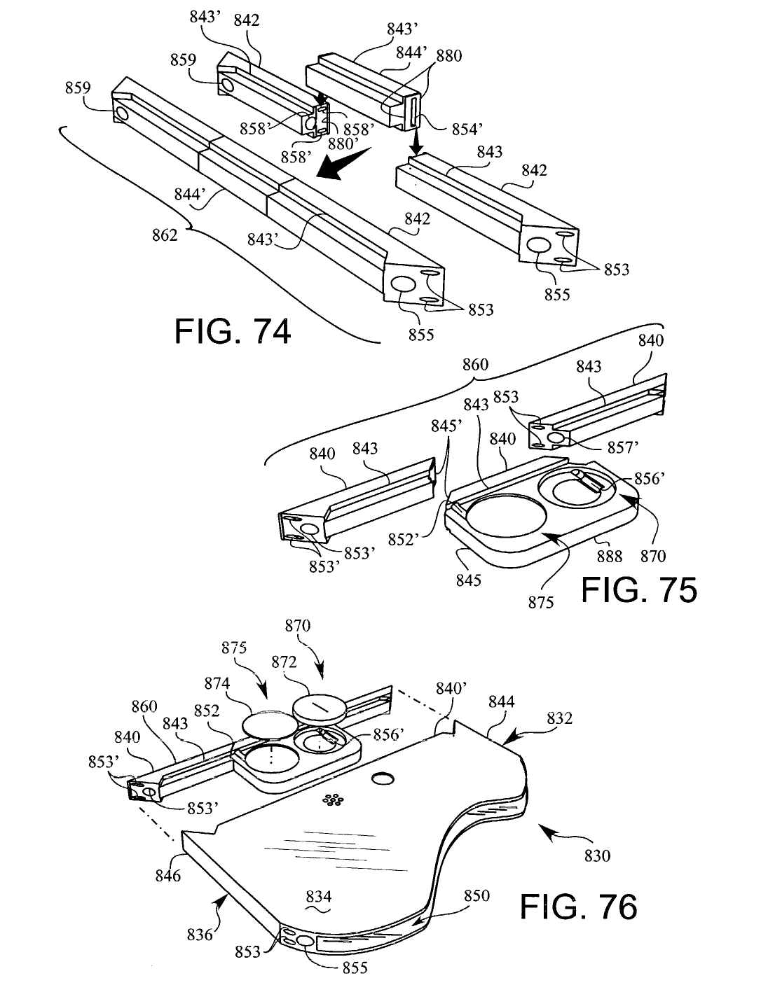

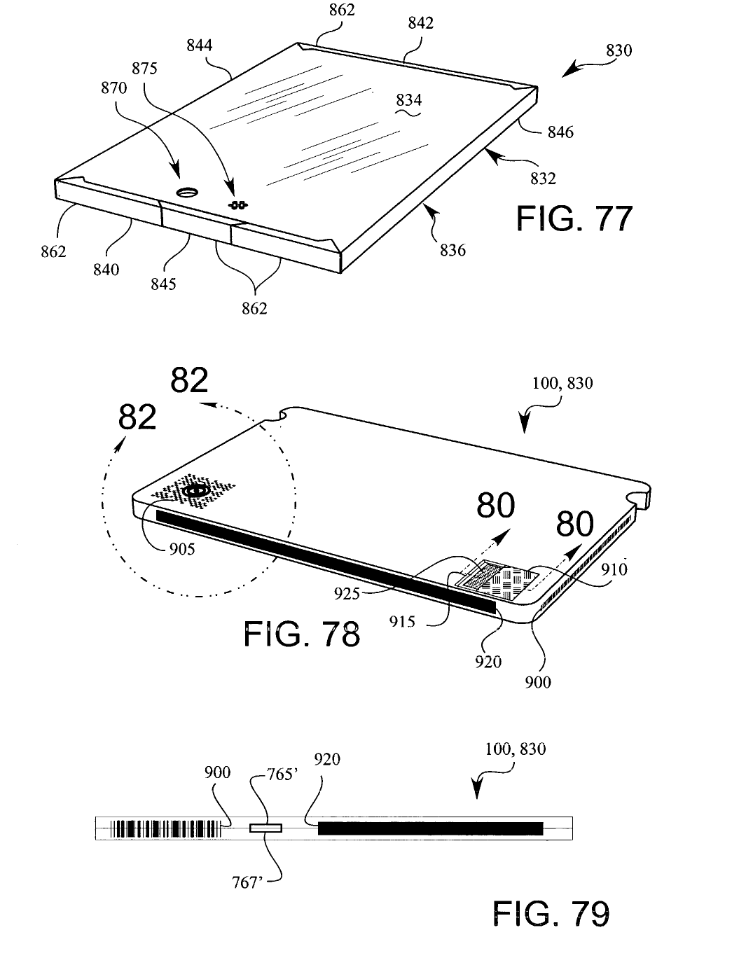

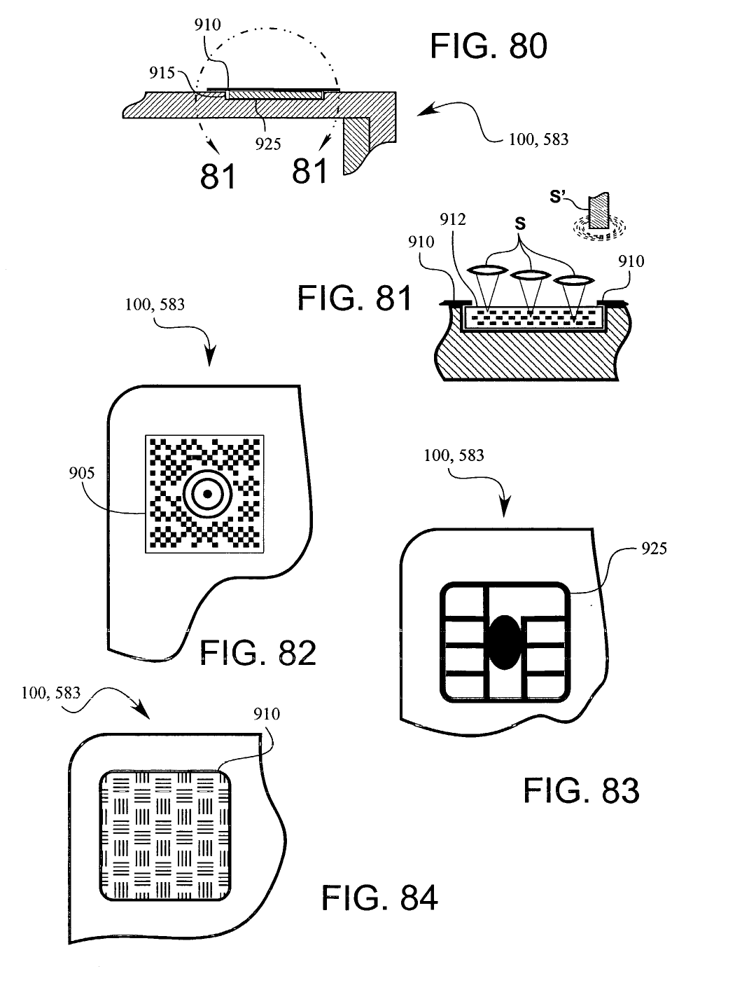

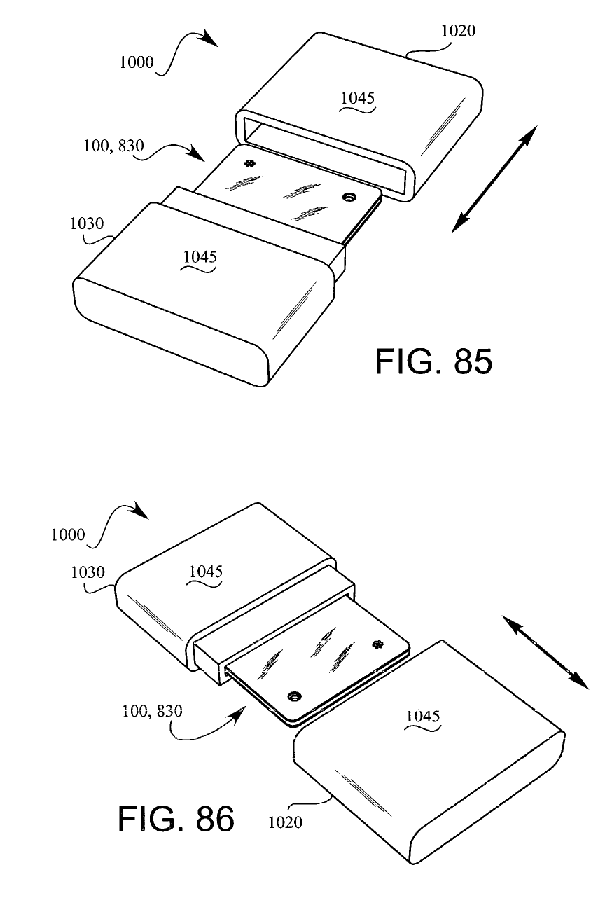

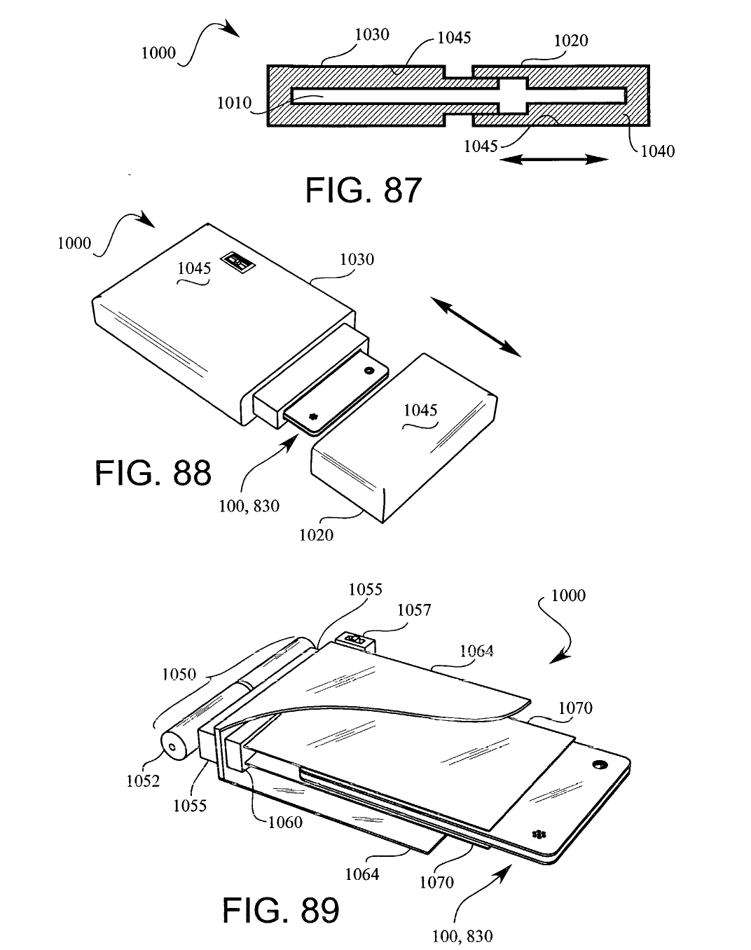

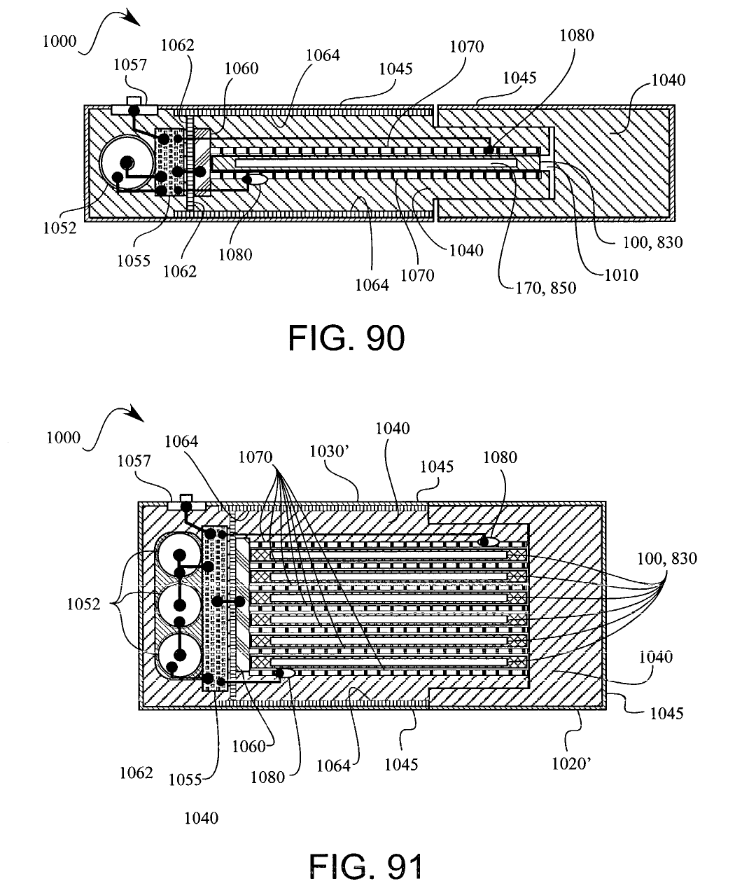

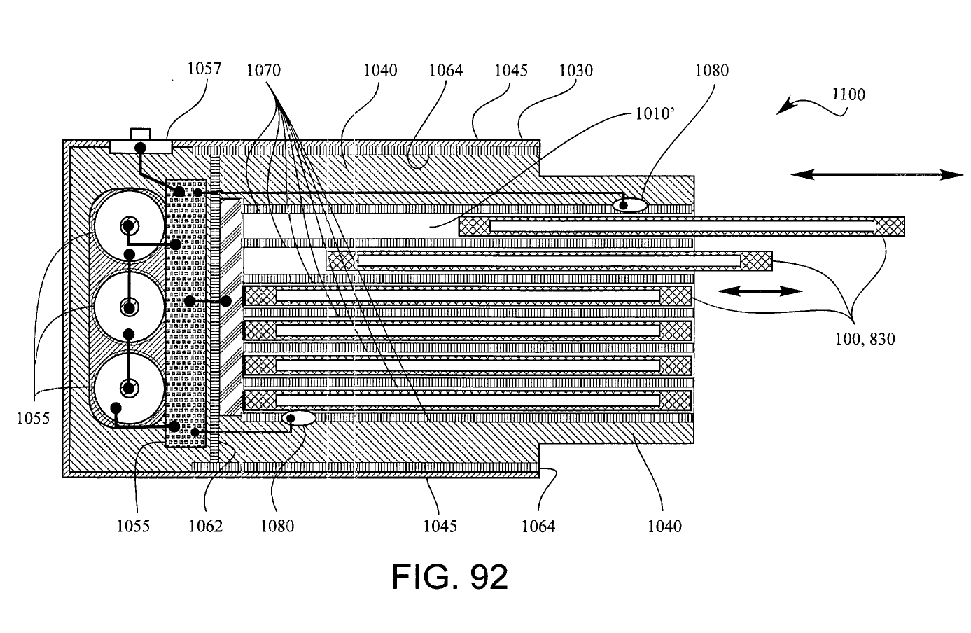

FIG. 18 is a plan detail view, rotated and in enlarged scale, of selected elements of FIG. 14 and with various structure removed for purposes of explanation;

FIG. 19 is a plan view, rotated and in reduced scale, of the device of FIG. 1;

FIG. 20 is a section view, rotated and in enlarged scale and taken along section line 20-20, of the cell culture flask of FIG. 19;

FIG. 21 is a detail section view, rotated and in enlarged scale and taken along section line 21-21, of the device of FIG. 19;

FIG. 22 is a detailed section view, in enlarged scale and taken about detail view lines 22-22, of the device if FIG. 21;

FIGS. 23 and 24 are perspective views, rotated and in reduced scale, of the device of FIG. 1 and shown in operation;

FIG. 25 is a detail partial-section and perspective view, rotated and in enlarged scale, of various components of the cell culture flask of FIGS. 23 and 24 during use;

FIG. 26 is a partial detail section view, in enlarged scale and taken about detail view line 26-26, of the cell culture flask of FIG. 21 in operation;

FIG. 27 is a partial detail section view of the flask of FIG. 26 during continued use;

FIG. 28 is a partial detail section view of the flask of FIGS. 26 and 27 during continued operation;

FIG. 29 is an elevated perspective view, in reduced scale, of the device of FIG. 1 with hidden lines depicted for purposes of illustration;

FIGS. 30 and 31 are perspective and rotated views, in reduced scale, of the flask of FIG. 1 shown in use;

FIGS. 32 and 33 are diagrammatic and schematic representations, in modified scale, of alternatives and variations of certain elements and components of the cell culture and tissue flask or vessel of FIGS. 1 and 29;

FIGS. 34, 35, and 36 are diagrammatic and schematic cross-sectional representations, in modified scale, of various additional variations, features, and elements of the cell culture and tissue flask or vessel of FIGS. 1 and 29;

FIG. 37 is a partial detail view, in enlarged scale and rotated and taken about detail view line 37-37, of an optionally modified configuration of the various features and elements of the cell culture and tissue flask or vessel of FIG. 19;

FIG. 38 is a partial detail section view, in enlarged scale and rotated and taken about detail view line 38-38, of the flask or device of FIG. 37;

FIG. 39 is a partial detail view of the flask or device of FIG. 38 shown in operation;

FIG. 40 is a plan view, in reduced scale and rotated, of an alternative arrangement of the cell and tissue culture device according to the instant invention;

FIG. 41 is a plan view, in reduced scale and rotated, of another variation of the vessel or flask according to the principles of the instant invention;

FIG. 42 is a partial section view, in enlarged scale and rotated and taken about section line 42-42 of either of the cell and tissue culture vessels or flasks of FIGS. 40 and 41;

FIG. 43 is a partial section view of the flask or vessel of FIG. 42 shown in operation;

FIG. 44 is a partial section detail view, in enlarged scale and rotated and taken about section line 44-44, of the cell culture flask of FIG. 19;

FIGS. 45 and 46 are partial section detail views of alternative arrangements of features of the flask of FIG. 44;

FIGS. 47, 48, 49, and 50 are partial section detail views, rotated and in enlarged scale, of various other arrangements of the features of the flask of instant invention of FIG. 1 and the other figures herein;

FIG. 51A is a side view, in modified scale and rotated, illustrating optional features and elements of the flask or vessel of FIGS. 1, 19, and the other figures herein;

FIG. 51B is a partial section view, in enlarged scale and rotated and taken about section line 51B-51B, of the flask or vessel of FIG. 5A;

FIG. 52A is a side view, in modified scale and rotated, that depicts additional possible features and elements of the flask or vessel of FIGS. 1, 19, and the other figures herein;

FIG. 52B is a partial section view, in enlarged scale and rotated and taken about section line 52B-52B, of the flask or vessel of FIG. 52A;

FIG. 53 is a plan view, not to scale, of an alternative configuration of the cell culture flask of FIG. 1;

FIG. 54 is a detail section view, in enlarged scale and rotated and taken about section line 54-54, of the cell culture flask of FIG. 53;

FIG. 55 is a plan view, not to scale, of an alternative configuration of the cell culture flask of FIG. 1;

FIG. 56 is a cross section view, rotated and in enlarged scale and taken about section line 56-56, of the flask of FIG. 55;

FIG. 57 is a section view, rotated and in enlarged scale and taken about section line 57-57, of the cell culture flask of FIG. 55;

FIG. 58 is a section view of an alternative arrangement of features of the flask of FIG. 57;

FIG. 59 is a section view, rotated and in enlarged scale and taken about section line 59-59, of the flask of FIG. 55;

FIGS. 60, 61, and 62 are section views having optional and alternative configurations of the features and elements of the flask of FIG. 59;

FIG. 63 is a plan view, not to scale, of optional features and elements of the cell culture flask of FIGS. 1, 19, and the other figures herein;

FIG. 64 is a plan view, not to scale, of optional features and elements of the cell culture flask of FIGS. 1, 19, and the other figures herein;

FIG. 65 is an elevated perspective diagrammatic view, not to scale, of an alternative embodiment of a cell culture flask according to the principles of the instant invention;

FIG. 66 is a plan view, in modified scale, of another alternative configuration of the cell culture flask of FIG. 65 according to the principles of the instant invention;

FIG. 67 is a partially exploded plan view, in similar scale, of the flask of FIG. 66;

FIG. 68 is a partially exploded view, in modified scale and with various elements rotated for illustration purposes, of the flask of FIGS. 66 and 67;

FIG. 69 is another partially exploded view, in similar scale and with various elements rotated, of the cell culture flask of FIGS. 65, 66, 67, and 68;

FIG. 70 is a partial section view, rotated and in enlarged scale and taken about section line 70-70, of the flask of FIG. 66;

FIG. 71 is an exploded detail section view of the flask of FIG. 70;

FIG. 72 is an elevated perspective view, in enlarged scale and rotated, of certain optionally modified elements of the flask of FIG. 68;

FIG. 73 is an elevated perspective view, in reduced scale, of various optionally configured components of the elements of FIG. 72;

FIG. 74 is an elevated perspective and assembly view, in reduced scale and rotated, of some of the components of FIGS. 72 & 73;

FIG. 75 is an exploded and elevated perspective view, rotated and in enlarged scale, of certain components and elements of the cell culture flask of FIGS. 65, 66, and 67;

FIG. 76 is a partially assembled and partially exploded view, in similar scale, of various components of the vessel or flask of FIGS. 65, 66, 67, and 75;

FIG. 77 is an elevated perspective assembled view, in modified scale, of the flask of FIGS. 65, 66, 67, and 75;

FIG. 78 is an elevated perspective view, rotated and in modified scale, of various optional features and elements compatible for use with the cell culture flask or vessel according to the principles of the instant invention and as reflected in any of the various figures including, for purposes of example without limitation, FIGS. 1, 19, 65, 66, and the other figures herein;

FIG. 79 is a side view, rotated and in enlarged scale, of another alternative configuration of the flask of FIG. 78;

FIG. 80 is a partial section view, rotated and in enlarged scale and taken approximately about section line 80-80, of optionally modified features of the flask of FIG. 78;

FIG. 81 is a partial section view, in modified scale, reflecting a diagrammatic illustration of optional configurations of the features and components of the flask of FIG. 80;

FIGS. 82, 83, and 84 are additional optional arrangements of features and elements of the flask of FIG. 80;

FIGS. 85 and 86 are elevated perspective views, rotated and in modified scale, of optional additional features and components of the cell culture flask of FIGS. 1, 19, 65, 66, and other figures herein;

FIG. 87 is a side section view, in enlarged scale and rotated and with certain structure removed for illustration purposes, of certain components of the flask of FIGS. 85 and 86;

FIG. 88 is an elevated perspective view, not to scale, of an alternative configuration of the flask of FIGS. 85, 86, and 87;

FIG. 89 is a schematic and diagrammatic perspective view, in enlarged scale, of various components of the flask of FIGS. 85, 86, and 88, with certain structure removed for purposes of further illustration;

FIG. 90 is a section view, rotated and in enlarged scale, of alternative arrangements of the flask of FIGS. 85, 86, 87, and 88;

FIG. 91 is a section view, in similar scale, of another optionally modified configuration of the flask of FIG. 90; and

FIG. 92 is a section view in enlarged scale, of the flask of FIG. 91, with various structure repositioned for purposes of further illustration.

### DESCRIPTION OF THE PREFERRED EMBODIMENTS

The state of the art of cell culture devices is significantly advanced on several avenues by the present invention. Industrial production of high-density and certain low-density cell cultures is needed for myriad applications including, for example, medical, research, military, veterinarian, agricultural, and related endeavors. There are many difficulties in producing large scale and economically efficient high-throughput, high-volume, and high-density cell cultures for such applications and endeavors, which difficulties are especially prevalent in the high-precision production of biologically synthesized molecules needed for the preparation of antibodies, vaccines, biological reagents and response modifiers, and the like. Each of these pursuits are often plagued with microbiological and cross-cell line contamination issues, ineffective or inefficient culture devices having only limited surface area available for adherent cell growth, and problems attendant to media and gas replenishment, to name a few of the more troublesome concerns.

The instant invention addresses all of these issues in new, novel, and heretofore unknown ways that not only overcome the shortcomings of the prior art attempts, but which also address such shortcomings without significant changes to conventional procedures and practices and with reductions in cost or operational constraints. Moreover, the instant invention accomplishes such advances and improvements without abandoning the long-used conservative strategies, regulations, and protocols well-established in the scientific, research, medical, and industrial communities that have the need for such improved cell culture devices. These benefits are accomplished in ways that enable sterile and compartmentalized high-volume or high-throughput cell and tissue culture with a precision and with a confidence of success that has never before been possible.

For purposes of illustrating the present invention, the terms bioreactor, cell or tissue culture device, apparatus, cell factory, container, culture tube, cluster dish, dish, flask, ELISA plate, multi-well plate (including single, double, quadruple, 4 by 6, and 12 by 8 type multi-well or 96 well plates), micro-incubator, micro-carrier, microplate, microslide and chamber slide, microtiter plate, roller bottle, spinner flask, vessel, high-density cell culture, and plurals and combinations of such descriptive phrases, all are intended to refer generally to any item capable of being used for purposes of culturing, handling, manipulating, storing, analyzing, and otherwise establishing, supporting, harvesting, and using cells and by-products thereof in vitro or otherwise for a variety of purposes as set forth and as contemplated herein.

With reference now to the various figures and specifically FIGS. 1, 2, and 3 the instant invention is directed to a cell and tissue culture flask, device, or vessel 100 that incorporates, among many other features, a posterior face, wall, or shell 110 and an anterior face, wall, or shell 140. Although any of a variety of shapes and sizes of cell culture devices or vessels 100 is contemplated by the instant invention, the illustrative configurations of FIGS. 1, 2, and 3 depict the walls or shells 110, 140 being generally planar and rectangular in shape with circumfluent penpheral edges, which in multipart embodiments can be adapted to registered with one another in a confronting relationship, and which in all embodiments propose that the walls or shells 110, 140 permanently and or releaseably mate with one another. As discussed in more detail herein, the walls or shells 110, 140 can be preferably also adapted for remating upon release and separation either by way of new and novel mating interfaces adapted for use alone and or in connection with a specialized release tool, which tool is not shown herein but which can be understood in principle by those skilled in the, relevant arts.

The posterior shell or wall 110 further includes an interior surface 115 generally circumscribed by a superior peripheral lateral edge 120 that is longitudinally opposed to an inferior peripheral lateral edge 125 and respective and laterally opposed dextral and sinistral peripheral longitudinal edges 130, 135. The anterior wall or shell 140 is preferably also formed with peripheral edges adapted to sealingly mate with the peripheral edges 120, 125, 130, 135 of the posterior shell or wall 110. More specifically, the anterior wall or shell 140 includes an interior surface 145 generally encircled by a superior peripheral lateral edge 150 longitudinally opposite an inferior peripheral lateral edge 155, and respective laterally opposed dextral and sinistral peripheral longitudinal edges 160, 165.

When the shells or walls 110, 140 are assembled together (FIG. 1), the interior surfaces 115 and 145 define what maybe referred to herein as an interior chamber, reservoir, or cistern 170 for containing nutrient media (not shown) and the cell culture (not shown) during use and operation of the preferred cell and tissue culture flask 100. As stated, the instant cell culture vessel or device 100 is directed to a variety of preferred shaped and configurations that may be equally suitable for purposes of improved cell culture capability. Additionally, the instant configurations reflected in the figures, including specifically FIGS. 1, 2, and 3, are for purposes of illustration but not limitation depicted with the interior chamber, reservoir, or cistern 170 that in one variation preferably has a volumetric capacity to hold between about 10 and 140 milliliters of media, cell and tissue culture, and constituents thereof. Even more preferably, the present invention is directed to one or more embodiments having a preferred volumetric capacity range corresponding to one of a number of different sizes of cell culture devices with one such size being that of the cell culture flask 100 depicted in the FIGS. 1, 2, 3. The device or vessel 100 preferably is sized whereby the reservoir or cistern 170 has a volumetric capacity of approximately between 20 milliliters and 35 milliliters, and more preferably between about 20 and 30 milliliters, and even more preferably a volumetric capacity that can receive at least about 25 milliliters of media, cell and tissue culture, and constituents thereof.

To establish the desired volumetric capacities the cell culture devices or vessels including device 100 that are contemplated herein generally are adapted to have dimensional sizes and shape profiles that are configured for compatibility with a large variety of existing and widely used scientific, clinical, and industrial peripheral equipment. Such equipment is readily available in use with existing prior art cell culture items having selected sizes, shapes, and configurations as further set forth herein. For example, the assembled cell culture device 100 is preferably adapted to have an exterior dimension between the respective posterior and anterior, sinistral and dextral opposing peripheral longitudinal edges 130, 135, 160, 165 of between about 6 and 9 centimeters, and more preferably about 7 and 8.5 centimeters, and even more preferably approximately 8.4 centimeters across.

In the exemplary and demonstrative configuration of FIGS. 1, 2, and 3 the assembled cell culture device 100 also preferably has an external longitudinal dimension between the respective posterior and anterior, superior and inferior peripheral edges 120, 125, 150, 155 of about between 10 and 14 centimeters, and more preferably approximately between 11 and 13 centimeters, and even more preferably about 12.6 centimeters. Additionally, the preferred cell culture device 100 is arranged whereby the exterior thickness of the assembled respective anterior and posterior shells or walls 110, 140 of the device 100 is approximately between 3 and 20 millimeters and more preferably in the range of about 4 to 10 millimeters, and even more preferably about 5 millimeters.

In this configuration as well as in other contemplated and described variations, the cell culture device 100 is compatible without further modifications for use with a wide variety of commercial and research laboratory devices, peripherals, and ancillary equipment adapted for handling, processing, incubating, bench-top and incubator retaining, processing, pumping and communicating fluids and materials to and from the culture, centrifuging, imaging, transporting, storing, assaying, and analyzing of the contents of such prior art cell culture devices. More specifically, such widely used ancillary and peripheral equipment is presently configured for use with various types of cell culture devices known in the art including, for example without limination, those cell and tissue culture devices described herein. Such ancillary peripheral equipment items further specifically include standard mechanical stage specimen holders for a microscope and other imaging hardware, washing devices, automated pumping and processing apparatus, microplate readers and spectrophotometers, centrifuges, elutriations, multi-rotor and microcentrifuge inserts for centrifuges, fluorescence and traditional microscopes and videoscopes and imaging microscopes, fluorometers, single and multi-channel pipettes and pipetters, shakers and tappers, and similar, analogous, and related equipment, including control devices and computers and systems adapted to control and monitor such equipment and cell and tissue culture items. Many such prior art cell and tissue culture items and the herein-described peripheral and ancillary equipment is adapted to be compatible with, for example without limitation, 12 by 8 multiwell or ELISA plates, which typically have external dimensions of about 12.6 centimeters by 8.4 centimeters with a range of thicknesses including in some variations an external thickness of at least about 5 millimeters.

In other variations of the preferred embodiments of the instant invention, as can be understood by those skilled in the art, lesser and higher volumetric capacities can be achieved by a cell culture device according to the principles of the instant invention and having one or more of the dimensions noted herein decreased or increased. For example, for higher volumetric capacities, the dimensions can be approximately doubled whereby the modified cell culture device could easily thereby establish approximately double the volumetric capacity while also maintaining a desirable exterior dimensional size and profile. In this doubled variation of cell culture device 100, the possible volumetric capacity can be approximately doubled, while the exterior dimensional size and profile would preferably be compatible for use with holders, mailers, and other peripheral items used for storing, transporting, handling, and otherwise manipulating CD-ROM “jewel” cases including the most widely used version that are about 142 by 124 millimeters and about 10 millimeters thick, as well as the thin-profiled “jewel” cases that are only about 5 millimeters thick.

For yet additional uses that are contemplated for application by the cell culture device 100 according to the instant invention, such as in applications commonly referred to as high-throughput, high-volume, and industrial bioreactor applications, the preferred cell culture device can be formed to have what may be referred to as a “quad-sized” or even larger configuration designed for even higher volume cell production capability wherein the lateral, longitudinal, and thickness dimensions can be modified to even larger sizes than those described herein and whereby volumetric capacities of approximately 140 milliliters and above can be established.

Some of the first cell and tissue culture dishes were glass devices, since at the time glass was readily available and further because the art of polymeric compounds was in its infancy when culture techniques were first developed. However, glass often is a less preferred compound for the construction of cell culture devices for several reasons. For example, glass is more expensive than many polymeric compounds, glass is typically more brittle and more likely to break, and when glass does break is more likely to generate dangerous shards. Furthermore, glass is less amenable to many desirable manipulations of cell culture substrates such as the manipulation of optical and thermal properties, the application of coatings or films and the like. Because of all of these and other limitations of glass, cell culture devices including that contemplated by the present invention are typically constructed of any of a wide range of desirable thermoset, elastomeric, or thermoplastic polymeric materials.

Preferably, cell and tissue culture device 100 of the instant invention is fabricated from a polymer material that is known to be compatible for use with the largest possible range of contemplated applications. Also, the preferred material can be selected for use in special purpose applications and environments as may be desirable. Such materials that are preferred for purposes of the contemplated applications of the instant invention are most commonly selected from the group of materials that includes, for purposes of use with any of the preferred embodiments without limitation, glass, ceramics, metals, thermoset and elastomer monomers and polymers, and monomeric and polymeric thermoplastics including, for further purposes of illustration but not for purposes of limitation, thermoplastic materials selected from any of a variety of commercially available and suitable materials including acetal resins, delrin, fluorocarbons, polyesters, polyester elastomers, metallocenes, polyamides, nylon, polyvinyl chloride, polybutadienes, silicone resins, ABS (an acronym for “acrylonitrile, butadiene, styrene”), polycarbonate (also referred to in the plastics industry as “PC”) polypropylene, liquid crystal polymers, alloys and combinations and mixtures and composites thereof, and reinforced alloys and combinations and mixtures and composites thereof.

There are a variety of suppliers of such polymeric compounds available for the applications for which embodiments of the present invention are to be used. One such supplier is Dow of Midland, Mich., USA, one of many manufacturers of virgin and recycled polystyrene and other polymeric compounds, manufacture crystal polystyrenes including Styron 615APR, Styron 666D, Styron 675, Styron 678C, Styron 685D, Styron 685P, Styron 478, and others. These Dow supplied compounds differ from one another in their thermal, optical, and bioreactive properties and can be selected to accommodate a wide range of preferred characteristics as may be needed for particular applications. Nova Chemical of Moon Township, Pennsylvania, USA, another manufacturer of polystyrene and other polymeric materials, can supply, for further example without limitation, approximately 70 varieties of crystal polystyrene including 3601, 2500, 1200, 3510, and others that also offer many variations of optical, thermal, and bioreactive properties.

These are but a few of the varieties of the polymeric compounds, including for example crystal polystyrene, that are compatible for use in the cell and tissue culture device 100, and similar devices available from only two manufacturers. There are other varieties of polystyrene available from each of these manufacturers, there are other manufacturers of these and other polystyrenes, and there are many other equally suitable polymeric compounds available that are contemplated for use in the instant and where depend on the particular application(s). In light of these facts, one with skill in the art may realize that cell culture device 100 may be constructed from an enormous diversity of compounds, only some of which are listed here.

Without limiting the scope of the instant invention, several criteria can guide those skilled in the relevant art in the selection of an appropriate polymeric compound. One such criterion is the bioreactive and biocompatible properties of the material. It is often desirable to minimize leaching of the material into the nutrient medium, and visa versa. It is usually also desirable that the polymeric material have little or no detrimental effect on the cultured cells or in any of their downstream applications. It may be desirable to maximize or minimize, depending upon the particular application, the activation or inhibition of certain cellular responses by selecting a suitably capable polymeric material.

For those applications in which the cultured cells or their products or materials will of may be used in humans, plants, animals, or other organisms, it is further desirable that the polymeric material have little or no detrimental effect on the human, plant, animal, microbe, or the like. More preferably, the Food and Drug Administration will have designated the material as safe in Title 21 of the United States Code of Federal Regulations (CFR) Parts 170 through 199 and parts 800 through 1299.

Another criterion that may guide the selection of an appropriate polymeric material is the optical clarity and properties of that compound. Optical clarity may be important so as not to impede any imaging, microscopic, videoscopic, or photographic observation of the cells or cell or tissue culture. The absorbance spectrum of the material may also be a relevant selection criterion, as a variety of assays and application make use of visible light, ultraviolet light, fluorescent light, or other forms of electromagnetic radiation. In these and other types of applications, uncharacterized or unexpected background absorbance or emission of the substrate polymer material and may interfere with an assay and contribute to an anomalously high background signal, unless the polymeric material is to minimize such potentially interfering emissions.

Yet another selection criterion is amenability to the addition of pigments to the polymeric material. Such pigments may be added to part or all of device 100. This pigmentation may be, for example without limitation, incorporated to add indicia to volumetrically graduate and or to annotate the device 100 with a grid location identifier, visual orientation indicia, and volumetric measures to name a few or to color code various types, sizes or varieties of device 100 to assist the technician in properly selecting and managing cell culture when and if a plurality of devices 100 are used. Such indicia may also further include alphanumeric, barcode, and multi-dimensional scanner compatible indicia and the like to uniquely identify each such device 100 for control and identification purposes and as further described in more detail herein.

Certain pigments may also protect cells from potentially harmful radiation such as ultraviolet light or some wavelengths of visible light. This pigmentation may also stabilize light labile reagents or help to optimize an assay or detection system. One or more of a plurality of pigments can be incorporated or applied during manufacture by the supplier in a manner that may be familiar to those with skill in the art. More specifically, in certain preferred variations and modifications of the exemplary embodiment discussed herein, the cell and tissue culture device 100 may be formed with a substantially transparent polymer material that is pigmented to filter potentially harmful radiation more specifically, the preferably transparent device 100 may be formed from a pigmented polymer that filters undesirable radiation or photonic energy outside the range of between about 500 and 600 manometers. More preferably the pigmented polymer filer out photonic energy outside the range of between 550 and 570 manometers and even more preferably, only photonic energy of about 560 manometers is unabsorbed and or passed by the pigmented polymer. In yet other applications, the cell and tissue culture device 100 may be fabricated from opaque or translucent polymer compounds that can be adapted to filter all or certain frequencies of incident light or other radiation including for example infrared and ultraviolet photonic energy.

Another important factor in choosing an appropriate material is the suitability of that material for applications such as warm and cold storage, transportation and handling, centrifugation, repeated freeze-thaw cycles, irradiation, high temperature incubation, low-temperature and cryogenic storage, autoclave sterilization, non-laboratory and rugged field-use applications, use with high-threat and deadly virus materials, and or combinations thereof as well as other molecular biology, clinical, industrial, or research applications and environment. Those skilled in the art have come to recognize various thermoplastic alloys and compositions that include polycarbonate, ABS, among other particularly capable and well-suited polymeric and non-polymeric materials. Additionally, while certain elements such as the walls or shells 110, 140 of the flask or vessel 100 may be formed from, for example without limitation, polycarbonate or ABS or alloys thereof, various components such as lumens, fluid communication ports and valves and valvules of the preferred cell and tissue culture device 100 may be formed from other materials including polypropylenes, polyethylenes, and a range of other metal, ceramic, and polymeric materials.

Such specialized applications can also include use in space and defense applications wherein the operators and users may be donned with various environmental control gear including gloved space-suits and bio-hazard suits, which can be accommodated by adding handling and impact-load and drop-load resistance features (not shown) to the exterior of device 100 to facilitate easier handling in such gloved hand applications. One such impact and drop-load resistance device contemplated herein is a polymeric sleeve adapted to receive the device 100 which can be formed to prevent or minimize damage to the device 100 if it is dropped on a floor or otherwise subjected to impact point loads during handling and operational use. Another contemplated device for similar protective capability includes such a sleeve device that is adapted to receive the device 100 and to establish an increased exterior profile of the device 100 that enhances the ability of a gloved hand to grip the device 100 during such anticipated use. Such features and capabilities of these added devices can be incorporated into a single device and or integrally incorporated into, with, or onto the device 100 itself during fabrication. Also further discussed herein, these added devices may also incorporate features and capabilities adapted to insulate, cool, and warm the contemplated cell culture flask 100 so as to establish the capability for stand alone incubation, storage, transportation, and or combinations thereof.

It is also preferable that the material selected for use with device 100 be at least somewhat resistant to acids, bases, salts, or other potentially harsh or corrosive reagents, media, and buffers. A related criterion is the amenability of the polymeric material to roller manufacture, injection molding manufacture, or other preferred or possible methods of manufacture. Yet another related criterion is the suitability of the material for incorporating a coating or film, or plasma or other treatments as discussed herein. In addition to these and other criterion that may be somewhat specific to molecular biology or cell culture, additional factors such as price, availability, durability, resistance to scuffing or impact, and the like may also influence the appropriate choice of polymeric material.

In any of the large number of preceding embodiments, variations, and modifications of the cell and tissue culture device 100 according to the principles of the instant invention, it has been found that the shells or walls 110, 140 may be fabricated to have cross-sectional wall thicknesses that are selected based upon and that are an implicit function of the selected materials and the intended applications. Such wall thicknesses for embodiments and modifications thereof of the device 100 of the present invention may be made from many of the more desirable polystyrene, polycarbonate, and ABS materials, and combinations, compositions, and alloys thereof, and can preferably have a cross-sectional wall thickness of between about 0.4 millimeters and about 2.0 millimeters. Even more preferably, the cross-sectional wall thickness can be approximately between 0.8 millimeters and about 1.5 millimeters. And even more preferably, the wall thicknesses can range approximately between 0.9 millimeters and 1.1 millimeters, and can most preferably be about 1.0 millimeters in cross-sectional thickness.

For purposes of evaluating the efficacy of the preferred embodiments and variations of the cell and tissue culture device 100 according to the instant invention, and with the preceding many exemplary configurations in mind, those with skill in the art will appreciate that certain interior dimensions of the reservoir or cistern 170 can have added benefit for general and specific cell and tissue culture applications. In the prior art applications, the selected volume of media was dependant on various physical and application specific constraints.

For example, for purposes of establishing an environment for effective cell and tissue culture using certain prior art flasks and devices, the operator, clinician, or technician skilled in the art would typically select a volume of media that would not only support the culture for about 3 or 4 or 5 or more days, but which would also under most circumstances keep the cell culture completely immersed during operation and incubation. Generally, many students of the myriad sciences that involve cell culturing are often taught that, when using a standard media such as Dulbecco's Minimal Essential Medium (DMEM), it is imperative to use enough media to completely cover the cell growth surface. This approach is often further qualified wherein additional media is added to completely cover the cell growth surface when it may be placed on a non-level laboratory bench surface or incubator surface that may be out of level by about 3 to about 5 or more degrees about one or more axes in the plane of the surface.

In general terms, those schooled in the art of cell culture customarily have come to learn and to promulgate that, in the appropriate media and environment, the most commonly studied and used cell lines can grow as much as 100 times in number or more (relative to the seed cells inoculated into the host media) and can survive for 5 to 6 days or more without exhausting the vital nutrients and components of the media. The appropriate media that has been empirically determined to be satisfactory is often selected to be the DMEM noted above, which is used as the cell and tissue culture nutrient source in a cell and tissue culture container maintained in the appropriate environment that is preferably adapted to be at about 37 degrees Celsius (“° C.”) with an ambient relative humidity of about 90% at an standard atmospheric pressure of 29.92 inches or 760 millimeters of mercury (“mmHg”) or about 14.7 pounds per square inch, and with an atmospheric volumetric content of carbon dioxide (“CO₂”) of about 5% that is much more than that of normal sea level standard atmospheric air (the standard sea level atmospheric content of CO₂ is about 0.03% by volume).

To obtain the desired 100 times cell growth and 5 to 6 day survival time of the cells, the appropriate environment has also been empirically found to preferably to have enough surface area in the cell and tissue container, for adherent cell applications, wherein the cell culture can propagate to confluence as the target 100 times growth goal is obtained, and wherein there is about a 5 to 6 millimeter deep layer of media covering the cell growth surface. Further empirical results have established that the appropriate environment may also be adapted with each of these parameters implemented so as to establish a media depth in the cell and tissue culture device of about 4 to about 5 millimeters from top to bottom. This depth results in a volume of the media that equals about 400 to 500 microliters per square centimeter (“μl/cm²”) of cell culture surface area.

These exemplary parameters have been found to be satisfactory for many cell culture applications in that repeated experiments established the desired results, namely, that the cells of interest were maintained without crisis for the time period of 5 to 6 days and longer without the need to replenish the DMEM or other suitable media. Thus, it was therefore concluded that if the media was replenished every 4 days, then there was a 99% probability that the cells of interest would continue to propagate and live without crisis and without detrimental accumulation of waste products. In operation, a variety of prior art cell culture flasks and devices were tested using these well-known principles and it was confirmed that an adequate amount of media to achieve such desired results was as follows:

TABLE 1

| | | Empirically | |
|---|---|---|---|
| | Surface | Adequate Media | Media Volume Per |
| Flask Type | Area (cm²) | Volume (ml) | Unit Area |
| T-25 | 25 | 15 | 600 μl/cm² |
| T-75 | 75 | 35 | 467 μl/cm² |
| Tri-Flask | 500 | 250 | 500 μl/cm² |
| Dish 100 | 100 | 50 | 500 μl/cm² |

What has been known to those skilled in the art, is that such media, including the DMEM contemplated herein, is often pre-conditioned for various cell culture application with various types of growth factors and other substances, which can significantly increase the costs of the media from nominal costs per milliliter of pre-conditioned media to $100 US, $1,000 US, and more. Given such potentially exorbitant costs, it has been long needed to establish new methods and to develop new devices capable of minimizing the potential for media and culture contamination, as well as minimizing dehydration thereof so as to maximize the efficiency of the media by extending the possible life-span of the media before replenishment is needed. What has been impossible with various prior art devices is the capability to characterize and optimize the media per cell that is needed to ensure survival without crisis for set periods of time, such as 3 or 4 or 5 or 6, or more days before the media must be replenished.

In addition to high costs being a factor in attempts to improve the state of the art, cell culture efficiency concerns also play an important role in developing new and improved devices. More particularly, those skilled in the art can appreciate that in any cell and tissue culture application, one starts with a new container or flask and introduces fresh media into the container. Next, the seed cells are inoculated into the media with an initial density of perhaps about 400 cells per square centimeter. As the cells slowly begin to propagate, they condition the media with various molecules that create more favorable surroundings which will further enhance cell growth. The lower the volume of media present in the culture container or flask, the faster the cells can condition the media present in the flask and thereafter increase their rate of propagation.

A second vehicle of increasing the efficiency of the media for purposes of enhancing performance of the cell culture includes minimization of the surface are of the media that is in fluid communication with an external atmosphere. As those skilled in the art may understand, when any surface or portion of the media is exposed to the air or another medium, there is a constant exchange of free ions between the media and the atmosphere or liquid in contact with the media. That is, free hydrogen, oxygen, and carbon monoxide ions are exchanged between the media and the external medium or air. This in turn reduces the ability of the cells to condition the media, which slows down growth and reduces yield as a function of time. Thus, in addition to the cell and tissue culture device 100 of the instant invention being adapted to minimize such external contact of the media with another medium, the device is compatible for use with predetermined MSMs, which in combination creates a cell culture environment that is more efficient than anything conceived before.

As a practical matter, in light of the physical constraints and shortcomings prevalent in the many prior art cell and tissue culture devices, the ability to ascertain the minimum static media (“MSM”) that is needed to achieve the desired 100 times cell growth capability under described time, environmental, and other parameters has in the past and with use of prior art devices been an unnecessary if not impossible exercise. The cell and tissue culture device 100 according to the instant invention is especially well-suited to be used in connection with this newly defined MSM parameter, which is being characterized in a number dimensional units, including for purposes of example without limitation, μl/cm² and μl per cell, which parameter can now be easily characterized for every conceivable cell and tissue line in light of known experimental results and still to be accomplished analyses in new lines, tissues, hybridized, and yet-to-be-conceived cells and related materials. Using the preceding data of customary media quantities per square centimeter, it is reasonable to assume that about 460 to about 600 μl/cm² is more than enough media to preventive cell and tissue crisis for at least several days even under the physically rugged environments that subject the culture to non-level surfaces, shock loads, all sorts or movements, spillage, and the like. However, such seemingly large quantities of media can be wasteful and extremely expensive. What is still relatively uncharted territory is a capability that lends itself to a determination of the MSM needed and an operational environment that is compatible for use with such a MSM volume, which has been heretofore resistant to further study and refinement. And, even if it had been possible to ascertain respective MSMs per cell and tissue line, such MSM volumetric quantities would not have been practical for use in every cell culture activities using prior art devices for at least all of the reasons stated.

With more specific reference in context to the prior art issues that have previously established the amount of media that is needed to achieve desired cell culture results, in the average clinical or laboratory setting, the prior art flask or device may be subject to accelerations and movements wherein the liquid media would “slosh” to one side or the other of the flask or device exposing the media to the atmosphere, spilling the media and culture outside the flask or device, and shocking and leaving the cell culture without nutrient media. If left uncorrected, those skilled in the art can appreciate the obvious detrimental effect on the cell or tissue culture. In a similar vein of experience, those skilled in the art have experienced circumstances where the incubation or other lab or clinical environment where the cell or tissue culture was to be grown, included one or more resting or storage surfaces that were not “level” such that the media would unevenly cover the culture and or would leave a portion thereof uncovered with similar undesirable effects.

The undesirable effects namely being that the exposed culture and any needed by products are destroyed. The brute force solution to such problems have been to simply add more media than could possibly be needed for survival so as to ensure enough media volume is present to keep the culture covered under the anticipated adverse environmental forces. This can be a very expensive solution that wastes what can be very expensive media resources. This is especially pronounced for some types of specially prepared and preconditioned media, which can costs hundreds or thousands of dollars or more per unit of volume.

These and other problems, as described herein and as otherwise known to those skilled in the art, of such prior art lab and clinical settings and circumstances are overcome by the device 100 contemplated herein in a number of ways. First, risk of contamination of the enclosed cells of interest is minimized because the prior art head space or ambient air volume ordinarily present over the media volume is substantially eliminated due to the collapsed and generally slim profiles of the preferred devices 100 described and proposed herein. The minimized head space and air volume also in turn minimizes or eliminates the bubbling, frothing, foaming, and other similar sources of possible damage to the culture that are present in nearly all prior art devices adapted for high-throughput, high-density cell and tissue culture.

Also, contamination of users and operators by contact with the enclosed media and or cells can be carefully controlled and eliminated since both media and cells cannot be spilled as has been prevalent with prior art devices. By use of the cell and tissue device 100 according to the instant invention, cells and media are completely enclosed and protected from undesirable exposure while being accessible for all purposes, and with use of generally easy to implement procedures for use that are readily compatible for use with existing and long-established cell culture protocols, such cells and media can be cultured, assayed, inspected, harvested, and the like without the possibility for any direct contact with or exposure to such users and operators.

Next, excess media volume beyond that needed to supply the cell and tissue culture with needed nutrients and other substances can be eliminated. Since the cell and tissue culture device 100 of the instant invention drastically improves protections against dehydration of the media and cell and tissue culture, and since spillage and non-level incubation surfaces become moot concerns, the only volume of media needed for purposes of the instant invention is that which is optimally needed to supply nutrients to the cells and tissues of interest and, as required, to fill the reservoir or cistern 170. As a result, such media, with the advent of the novel and inventive device 100, can be precisely titrated to accommodate the general or specialized cell and tissue applications contemplated for use by the cell and tissue culture device 100. Additionally, the instant invention further captures the many benefits of roller type flasks and devices of the prior art wherein the available surface area for cell and tissue growth is maximized, without the attendant problems of unnecessary air volume space, excess and wasteful media volume requirements, and without what can be undesirably large shearing and otherwise disturbing turbulence present in the constant motion roller devices and flasks.

Continued empirical analyses of varying quantities of media per unit of surface area have been undertaken to ascertain whether media to area volumes can be further reduced from the 460 to 600 microliters per square centimeter ranges noted herein that have been used in the prior art devices. To this end, some of the more difficult to culture cell lines have been studied to ascertain the MSM needed to establish survival of the cells of interest without crisis for at least 5 to 7 days without the need to remove and replenish the media. More specifically, human bone marrow stem and stromal cells were found to be capable of propagating to confluence and surviving at least about 5 and for as long as about 7 days inside a capillary lumen having an inner diameter of 1 millimeter using a DMEM incubated under the standard parameters set forth herein. These results establish that an MSM of 32 μl/cm² was sufficient to avoid cell crisis for the noted period. See, Murphy, M. J. Jr, Fushimi F., Parchment R. E., Barbera-Guillem E., Automated imaging and quantitation of tumor cells and CFU-GM colonies in microcapillary cultures: toward therapeutic index-based drug screening. Invest New Drugs, 1996; Vol. 13(4), pp. 303-14.

Another study unrelated to the development of the instant invention ascertained that high-density cell culture could be obtained without crisis with only 0.1 nanoliters of standard media per cell being supplied or replenished to the cell culture every 10 hours (0.24 nanoliters of replenished or perfused media per cell per day) was sufficient to prevent culture crisis. Butler, M., Growth Limitations in High-Density Microcarrier Cultures, Dev. Biol. Stand., 1985, 60:269-80. Those knowledgeable in the relevant arts may be familiar with the empirically established and traditional practice that accepts that a satisfactory media perfusion replacement rate for high-density cell culture is about 10% of the media volume per hour, which maintains a viable cell and tissue culture without crisis.

In yet another example, cells were cultured on standard chamber-slides or well-plates, which cells included for example murine melanoma cells, murine mammary carcinoma cells, human prostate cancer cells, to name a few. The cells were cultured under that standard parameters noted herein and in wells having about 36 square millimeters of available cell growth surface. Varying amounts of media were supplied in the wells of between about 50 and 300 microliters (respectively, 150 to 900 μl/cm²). Each of the cell lines grew and propagated as expected for about 7 days with the same anticipated yield and behavior.

From these studies and accepted parameters, those skilled in the art may further postulate and conclude that, depending upon the cell and tissue culture of interest, and assuming an average mammalian cell line of, for example without limitation, Hela cells (the Helen Armstrong cancer cell line having cells with an average volume of about 0.002 nanoliters), that 1.2 nanoliters of a standard media such as DMEM, will be sufficient to sustain growth and propagation from 1 cell to confluence and to maintain the confluent cells without crisis for about 5 days.

Next, for purposes of establishing an exemplary MSM for the Hela cells, it is further assumed that a confluent cell monolayer may include as many as between about 10⁵ and 2×10⁵ cells per square centimeter of attachment surface of a prior art cell and tissue culture device. Thus, if a T-25 flask is used to culture the Hela cells and if 1.2 nanoliters per cell is adequate MSM, then a volume of between about 120 and 240 μl/cm² is sufficient to achieve the desired results over the preferred 5-day time span. Although a monolayer culture is contemplated here for purposes of illustration, the instant cell and tissue culture device 100 contemplated herein is also well-suited for multi-layer cell and tissue culture applications presently known to those skilled in the art as well as the many new multi-layer and 3 dimensional pseudo-tissue culture applications currently being contemplated, debated, developed, and under investigation.

For the proposed T-25 flask of the instant rhetorical example, this means that a total MSM volume of between about 3 to 6 milliliters may be enough media to obtain the desired result and to sustain the culture for the 5-day period. However, as can be seen with continued reference to Table 1, in practice 15 milliliters is preferably used to avoid the many problems attendant with use of such prior art devices. With 25 cm² of usable surface area for cell attachment and customarily holding about 15 milliliters of media, the T-25 flask as used and described herein is usually configured to have about 600 μl/cm², which is nearly enough media to sustain the proposed exemplary Hela cell culture for between approximately 12 and 25 days. Clearly, such a prior art configuration, such as a T-25 flask being adapted to receive 15 milliliters of media, is unequivocally designed to waste what can be exceptionally costly media. And, just so the T-25 can be configured to prevent cell culture crisis during normal operational use of the T-25 cell culture flask.

Accordingly, those skilled in the art can appreciate that the capabilities of the instant cell and tissue culture device 100 that renders compatibility with such high-density cell culture applications where precise MSM constraints can be established and maintained, can result in significant actual cost savings since expensive media can be more efficiently used. In the context of these various proposed applications, and with continued reference to the detailed description of the preferred embodiments, modifications, variations, and alternatives, those skilled in the art can comprehend that the device 100 is particularly well-suited for nearly an unlimited range of possible cell culture applications.

With each of these considerations in mind, it can be further understood that preferred cell and tissue culture device 100 of the instant invention is designed to, in operation, be rotated or flipped end over end or side over side periodically or continuously, or combinations thereof, and with an optimized reservoir or cistern 170 having interior surfaces 115, 145 that can all be available for culture growth. Such interior surfaces 115, 145 can be optimized for such growth by any of a number of means, including selection of various suitable materials as described herein as well as use of any of a number of possible treatments of such materials as also illustrated herein. In any of the preferred embodiments and modifications and variations thereof contemplated by the instant invention, it has been determined that the preferred operational parameters and profiles of use of the cell and tissue culture device 100 includes the desirability, in certain general and specific applications, of rotating the device 100 periodically and or continuously so as to facilitate the growth of cell and tissue culture on all available surfaces, including interior surfaces 115, 145.

Since many types of roller flask and devices are known in the art, much work has been accomplished and published that characterizes the performance and efficacy of certain rotational velocity profiles for maximizing the growth of various types of culture material. To capture the benefits of such pre-existing knowledge, the preferred device according to the instant invention, accommodates such parameters by being preferably designed to have in interior minimum dimension between the inside surfaces 115, 145 of respective walls or shells 110, 140 preferably within the ranges set forth herein that accounts for how cells travel through media under the influence of rotational velocities of such roller apparatus and under the influence of gravity and Brownian motion of the constituents of the media.

The preferred interior minimum dimension further discussed herein is preferably optimized to accommodate the fact that the average non-aggregated cell contemplated for use with the device 100 of the instant invention has an average diametrical dimension of about 25 microns (25μ) and a sedimentation velocity (“SV”) under the force of 1 gravity of about 1 centimeter per 20 minutes, or about 0.5 millimeters per minute in standard media formulations. Thus, in the optimized operation of the cell and tissue culture device 100 contemplated herein, in certain incubation applications it will be desirable to maintain a constant speed rotation of the device 100 such that the cell falling under the influence of gravity, and through the media that is contained in the reservoir or cistern 170, will orbit in the media and will not touch an interior surface 115, 145 of the walls or shells 110, 140 during rotation. In certain applications, it may be preferable for the cell to continue to orbit in the media so long as the device 100 is continually rotated. For purposes of further illustration but not for purposes of limitation, the various embodiments that have been described herein will accomplish such a result wherein a device 100 that has been configured with the interior minimum dimension of 3 millimeters between the walls or shells 110, 140 can be rotated about any longitudinal, lateral, or other asymmetrical axis of the device to have an angular velocity of about 7 revolutions per hour, or about 18 minutes per turn. In this exemplary configuration, which is only one of an unlimited number of equally suitable configurations, the device 100 has been found to maintain the cells of the culture in a constant orbit within the media contained in the cistern or reservoir 170 whereby contact with the interior surfaces 115, 145 or respective walls or shells 110, 140 is minimized or even avoided. With each of these exemplary proposed parameters and configurations in mind, those skilled in the related arts may further appreciate that the capability for multi-axis rotation and movement of the generally slim and minimized profiles of the preferred embodiments of the illustrated and contemplated cell and tissue culture device 100 according to the principles of the instant invention establishes many new possible applications that were impossible with prior art devices.

Those skilled in the art will recognize that the device 100 is suitable for other applications wherein cell adherence to the interior surfaces 115, 145 is preferred and can further appreciate in light of the embodiments, modifications, and variations disclosed herein that the various interior dimensions of the device 100 can be modified along with the contemplated rotational parameters imposed upon the device 100 so as to maximize other desirable features and capabilities of the device 100. In another example, the device 100 can simply be repeatedly and periodically rotated from anterior side to posterior side several times for purposes of achieving cell adherence to all available surfaces, whereafter such adherence takes places, the device 100 can be moved or rotated only as needed to replenish or mix media or as otherwise may needed or desirable during operation.

The various preferred embodiments, modifications, and alternatives described herein are adapted to achieve a variety of preferable MSM volumetric and surface area capabilities. For example, in the configuration wherein the cell and tissue culture device 100 is adapted to have a volumetric capacity of at least about 25 milliliters and an available surface area for cell attachment of about 195 cm², the device 100 can support an MSM of between about 10 and 1000 μl/cm². can be obtained, and more preferably between about 50 and 500 μl/cm² is possible, and even more preferably between about 150 and 300 μl/cm² is preferred, and most preferably about 150 μl/cm² is obtained. With adjustment of the various dimensions of the array of possible arrangements of the device 100, a large range of smaller, intermediate, and larger possible MSM in units of μl/cm² and MSM in units of μl/cell are possible and can be easily accomplished.

Those having knowledge and skill in the relevant arts may also be equipped with experience to appreciate that even if cell adherence is undesirable in a given application, certain types of cellular material will adhere even if proper rotational velocities of the device 100 are maintained since cells can aggregate and thus have different average dimensions, profiles, and SVs. In such circumstances, the rotational velocity of the device 100 can be accelerated or otherwise adjusted incrementally and or continuously over time to accommodate such probabilities and or anticipated cell culture behavior. Even further, the behavior of various types of such contemplated cells during incubation and growth are well-known over time such that the device 100 can he used in connection with a automated incubation and rotation or carousel system that can change the rotational velocity of the device 100 incrementally and or continuously and gradually over time so as to optimize the rotational velocity profile of the carousel that may be in use to rotate the device 100.

Such capabilities of the cell and tissue culture device 100 also support use for many of the specialized applications and purposes connected with culturing of non-adherent and adherent cell types wherein it is desirable to promote and achieve culture and growth of non-adhered 3-dimensional cellular networks and tissues, which are often referred to by those skilled in the art as pseudo-tissues. In these contemplated applications, it can be preferable to maintain the cell and tissue culture device 100 under continuous movements and or rotation so as to promote the aggregation of cells into networks that may have been pre-introduced into the media before inoculation of the cells of interest into the media, and or wherein it is desired to promote the free, non-adhered and suspended aggregation of cells into networks formed thereby during incubation. In each of these specific 3-dimensional culture applications, it is further also preferred to employ any of the removal techniques known in the art and newly proposed herein for removing such pseudo-tissues and the like without or while minimizing the damage to the cultured cells and tissues to be harvested.

The exemplary cell and tissue culture device 100 can be fabricated from any of a number the described polymer materials set forth and contemplated herein. In most applications, such selected materials are preferably treated so as to further enhance their suitability for use as contemplated by the instant invention. Here, the instant invention is directed to embodiments that are treated according to the desirability or requirements for cell adherence or non-adherence to all of the available interior surfaces 115, 145 of the cell and tissue culture device or vessel 100 according to the instant invention. For such purposes, the instant invention contemplates that the inherent hydrophilic and or hydrophobic properties of each of the components of the instant invention are treated according to the required end-result: either that the interior surfaces 115, 145 be hydrophilic or hydrophobic or some combination thereof. In certain circumstances, the instant invention can also be further modified wherein certain components, elements, and features of the contemplated cell and tissue culture device or vessel are to be hydrophobic and certain other elements are to be hydrophilic. In this way and depending upon the desired capability of the device 100, the surface area of the interior surfaces 115, 145 that is available for attachment in cell adherent applications can be maximized. Similarly, for non-adherent cell culture applications, the surfaces susceptible to cell attachment and growth can be minimized, or in the alternative, the interior surfaces selected or treated to be hydrophobic can be maximized. In this regard, those skilled in the art can comprehend that unlike other prior art devices, the cell and tissue culture device 100 can be configured wherein all interior surfaces 115, 145 of the device 100 are available for cell and tissue growth.

Those skilled in the art can comprehend that the terms hydrophobic and hydrophilic respectfully refer to the water repelling or hating, and water attracting or loving, properties of various materials and or substances. Water is itself a hydrophilic molecule In terms of the physical and chemical properties of water, the water molecule has no net electronic charge. However, the hydrogen atoms of the water molecule have a positive charge and the oxygen atom of the water molecule has a negative charge. With this in mind, knowledgeable individuals would usually refer to the molecule having atoms with this arrangement and distribution of atomic charges as a polar molecule. Other types of molecules have analogous arrangements of positively or negatively charged atoms and are thus also polar molecules. It follows then that such polar molecules are therefore hydrophilic because they interact readily with water, and typically with each other as well, by virtue of electronically favorable charge interactions between oppositely charged poles of polar molecules.

For example, sodium chloride, commonly known as table salt, is soluble in water because the positively charged sodium of the salt interacts with the negatively charged oxygen of the water, and the negatively charged chloride ion of the salt readily interacts with the positively charged hydrogen atoms of the water molecule. Acetic acid, commonly known as vinegar, is also a polar molecule that has regions of positive and negative charge on each molecule. Due to its polar nature, vinegar will mix readily with water.

In contrast to polar molecules such as salt or vinegar, vegetable oil is a hydrophobic, non-polar molecule. Vegetable oil, like most other oils, is composed of long chains of hydrocarbons that have neither a net nor a localized charge. Since they lack regions of positive or negative charge, non-polar substances like vegetable oil do not mix favorably with polar molecules such as water. It is for this reason that oil and water do not mix but rather form separate, distinct layers when combined in the same container.

In addition to hydrophobic and hydrophilic molecules, those with ordinary skill in the art have also a convention or classification that terms certain molecules as amphipathic. An amphipathic molecule contains both regions that are hydrophilic and regions that are hydrophobic. Phospholipids, a principle component of cellular membranes, are amphipathic molecules, as are many proteins. Such amphipathic molecules may interact with both polar, hydrophilic substances as well as with non-polar, hydrophobic substances. For example, the milk protein casein mixes readily with water via its hydrophilic regions will also adhere to hydrophobic materials such as plastic about its hydrophobic regions. It is the amphipathic nature of milk that contributes to the residue that milk leaves behind in plastic or polymeric glasses or cups.

The disposition of a molecule, cell, or substrate as hydrophobic, hydrophilic, or amphipathic has important consequences in the art of tissue culture and in molecular biology in general. The surface of cells is hydrophilic, a fact that is consistent with the fact that animal and plant cells are bathed in water-based liquid, be it blood, serum, sap, or the like. Unlike the hydrophilic cell surface, cell culture substrates are commonly hydrophobic materials such as for example, thermoset materials, elastomers, rubbers, and thermoplastics such as polystyrenes, polycarbonates, ABSs, and other polymeric materials. As a result, most cells will adhere to or interact with such polymers minimally or not at all. This failure to adhere well adversely impacts the adherence or attachment requirement of many cells and cell lines. Proteins, on the other hand, usually have some hydrophobic regions or portions and therefore proteins often can bind to polystyrenes and other hydrophobic substrates, a result that may or may not be desirable, depending upon the application. Furthermore, specific experimental, industrial, or clinical applications may require, preclude, or be indifferent to the binding of various types of molecules such as, for example, nucleic acids, proteins, carbohydrates, or the like. It may also be desirable to prevent, minimize, and or maximize binding molecules to the substrate depending upon the objectives of a particular application.

In order to accommodate these and other experimental, clinical, and industrial cell and tissue culture applications of polymeric substrates it is possible to selectively control the binding properties of the surface of the substrate by inducing hydrophilia through adjusting the surface energy of the substrate; that is by establishing polarized regions on the polymer chains of the substrate. One example of this technology is the treatment of ordinarily hydrophobic polystyrene, for example, to render its surface hydrophilic. A manufacturer or technician might perform this type of application to render a cell culture substrate such as interior surface 115, 145 of cell culture device 100 more amenable to cell adherence. One possible type of selective binding control of treatment is plasma treatment. Plasma is usually produced by heating, burning, or electrically charging either ambient air, special mixtures of gasses such as oxygen, ammonia, and or other mixtures and combinations thereof. Plasma contains, among other components, many energized electrons, protons, neutrons, and molecules such as ions, free radicals, and others. More specifically, plasma is obtained by processes such as corona discharge or flame treatment which are also used to generate the plasma for treating polymer surfaces.

Corona discharge involves the application of a high frequency, high voltage signal from an electrode, across an air space or gap, through the substrate via interior surfaces 115 and 145 to some dielectric material. The frequency of the voltage signal is preferably often between 9 kilohertz (KHz) and 50 KHz, the voltage may approach as much as 30 kilovolts. The electrode can be a multi-blade discharge bar with a variable surface area that can accommodate a range of energy ratings. The optimal dimension of the preferred air gap is dependent upon several factors, including for example the thickness of the substrate material to be treated, the dielectric material, the discharge electrode, and the frequency applied to the electrode.

If corona discharge is not desirable, convenient, or compatible with a particular polymeric substrate, a gas flame treatment can also be used to create plasma. Like corona discharge, this type of treatment increases the surface energy of the substrate, renders it hydrophilic, and makes it more susceptible to cell adhesion. Several factors influence the efficacy of the gas flame treatment, which factors include the composition of the gasses. Most flame treatment applications preferably use methane, propane, or some combination thereof because of the well-understood properties of these gasses and because both gasses are relatively abundant and inexpensive. An optimal composition of gasses also requires adequate amounts of oxygen to ensure complete combustion to thereby maximize available plasma. Furthermore, those with skill in this art may understand that the flame structure, geometry, positioning, and thermal rating also influence the efficacy of flame treatment-mediated plasma applications.

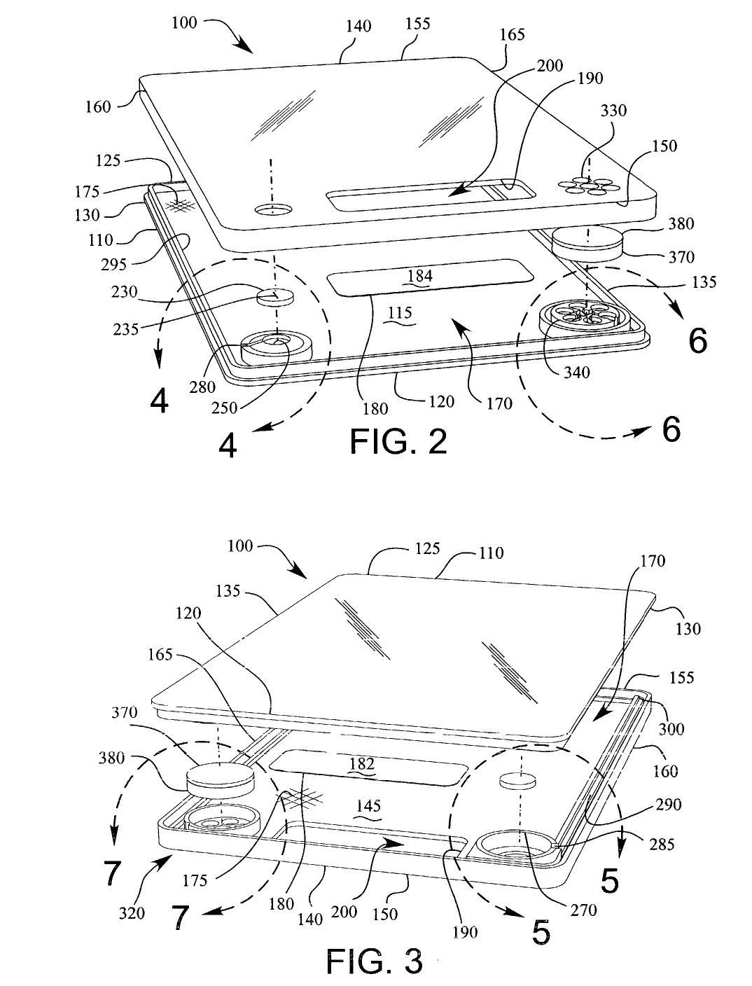

While any of these or other forms of plasma treatment can permanently modify the substrate surface to be hydrophilic, alternative treatments are also contemplated wherein an additional layer of film or a substance applied as a film is incorporated onto the substrate surface. For example, a manufacturer may apply hydrophobic or partially hydrophobic, that is amphipathic proteins to the untreated, hydrophobic surface to be treated. A schematic exemplary depiction of such an arrangement is shown generally in FIGS. 2, 3, 4, 5, 6, 7, and 20 and is denoted by reference numerals 175 to depict the treatment shown as applied to interior surfaces 115, 145 of culture device 100. Such hydrophobic or amphipathic proteins of treatment 175 may be adhered to such interior surfaces 115, 145 via hydrophobic interactions. In the application where adhered proteins are extra-cellular matrix proteins to which cells might normally adhere in vivo, this process produces a platform on the contemplated hydrophobic surface upon which cells may adhere. There are many other examples of extra-cellular matrix and structural and non-structural proteins that are or may be adapted to this type of treatment. Such proteins include, for example elastin, collagen, fibronectin, gelatin, laminin, ornathine, and the like. In addition to proteins, treatment film or layer 175 may also be or contain protein fragments, single amino acids, peptides, or polypeptides. Many of these treatments are commercially available from suppliers such as, for example, Fisher Scientific, Pittsburgh, Pa., USA.

In addition to protein treatments, a variety of forms and variations of treatments and films or layers 175 are readily available or possible. Corning Life Sciences of Acton, Mass., USA, one of many possible suppliers and manufacturers of treated polymeric compounds and substances, offers a wide range of treatments 175 that optimize or minimize the binding or proteins, carbohydrates, nucleic acids, antigens, cells, and the like. A person skilled in the art may understand that the preceding are merely examples of some of the possible treatments and coatings 175 that may be applied to polymeric compounds to render them more suited for cell culture. With any or all or a combination of such treatments or layers 175 the device 100 according to the instant invention can be optimized for general and specialized applications wherein cell or tissue culture adherence and non-adherence can be regulated as to any, all, and portions of the interior surfaces 115, 145.

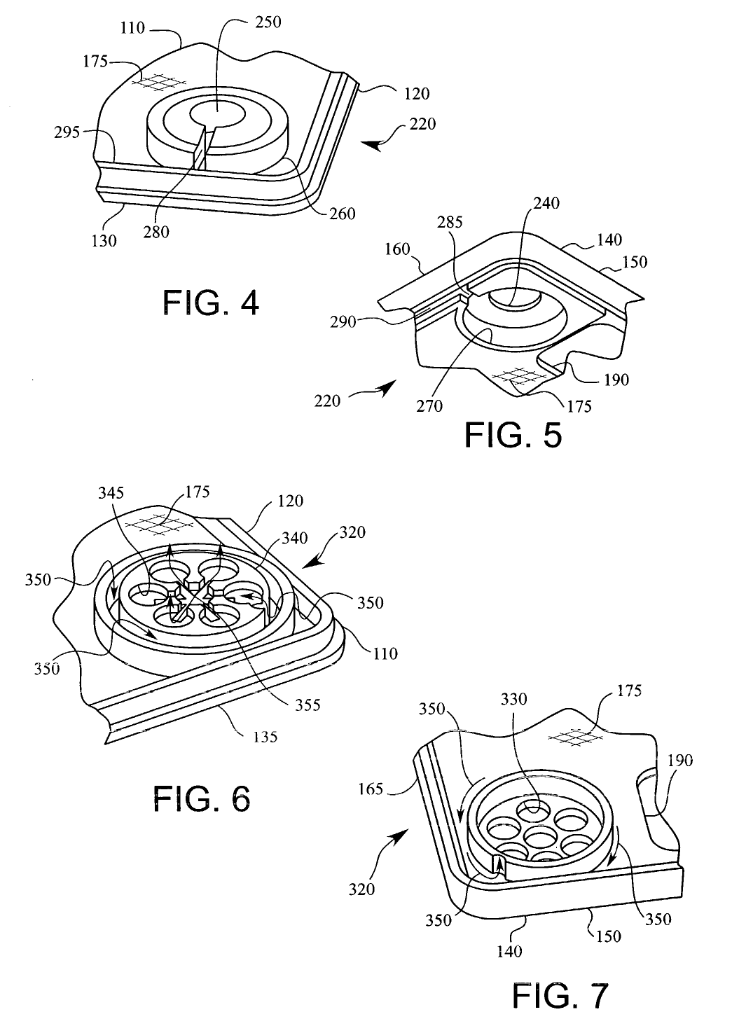

With continued reference to the various figures and specifically to FIGS. 1, 2, and 3, and now also to FIGS. 4, 5, 6, and 7 the instant cell culture device 100 also can preferably and optionally incorporate at least one respirator 180 that is sealing engaged about at least one through circumfluent interior periphery 190 defining at least one through a respirator forming aperture 200 in at least one of the posterior and anterior shells or walls 110, 140. While the various figures depict a single such membranous respirator 180, the instant invention contemplated one or more such respirators that are exemplified by respirator 180. Even though shown in the various figures and described in detail in connection with the preferred embodiments and variations, alternatives, and modifications thereof, the cell and tissue culture device, flasks, and vessels according to the principles of the instant invention have wide applicability and capability even without the incorporation of the contemplated respirator 180. The optionally included respirator may only be needed in certain applications directed to culture of eukaryotic cells and tissues having very need for gas exchange. However, even in such high gas demand applications, the materials that are selected for fabrication of the walls or shells 110, 140 of the device or vessel 100 can be selected to have gas exchange properties whereby sufficient exchange of gaseous oxygen and carbon dioxide is established with the media and cells contained in the reservoir or cistern 170 and the external atmosphere without use of the respirator 180. In other applications, including for example without limitation the culture of certain types of eukaryotic cells and certainly for most cultures of prokaryotic cells and tissues, sufficient nutrient gasses can be dissolved in the media to support the cells during incubation, which media can be regularly replenished as needed.

The optional respirator 180 contemplated herein is preferably a generally fluid impervious and gas permeable membrane, adapted, when incorporated into the instant invention, to be in physical contact with the media, in reservoir 170 on an interior side 182 and with an external atmosphere on an external atmosphere surface or external surface 184. The respirator membrane 180 is further preferably adapted to optimally communicate, transfer, and or respire oxygen and carbon dioxide, among other gases, between the exterior atmosphere and the media received in the interior reservoir of cistern 170. Even more preferably, respirator membrane 180 is selected to maximize respiration of oxygen and to minimize respiration of carbon dioxide.

In addition, the respirator membrane 180 is also preferably adapted in size, shape, location and material whereby oxygen and carbon dioxide can be communicated effectively between the external atmosphere and media within reservoir or cistern 170 with a period of time to adequately support cell and tissue growth in the cell culture device 100 of the instant invention. To accomplish such a result, the preferred respirator membrane 180 should be maximized as to surface area that is available to communicate gas.

Also, the size of the optional respirator membrane 180 should be minimized so as to limit the surface area thereof that is exposed to the external atmosphere so that the risk of rupture by accidental contract with external sharp edge items. Even more important, the respirator membrane 180 must be minimized so that loss of vapor from the media and resulting dehydration can be minimized. Even though the material of respirator membrane 180 is selected to be impervious to liquid, all liquids including the water in the media will emit vapor that can pass through the membrane 180 as gas. Further, when the size of the membrane is minimized, a larger possible range of suitable materials is available because opacity or transparency becomes less of a concern since any loss of visibility into the cell culture device is correspondingly minimized. Selection of the optimized respirator membrane 180 is also a function of several additional criteria that are dependent upon the particular cell and tissue culture device 100 that is needed in a desired application.

Such additional criteria that are generally evaluated for purposes of selecting the most optimized respirator membrane or film 180 for general and specific cell and tissue culture applications include, for purposes of example and illustration but not for limitation, (1) volume of the culture chamber or reservoir 170; (2) the differential gas permeability of walls or shells 110, 140 of the device or vessel 100; (3) the thickness of the walls or shells 110, 140 of device or vessel 100; (4) the initial gas concentration in the media, and the rate of change thereof during incubation of the media and culture, to be received in the reservoir 170; (5) the diffusion coefficient of the gases in the media received in the reservoir, cistern, or chamber 170; (6) the temperatures of the media and external atmosphere and the temperature gradient therebetween; (7) the pressure and partial pressures of the gases in the external atmosphere and the partial pressures of the gases in the media; (8) the fluid flow patterns and rates of the media received in the chamber or reservoir 170; (9) the differential gas concentration or partial pressures of the gases of the atmosphere in contact with the external side 184 of the respirator membrane 180; (10) the differential gas permeability of the respirator membrane or film 180; (11) The thickness of the respirator membrane or film 180; (12) the absolute surface area of the respirator membrane or film 180 and the ratio between that surface area and the surface area of interior surfaces 115, 145 of the cell and tissue culture flask 100.

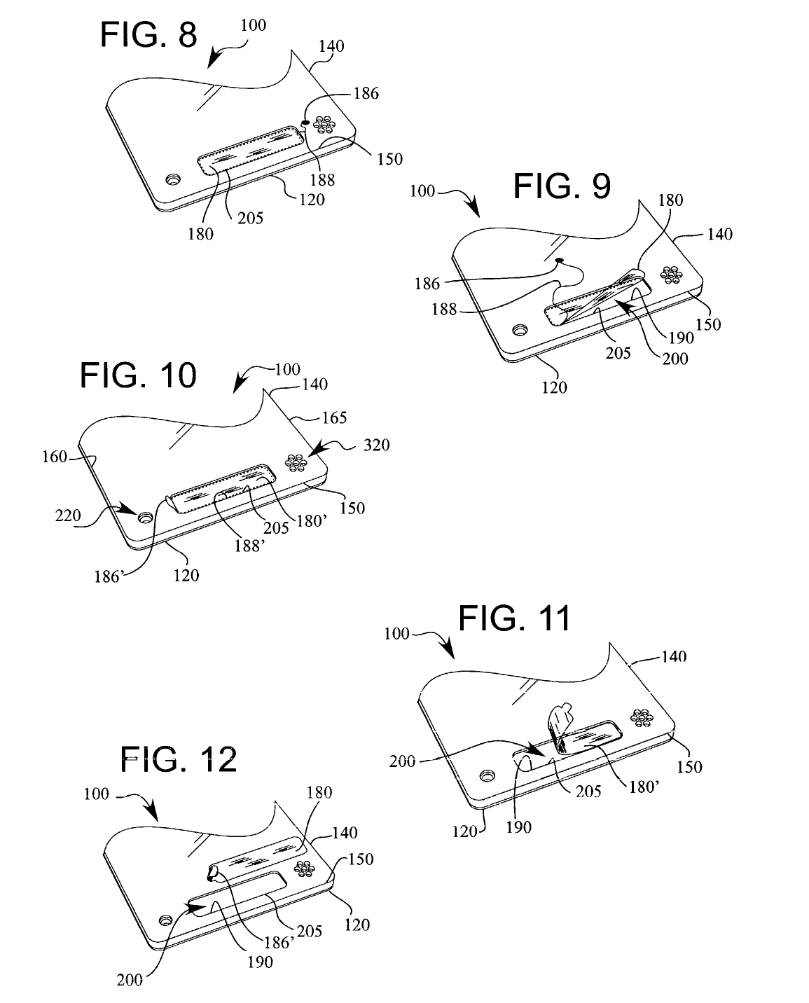

With continued reference to the various figures and now also with specific reference to FIGS. 8, 9, 10, 11, and 12, those with knowledge of the instant technology can see that in variations and modifications to any of the preferred embodiments described herein, the respirator membrane or film 180 may also be further adapted to be releasable from the cell and tissue culture flask 100 for purposes of removing cells and tissues from the device or vessel 100. In one of many possible such configurations, the membrane or film 180 is preferably hermetically sealed as already described but using a releasable adhesive or heat seal method that enables the respirator 180 to be peeled apart from the device or vessel 100.

With this optionally preferable capability in mind, those skilled in the art may appreciate from FIGS. 8 through 12 that a pull or peel tab 186, 186′ may be incorporated into the membrane or film 180, 180′ to facilitate the contemplated removal operation, wherein the tab 186 is affixed to the respirator membrane or film 180, 180′ and operates to sever the respirator 180, 180′ from the cell and tissue culture device or vessel 100. In yet another alternative, the membrane or film 180, 180′ may also incorporate a tear strand or wire 188, 188′ that is affixed to the membrane or film and that generally follows the contour of and is proximate to the periphery 190 about an outside periphery of the membrane or film 180, 180′. In the latter configuration, the tear strand or wire 188, 188′ operates to sever the outer periphery of the membrane or film 180, 180′ much like a tear off tab that forms a part of a convenient opening means of a small package of an American-style chewing gum wrapper or the similarly constructed opening means of a cellophane plastic wrapping that often protects a new, unused CD-ROM jewel case.

In FIGS. 8 and 9, the respirator 180 is shown to have tab 186 at a pull end of a rip cord 188, which in operation and as the tab 186 is pulled, the rip cord 188 is preferably incorporated onto the respirator 180 to sever the respirator 180 about periphery 190 whereby the respirator 180 is completely removed from the cell and tissue culture flask 100. In FIGS. 10, 11, and 12, the pull tab 186′ is integrally formed as part of the membranous film of respirator 180′ and can either be an extension of the film itself or another material attached or laminated thereto. Additionally, a reinforcing rip cord 188′ can be incorporated into, onto, and or as part of the film of the respirator 188′ so as to ensure that, in operation, the entire film or membrane of respirator 180′ is severed and released from the periphery 190 of the cell and tissue culture flask 100. Additionally, the respirator membrane or film 180, 180′ may also be treated with or incorporate any or all of the coating treatments described herein to treat respirator membrane 180, 180′ for purposes of achieving similar cell adherence or non-adherence results as may be desirable a particular application.

In the various alternative configurations of FIGS. 8, 9, 10, 11, and 12, those skilled in the art can further appreciate that the removal of cell and tissue culture materials and related substances from the flask or vessel 100 can be best facilitated in alternative arrangements wherein a superior edge 205 of the periphery 190 of the aperture 200 is positioned so as to be flush and or nearly flush with an interior side wall surface of superior edges 120, 150 of the respective posterior and anterior walls or shells 110, 140. In this alternative configuration, the removal of any such materials and substances can be more easily accomplished while minimizing anything left behind after removal of the majority of such materials. After the respirator has been detached as noted herein, positioning the flask or vessel 100 with the inferior edges 125, 155 directed upwards and the superior edge edges 120, 150 positioned downwards results in any media, cell materials, and related substances being poured out of now open aperture 200.

For purposes of establishing the efficacy of the preferred cell culture device or vessel 100 of the instant invention, and with the preceding illustrative embodiments, configurations, variations, and modifications in mind, one preferred version of the cell and tissue culture device 100 was adapted to be 8.4 centimeters by 12.6 centimeters by 5 millimeters thick and to have corresponding internal dimensions of 8 by 12.2 centimeters with an interior minimum dimension or thickness of the internal reservoir or chamber of approximately between 1 millimeter and 20 millimeters, and more preferably in the range of about 2 millimeters and 10 millimeters, and even more preferably between about 2 millimeters and 6 millimeters, and most preferably approximately 3 millimeters. With this configuration, the illustrative arrangement of cell and tissue culture device 100 preferably has an available interior surface area of about 195 square centimeters. For purposes of the instant invention, it has been established that such an internal surface area of about 195 square centimeters when used in connection with an internal volume of media of at least about 25 milliliters enables satisfactory results. Preferably, the media volume available for cell culture per square centimeter of surface area and per cell has been carefully titrated to have nutrient and or growth factor and other constituents with the parameters defined herein in the context of a preferred or minimum static media (“MSM”) formulations or parameter that establishes the media to be tailored to the specific application and to be sufficient to maximize cell propagation and by product yield of the culture for at least about 2 or 3 or 4 or more days without the need for replenishment. As can be understood by those skilled in the art, as the shape, configuration, volumetric capacity, available surface area, and intended cell and tissue applications of the device or vessel 100 according the instant invention are modified wherein sizes of the contemplated device 100 are selected to be smaller or larger, such quantities and parameters are similarly modified to accommodate the changes.

Many types of liquid impermeable and gas permeable materials have been evaluated to ascertain their permeability characteristics as to oxygen and carbon dioxide. The parameters that are customarily used by those skilled in the art to quantify such permeability characteristics of membranes and films are include, for example without limitation, (1) the permeability of the film or membrane in Barrers, (2) the saturation concentration of oxygen and carbon dioxide, and (3) the diffusion constants for the same gases.

Using these and other relevant parameters known to those with knowledge in the art, and using a variety of analytical and experimental methods also known to those skilled in the art, it has been determined that the example configuration of the device 100 could effectively incorporate the respirator membrane or film 180 to be a 0.125 millimeter thick Teflon EF 1600 or EF 2400 membrane material, which is available from Dow of Midland, Mich., USA. For this material and the illustratively configured device 100, it has been demonstrated analytically and experimentally that a preferred surface area of the membrane or film 180, that is optimized for (1) the exchange of oxygen and carbon dioxide sufficient to support unimpeded cell and tissue growth, and (2) the minimum possible dehydration of the media received in the reservoir 170, is preferably about 1.5 square centimeters to about 20 square centimeters. More preferably, the membrane or film 180 has a surface area available for gas exchange of between about 3 and 10 centimeters squared. Even more preferably, the membrane has a surface area of between about 4 and 5 square centimeters.

Other additional materials that may be suitable for use in fabricating the membrane of respirator 180 for purposes of the instant invention include for example purposes but not for purposes of limitation, low and high density polyethylenes, polypropylene, polymethylpentene, polyvinylchloride, polycarbonate, polystyrene, polymethylmethacrylate, polytetrafluoroethylene, and perfluoroalkoxy polytetrafluoroethylene, and DuPont's FEP product. With any individual material or any combination or alloy thereof of respirator membrane 180, it has been established that a marked abatement of evaporation and dehydration of media and cell and tissue culture is achieved over the prior art. More specifically, tests have been conducted using prior art devices and a variety of preferred embodiments and variations according to the principles of the instant invention. The test conditions included standard DMEM or similar media contained in the cistern 170 of the device 100 and contained in various prior art devices, all in an environment maintained at 37° C. and 20% relative humidity. Although many having skill in the art would recognize that cell culture is more often undertaken in an environment having a higher humidity level, perhaps as high as between about 85% and 95%, and more preferably about 90%, and lower humidity of only 20% was used to characterize the performance of the contemplated materials to be used for and the configurations contemplated for implementation as device 100. With prior art flasks and membrane containing devices it was established that about 30% or more of the media contained therein had evaporated within about 6 to 7 days. However, far less than 10% of the media evaporated of that which was contained in the similarly exposed cell and tissue culture device 100 according to the instant invention. Moreover, in variations and modifications to device 100 wherein the optional respirator 180 was omitted, no detectable amount of media dehydration occurred. In fact, using standard calculations known to those skilled in the art of thermoplastics and similar polymeric materials, it is estimated that less than 5% of the media will evaporated over a period of many months when using many of the materials contemplated herein for fabrication of the device 100.

For cell and tissue culture devices and vessels 100 that are sized differently than those described herein in connection with the illustrative embodiments, analytical and experimental results have established that the preferred ratio of surface area of the respirator membrane 180 to the internal surface area of the chamber or reservoir 170 is preferably between about 0.1% and 10%, and more preferably between about 1% and 5%, and even more preferably in the range of about 2% to 3%, and most preferably about 2.5%. For a cell and tissue culture device 100 configured as described herein, the analysis and experiments were undertaken wherein it was assumed that the device 100 was to be maintained in a motionless environment, that is, no mixing of media is induced by movement of device 100. Also, even though gas exchange will also take place through the polymeric compound or material used to fabricate the walls Of shells 110, 140, it was further assumed that all gas exchange takes place across the respirator membrane or film 180. Next, it was assumed that the media to be analyzed and used in the experiments was to be either water or a standard media such as DMEM.

With each of these assumptions in effect, and with the respirator membrane or film 180 being adapted to have a surface area in the described ranges, it has been found that, the optimum levels of oxygen and carbon dioxide in the media received in the reservoir 170 can be maintained with excess carbon dioxide being expelled and deficiencies of oxygen being replenished within only seconds. Those skilled in the art can further appreciate that the gas exchange rates will be even more optimum given that the gases of interest will also respire across the polymeric materials of the walls and shells 110, 140 of the device or vessel 100.

Accordingly, although unexpected, it has been found that contrary to custom and tradition in the art, the cell and tissue culture device 100 according to the instant invention can be completely entirely compatible for use in conventional culture applications without the need for open air exposure of the media with the ambient external environmental atmosphere. Instead, it has been found that using the exemplary hermetically sealed device or vessel 100, only between about 0.1% to about 10% of the surface area of the media used for cell growth needs to be available for gas transfer, and that that exposure can be across a liquid impermeable barrier such as respirator membrane or film 180, which will thereby minimize dehydration of the media.

Continued analysis and experiments has further established that for the cell and tissue culture device 100 having the respirator membrane 180 configured as shown herein, and for the device 100 that is configured with a volumetric capacity of at least about 25 milliliters, less then about 0.28 milliliters of water will be lost over a period of about 6 months, which dehydration minimization capability is an significant improvement over prior art devices. For cell and tissue culture devices 100 that are configured in larger sizes than those set forth here in the various illustrations, and which will presumably have respirator membranes or films 180 having increased surface areas, it has been determined that the minimization of dehydration persists.

With continued reference to FIGS. 1, 2, 3, as well as FIGS. 4, 5, 6, and 7 wherein the cell and tissue culture device or vessel 100 is shown in enlarged detail views to further incorporate at least one fluid transfer port 220 that is adapted to aspirate and receive various substances in a fluid or liquid state including, for purposes of illustration but not limitation, media, cell culture seed or inoculation cells and related materials, and by products and constituents thereof (not shown), as well as gases that may be communicated to and from the interior cistern or chamber 170 for purposes of preserving samples of air, gas or gaseous substances, and particles suspended therein.

Although much of the contextual description set forth herein is directed to cell culture and related fields, the instant invention is also contemplated for use in air and gas monitoring applications in scientific, industrial, commercial, residential, government, military environment where it may be necessary or desirable to obtain instantaneous samples of air or other gases, or to acquire such over a period of time as part of an air sample pump arrangement (which can be as simple as a battery or low voltage operated low volume diaphragm pump that is in common use with decorative home fish tanks), and for purposes of monitoring levels of various substances in such air and gas environments so that the sample can be then analyzed at another location from where the sample was obtained without further exposure to air or gas except that obtained at the sample site.

In this way, battlefield commanders can send air sample reports back to rear echelon teams that can perform detailed analyses to discover whether troops have been unknowingly exposed to otherwise undetectable low-levels or intermittent levels of chemical, biological, nuclear, and other types of enemy weapons of destruction. Similarly, government (federal, state, provincial, municipal, parish, county, etc.), commercial, industrial, and residential users can obtain instantaneous air and gas samples and samples acquired over time, which samples can then be sealed and forwarded to laboratories that can check of various contaminants, much in the same way that Americans and other nationals across the globe presently test their local water supply and their home basement levels of radon gas by sending samples to commercial testing centers. In operation, such air sampling capability would involve the use of the flask or vessel 100 in combination with air sampling equipment to pump ambient air into the cistern 170 for preservation of particulate and gaseously suspended gases, vaporized liquids, and other matter is suspended therein for later and or periodic sample analysis. As noted, this mode of operation can be useful to monitor exposure of personnel to any type of materials or substances. In various possible embodiments the flask or vessel or reservoir 100 can be put on trucks and other equipment so that the operator can maintain a record of all particulate matter to which they and their passengers have been exposed during transit.

As also noted above, this purpose can be especially useful during military operations in high-threat environments including, for example, weapons inspections in the environment of hostile dictatorships like that of present-day Iraq, which could secretly attempt to harm or injure such inspection personnel without their knowledge, or in routine anti-terror operations in Afghanistan and other parts of the world where a whole host of anticipated but prospectively unknown threats may be lying in wait for allied personnel and peacekeepers. The preferred flask or vessel or reservoir 100 can be periodically sent back to rear echelon personnel and state-side laboratories without fear of contamination during transit so that those having the appropriate forensic capabilities can ascertain what types of environments personnel may have been exposed to during such contemplated assignments.

Although many possible specific constructions of the fluid transfer port 220 are included here and in some limited aspects are known to the art, the variations and features of the instant invention are presented here in new and novel configurations. One such inventive construction of the port 220 as contemplated by the instant cell and tissue culture device 100 is more specifically depicted in the enlarged detail views of FIGS. 4 and 5. Although shown in the various figures as being accessible from a generally superior portion of a side of the anterior shell or wall 110, the instant invention is also directed to embodiments of cell and tissue culture device 100 formed with the port 220 in the laterally superior or inferior, and longitudinal peripheral edges of either or both walls or shells 110, 140.

In the illustrative construction shown in the various figures, the aspiration and injection port 220 includes a resealable elastomeric septum 230 that is preferably preslit with opening 235, which improves accessibility of the port 220 and which also minimizes the possibility that an injection aspiration access device such as a pipette tip or non-coring needle tip will core, tear, rip, or otherwise damage the septum 230 during use. A variety of equally preferably methods exist that can form an effective slit or opening 235 and one such method includes formation using a thin blade having a width of between about 1.10 and 2.50 millimeters (between about 0.045 and 0.100 inches), which width is compatible for use with a wide range of pipetter tips and other types of needles and needless lumens and cannulae that may be useful for purposes of infusing and aspirating media from the preferred embodiments of the cell and tissue culture flask of the instant invention. Also, in the configurations of the proposed fluid transfer port 220 and related elements and components shown herein, the septum 230 can be as thin as about 2 to 4 millimeters, which in contrast to many prior art attempts, substantially reduces the length of the aspiration and infusion lumen(s) of the required needle-type, needleless, and pipetter tips needed for effective port access and fluid transfer. Similar thicknesses of septum 230 are also possible in other embodiments contemplated herein but not illustrated and wherein the septum 230 is formed as part of an alternate fluid transfer port formed in any of the peripheral lateral and longitudinal edges 120, 125, 130, 135, 150, 155, 160, 165 instead of an exterior planar side wall of the shells or walls 110, 140. Although the fluid transfer port 220 is shown in the various figures as being compatible for use with various types of manually operable and automated system pipetters and fluid transfer devices, the instant invention also contemplates further modifications to the port 220 shown in detail herein that can include, for purposes of further illustration but not limitation, bayonet-type and twist-lock type compatible elements (not shown) for alternative arrangements where such positive locking and tactile feedback signaling capabilities are desirable.

The septum 230 is received within the assemblage of port 220 to be captured therein during assembly of the cell and tissue culture device 100. The assemblage of port 220 also further incorporates a recess 240 that is formed in a superior region of the anterior wall or shell 140 and substantially proximate to the respirator membrane or film 180. The recess is preferably sized to have a smaller diameter that that of the septum 230 so as to capture the septum as described. The port 220 also further incorporates a receiver and aspiration well 250 adapted and sized to receive the tip of a pipette, pipetter, needle connector or device, or needleless connector or device (see, e.g., FIGS. 23, 24, 25, 26, 27, and 28, discussed further elsewhere herein) that has, during operation, been positioned to protrude through the septum 230 and recess 240. The well 250 is defined by a septum seat 260 formed in the posterior shell or wall 110 upon which the septum 230 is captured after assembly of device or vessel 100. The septum seat 260 is registered during assembly with a port seal wall 270 that is formed in the anterior shell or wall 140. The septum seat 260 and port seal wall 270 are also formed with respective lumen ports 280 and 285 that, once device 100 is assembled, are in fluid communication with channel 290, which is sealed upon assembly by rail 295. The lumen formed by channel 295 communicates fluid between port 220 and a distal port 300 of the lumen, which port 300 flows fluid into and receives fluid from an inferior region of the reservoir or chamber 170 and proximate to the inferior peripheral edges 125, 155.

The septum 230 of device 100 functions to maintain the interior components and reservoir or chamber 170 free from contamination while at the same time giving the operator access for purposes of injecting and aspirating fluids and related substances and materials to and from the reservoir or chamber 170. The septum 230 is preferably compatible with commonly used manual, automated, and high-capacity, high-throughput liquid handling and dispensing devices and equipment such as those that may be familiar to those with skill in the art. These devices include, for purposes of example but not limitation, pipette and pipetter tips, non-coring needles, and their equivalents, and larger scale system using a plurality of such similarly configured components. The septum 230 is preferably formed from an elastomeric material such as, for example without limitation, rubber, latex, silicone, synthetic and natural isoprenes and similar materials, butyls, halogenated butyls, ethylene propylene diene monomoers, nitrites, thermoplastic elastomers, and combinations and mixtures and alloys thereof.

The choice of the most desirable material is determined by a careful consideration of intended applications, selection of desired infusion and aspiration devices, and the availability, strength, and durability of the seal to be established by the septum 230, compatibility and non-reactivity with reagents and cells and media, costs, and other similar criteria. One supplier of such materials includes, for example without limitation West Pharmaceuticals of Phoenixville and Lionville, Pa., USA, which supplies a wide range of suitable materials that can be formed into the preferred septum 230, and which can be constructed of each of the described as well as other suitable materials. Other suitable materials that have desirable properties for purposes of fabricating the septum 230 to have compatibility with the various aspects of the instant invention include natural and synthetic polyisoprenes. In the many possible configurations of such materials, many of the synthetic materials have been found to have the most desirable properties and compatibility and can have durometer ratings between about 25 and 45 on the Shore A scale, and more preferably between approximately 20 and 30, and even more preferably about 35 on the same scale. An effective material for septum 230 has also been found to have a compression set of between about 10% and 25% and more preferably between approximately 12% and 18%, and even more preferably about 16.4%. In addition to the preceding suppliers noted herein, other manufacturers produce polyisoprene materials that are suitable for purposes of the instant invention, which include 1028 gum rubber and materials having part numbers 2-6-2X 7389-35 and 2-2-3 7389-35 available from The West Company, Phoenixville, Pa., USA, and 5251 and 5218 gum rubbers available from Abbott Laboratories, Inc., Abbott Park, Ill., USA, to name a few additionally well-suited materials.

Any selected elastomer selected for use in fabricating septum 230 may be coated or laminated to further increase suitability, compatibility, or non-reactivity with reagents, media, and can be further treated with bactericides, fungicides, and other sanitization substances. One type of suitable coating materials that have been found to be useful for purposes of the instant invention and which are available also from West Pharmaceuticals, for example, include silicone based coatings as well as coatings with other inert materials such as FluroTec® or Teflon®.

With continued reference to the various figures and specifically also to FIGS. 2, 3, 6, and 7, the cell and tissue culture device 100 also incorporates a filtration and gas valvule 320 operative to maintain the optional and preferred hermetic seal between the external environmental atmosphere and the interior reservoir or chamber 170. The filtration and gas valvule 320 is also simultaneously operative to equalize the pressure therebetween during injection and aspiration of liquids and materials from port 220. The gas valvule 320 also preferably incorporates breather ports 330, filter base 340 with a fluid-gas labyrinth pathway that is formed in with the base 340 and ports 330, which labyrinth is depicted generally by arrows denoted with reference numerals 350, reflecting the fluid pathways of the labyrinth that lead into valvule 320 and into the inferior portion of base 340, and arrows 355, which lead up from the generally circular recesses 345 of the inferior portion of the base 340 and into filtration elements such as those described hereinbelow and which are preferably adapted to communication substantially if not completely sterile gas between the external atmosphere and the interior reservoir, cistern, or chamber 170.

Although the filtration and gas valvule 320 depicted in the variously illustrated figures, namely FIGS. 2 through 7, incorporating the fluid labyrinth, which directs the internal liquids and gases towards the liquid impervious filtration elements, the instant invention also contemplates the labyrinth to be in fluid communication with a siphon lock lumen (further depicted and described elsewhere herein) that is adapted to minimize head pressure of the internal media against the filtration elements during operation of the device 100. Minimization of such pressure can serve to minimize the possibility of leakage during various types of severe environmental conditions including centrifugation, transportation, and incubation under unusual and perhaps continuously changing attitudes of the cell and tissue culture flask or vessel 100. The siphon lock lumen is described further herein in various proposed alternative configurations and in certain aspects preferably can operate on the same principles of a water trap that is often incorporated into most commercial and residential plumbing drains for purposes of creating a barrier against and for trapping sewer gases and keeping such from permeating through the working and living spaces adjacent to such drains.

When the cell and tissue culture device or vessel 100 is assembled, filtration elements 370 and 380 are sandwiched between walls or shells 110, 140 such that the elements 370 and 380 are captured beneath the breather ports 330 and above the filter base 340. Although not shown in detail in the figures, those having knowledge of the relevant technology will further appreciate that any of the preceding preferred embodiments and modifications thereto can also incorporate one or more types of positive actuation pressure and vacuum relief and or check valves (including for example one-way, two-way, three-way, and other types of relief and or check valves), and combinations thereof, in place or and or as part of and in combination with the filtration and gas valvule 320. The term positive actuation is used in the context of a pressure and vacuum relief and or check valve that is actuated upon exposure to a predetermined pressure and or vacuum and which maintains a pressure and vacuum seal against unwanted communication of gas or fluids until the predetermined pressure or vacuum is established. With such an additional capability, the instant cell and tissue culture device 100 can be further adapted to enable incubation, storage, and transportation of the contents subject to such a pressure or vacuum of the predetermined magnitude.

With continued reference to the figures already noted herein and also now to FIGS. 13 and 14, those skilled in the related arts can further understand that as an added measure of contamination protection from unexpected and undesirable escape cells or media, or from undesirable contaminants being introduced into the cistern or reservoir 170 of the device 100, the filtration and gas valvule 320 is also optionally adapted to be permanently or releasably and temporarily sealed with a gas and or fluid impervious sealing device or devices 400, 440, 440′ which for purposes of illustration but not limitation can include an adhesive coated film 410, 445 or other similarly functional device that can overlay the entire opening or series of openings, such as breather ports 330, so as to seal the valvule 320 and other openings into the cistern 170 against unwanted communication of particles, fluids, and or gas.

By use of the contemplated sealing film, tape, label, cap, or device(s) 400, 440, 440′ the media and cells of interest inoculated therein can be subjected to pressure and or vacuum during incubation, storage, transportation, and operation, subject to the structural ability to withstand such forces of the materials that are selected for manufacture of the devices 400, 440 and the shells or walls 110, 140 of the device 100. Maintaining such a pressure or vacuum can be useful during transportation that may involve altitude changes that would otherwise subject the flask or vessel 100 and its contents to pressure shocks or changes that could be detrimental to such contents. By imposing a known pressure or vacuum upon the contents prior to such movement, the results can be more predictable. Moreover, certain types of cells and tissues are known to those skilled in the art to be more productive when subjected to such a pressure.

The contemplated self-adhesive film, tape, label, cap, and device 400, 440 can also be used in connection with the contemplated optional respirators 180, 180′ for many related and similar purposes. For example without limitation, such sealing film, cap, or devices 400, 440 can be shaped and sized for use to independently and or simultaneously seal the valvule 320 as well as the respirator 180, 180′ during incubation of specialized cell and tissue applications wherein it is desirable to prevent gas exchange either through the valvule 320 or the respirator 180. Moreover, such a contemplated sealing film, cap, and devices 400, 440 may be preprinted with indicia adapted to facilitate the recordation of date, time, and other relevant data that could be of import to the users, technicians, and operators during incubation, replenishment, storage, and analysis activities.

With continued reference to the figures already described and with reference now also specifically to FIGS. 15, 16, 17, and 18, it can be understood that the sealing devices 400, 440 can be configured as films or tapes 410, 410′, 440′ that be formed as multiple pieces. In one contemplated arrangement of the multiple piece configuration shown in FIG. 15, the sealing device can have a valvule seal 415, a respirator seal 420, and a fluid transfer port seal 425, each seal having respective release tabs 417, 422, and 427, and various identification and annotation indicia 430. The annotation indicia can be especially helpful locations to record dates and times of last media replenishment, or the next scheduled time therefore. Alternative arrangements are shown in FIGS. 16 and 17 wherein the reference numerals with primes and double primes correspond generally to the numerals depicted in FIGS. 13, 14, and 15.

Further to the noted capabilities sealing film, cap, and devices 400, 440 the instant invention embodied in the new and novel cell and tissue culture flask 100 and variations thereof can also further be adapted wherein the fluid transfer port 220 is adapted to be permanently or releasably and temporarily sealed with the sealing film, cap, tapes, and devices 400, 410′. As noted, the film, cap, and devices 400, 410′ may be further adapted as depicted in the various figures for independent and or simultaneous sealing of the fluid transfer port 220 along with or independent of the respirator 180 and the gas valvule 320. While such sealing film, cap, and devices 400, 410′ can be designed and adapted to prevent the communication of particles, gas, and fluids via the component to be sealed, the sealing film, cap, and devices 400, 410′ can also be further adapted to establish a protective barrier capable of protecting against inadvertent and sharp object damage to the respirator 180, the fluid transfer port 220, and or the gas valvule 320 during the various activities and environments that are contemplated for use by the device 100. One of many possible means by which to impart such protective capability includes, for purposes of explanation and illustration but not for purposes of limitation, the addition of a metallic or polymeric layer to the film embodiments of the sealing film, cap, and devices 400, 410′, which metallic or polymeric layer can be, for further example without limitation, an aluminum or steel foil, a metallicized polymeric or cellulosic material, and a high-strength Kevlar®-type woven polymeric material.

With reference also now to FIG. 18, the sealing film or tape 440′ is illustrated in a single sealing piece configuration that is sized, shaped, and adapted to simultaneously seal the fluid transfer port 220, the respirator 180, and the filtration and gas valvule 320 with a single strip of material 445 which can be imprinted similar to the imprints discussed herein above to have indicia 450 (e.g., annotation data) and 455 (e.g., barcodes and the like). Each of such indicia may also be directed to recordation of data that can include date of last cell inoculation, media removal and replacement, and the like. In any of the contemplated embodiments, configurations, variations, modifications, and alternative arrangements of the sealing film, tape, label, cap, or device(s) 400, 440, 440′, those skilled in the related technology may be able to understand that such sealing items 400, 440, 440′ can be preferably arranged and dispensed on sheets, rolls, and similar means that are often employed with analogously configured items, which are all contemplated to be compatible for use with label, laser, ink jet, and impact printers, and similar types of indicia imprinting devices whereby serialized indicia, alphanumeric data, one and multidimensional barcodes and pattern codes, and other types of optically and magnetically readable data can be printed and other types of indicia can be impressed upon the items 400, 440, 440′ according to the principles set forth herein.

The sealing film, cap, and devices 400, 410′ contemplated herein are also susceptible to incorporation of a tamper-warning capability wherein additional scoring or perforation lines (not shown but known to those having skill in the related arts and commonly employed on retail store price tags labels to prevent undetected removal by customers prior to purchase of an item) are included into the devices 400, 410′ to prevent undetected removal, and wherein additional materials and components may be incorporated and or included that are adapted to identify, reflect, and otherwise indicate a puncture and or any tampering of the sealing film, cap, and devices 400, 410′ so as to alert operators and users to unintended or otherwise undesirable interference of any sort with the sealing film, cap, and devices 400, 410′ once such have been applied to seal the components of the flask or vessel 100.

With continued reference to the various figures already described and revisiting the subject of the filtration and gas valvule 320, the primary function of the filter element(s) 370, 380 which maintain the sterility of the interior compartment, cistern, chamber, or media reservoir 170 of cell and tissue culture device 100. It is known in the art to use filter media to exclude or remove most microbes such as bacteria and fungi, larger cells, debris, and other possible contaminants from an air stream. To filter out bacteria and other microbes, for purposes of illustration but not limitation, a small pore filter such as a 0.2 micron filter or some other similarly fine porosity on the same order of magnitude is contemplated for use in the instant invention as, for example, filter element 380. To support, strengthen, and prevent fouling of the fine porosity filter medium, which can be relatively thinner and less structurally stable and resistant to damage and fouling, bigger debris or larger cells can be removed from an air or liquid stream prior to the stream coming into contact with the finer filter element 380. Such an exemplary larger porosity filter can be a 100 micron filter, which can be employed as filter element 370. Such filters are available in a range of pore sizes, ranging from approximately 0.01 microns (micrometer, or 10⁻⁶ meters, 1,000 millimeters) up to approximately 200 microns and larger. Filters may be used alone or in combination with other filters. Filters are or may be available as hybrid filters that combine various filter materials or pore sizes. Alternatively, a filter may have a gradient of pore size, from relatively large to relatively small pores, for example.

In addition to pore size, filters are also available in a plurality of materials. Filters can be made of one or a combination of many different materials such as, for example, glass, polypropylene, polyvinyl chloride, polycarbonate, polytetrafluoroethylene, polyvinylidiene fluoride, mixed cellulose esters, polyether sulfone, nylon, or the like. It should be understood that many potential materials are not listed here, since there are many extant polymers suitable for the task and new polymeric materials are developed regularly. The housing material for the filter could be constructed of materials such as high-density polyethylene, polypropylene, polystyrene, polyvinyl chloride, acrylics, modified acrylics, acrylonitrile-butadiene-styrene polymers, styrene-acrylonitrile polymers, polycarbonate, polyethylene terephthalate, polyesters, stainless steel, or other materials compatible with the needs of filter elements 370, 380.

Pore size, filter material, and housing material are chosen by several criteria, such as compatibility with reagents and chemicals or filter performance under an anticipated range of temperature, pressure, pH, or the like. Other factors that affect the choice of filter include the material to be filtered, the volume or mass to be filtered, and other physical and chemical properties of the filter, filtrate, or effluent that may be understood by those with skill in the art. Regardless of the particular filter material, pore size, and housing material that are appropriate for a given application, filters are commercially available from several vendors including Millipore of Bedford, Mass. USA and Porex Corporation of Fairburn, Ga., USA. Although only two filter elements are illustrated in the various figures and accompanying description, the instant invention contemplates use of one, two, three, four, or more such filter elements that may be stacked or otherwise formed as a substantially integral element having staged or stacked and varying respective porosities through the combined filter, or to have a linearly varying porosity that decreases as the fluid stream moves through the filter arrangement.

Although the various figures, illustrations, and descriptions are directed to embodiments, variations, and modifications of the exemplary configurations of the cell and tissue device 100 wherein the filtration and gas valvule 320 and fluid transfer port 220 are generally positioned proximate the superior portion of the anterior shell 110, those skilled in the art should also appreciate that many other alternative arrangements are possible. For purposes of further examples but not for purposes of limitation, the instant cell and tissue culture device 100 is also susceptible to configurations wherein the fluid transfer port 220 and the filtration and gas valvule 320 can be collocated proximate to and or integrally formed with one another and or formed in and or along any of the peripheral lateral and longitudinal edges 120, 125, 130, 135, 150, 155, 160, 165. In yet other alternative arrangements, the port 220 and the valvule 320 may be configured to be geometrically opposed at opposite corners wherein the port 220 is proximate to the intersection of the superior dextral peripheral edges and the valvule 320 is proximate to the intersection of the inferior and sinistral peripheral edges. The port 220 and the valvule 320 may also be positioned about opposite sides (e.g., the port 200 may be formed in the anterior shell or wall 140 and the valvule 320 may be formed in the posterior shell or wall 110). Those skilled in the area of technology contemplated herein should further comprehend that any combination and similar arrangements are also possible and are compatible for purposes of practicing the instant invention.

Another such alternative arrangement is contemplated as also mentioned elsewhere herein wherein the port 220 and the valvule 320 are formed about one or more of the peripheral edges 120, 125, 130, 135, 150, 155, 160, 165 instead of about the anterior and or posterior external sides of the shells or walls 110, 140 as reflected in the various figures. In variations wherein the port 220 and the valvule 320 are collocated, they can be configured for use with a specialized pipetter that can be configured to facilitate simultaneous infusion and aspiration media concurrent with venting of pressure and vacuum so as to enable the capability for a closed-loop operation, which can be especially useful for purposes of dangerous substances and cell cultures such as deadly viruses and the like. Such a specialized pipetter and cell and tissue culture device, which can be similar in many respects to the device 100 disclosed herein, can be further modified wherein the pipetter is configured with the filtration elements and functional elements that can replace the septum 230 (which filtration and septum elements themselves can be further reconfigurable and replaceable), which pipetter arrangement can simplify the proposed cell and tissue culture device such as device 100 even further for certain specialized applications. Additionally, although not depicted in the figures those skilled in the relevant arts of releasable intravenous and catheter locking needles and “Y-port” type septums, and interconnecting needleless and pipetter tip to septum releasable engagement devices may further understand that the proposed cell and tissue culture device 100 also contemplates adaptability with bayonet-type twist locking and similarly configured interlocking connectors and devices whereby a pipetter such as that described herein or an automated injection, aspiration, assay, and venting system can employ a connector and tip having features and elements adapted to releasably interlock with corresponding features and elements incorporated into the fluid transfer port 220 and the valvule 320 described herein.

With continued reference to the various figures and now also to FIGS. 19, 20, 21, 22, 23, 24, and 25, various aspects of the operation of the cell and tissue culture vessel and flask 100 can be further illustrated. With reference now to FIG. 20 in particular, it can be understood that the treatment of interior surfaces 115, 145 by the contemplated protein treatment 175 or other treatment such as corona discharge and plasma treatment can be applied over the entire interior surfaces 115, 145 or to cover only selected portions thereof. The selective covering or treatment can be accomplished by use of masks or templates that can serve to limit application of such treatments so as to promote cell and tissue adherent growth proximate to the selected portions of the surfaces 115, 145. This approach can be useful in applications suited to simulate and to be compatible for use with processing and analysis equipment designed, for purposes if example, specifically for multiple well plate culturing techniques. As further described herein, various indicia can be imprinted to further improve usefulness of such a limited surface treatment approach, such as indicia to indicate pseudo-well positions or grid locations, as can be understood with further reference to the more detailed descriptions set forth elsewhere herein.

FIGS. 19 through 22 also further described relative preferred placements of various components of one possible configuration of the flask or vessel 100 already described including, for example, the fluid transfer, aspiration and injection/infusion port 220 and the filtration and gas valvule 320 as shown, among other figures, in various disassembled arrangements in FIGS. 2 through 7. Also, with specific reference to FIGS. 21 and 22, the fluid labyrinth configuration having fluid pathways 350, 355 is also depicted in detail in the context of the assembled cell and tissue culture flask or vessel 100. In FIGS. 23 and 24, those skilled in the art may recognize various the operation of any of the preceding embodiments of the cell and tissue culture flask or vessel 100 is compatible with a number of types of pipetters “P” (e.g., FIG. 23) and other fluid (liquid and gas) transfer devices “F” that can incorporate various pipette and pipetter tips T and needleless connectors T′ that can be adapted with a cannula C for piercing and engaging the septum 230. In this arrangement, such pipetters P and fluid transfer devices F can infuse and aspirate gas and liquid to and from the cistern 170 of the flask or vessel 100. A further breakaway detail section view is illustrated in FIG. 25 wherein the septum 230 has been removed to illustrate a fluid pathway denoted generally by arrow labeled with reference letter “A”. Although manually operable pipetter P and fluid transfer device F are reflected in the various figures, the cell and tissue culture device 100 is compatible with and contemplated for use with the wide range of automated processing and handling equipment described herein, which can easily be adapted for use with the flasks or vessels 100 according to the instant invention. This compatibility is especially evident with continued reference to the various illustrations and as further described herein below.

With reference now also to FIGS. 26, 27, and 28, use of the fluid transfer, aspiration, and injection or infusion port 220 is described in more detail. In FIG. 26, the pipetter tip or needleless connector T′ (which can be either manually or automatically operable) and downwardly projecting cannula C (which can be either manually or automatically operable) is shown positioned superior and proximate to septum 230 prior to engagement and piercing. Next, in FIG. 27, the cannula C is pressed into engagement with the septum 230 and slit 235, which forces the septum 230 and slit 235 to deform in response. As the septum 230 begins to deform and the slit 235 begins to received the cannula C, any fluid present in the fluid transfer port aspiration well 250 is forced to follow fluid pathway denoted by the arrow labeled A into lumens 285 and 290. Then, as depicted in FIG. 28, when the cannula C is fully received in and engaged with the septum 230 and slit 235, fluid may be communicated between the tip T′ of pipetter P and or fluid transfer device F and the interior reservoir, chamber, or cistern 170.

In FIG. 29, the cell and tissue culture flask or vessel 100 is illustrated with some hidden edges shown as dashed lines for purposes of additional illustration of the operation of the flask 100. More specifically, those skilled in the art may recognize the configuration of the vessel or flask 100 that incorporates the lumen forming channel 290 with the distal lumen port 300 positioned to communicate fluid between the fluid aspiration infusion transfer port 290 and the interior cistern or chamber 170. Once the cell and tissue culture device or flask 100 has been employed to culture cells or tissues and the need arises to selectively remove either the entire contents or the cells or tissues, or the media or by-products of such a culture, the media contained within the vessel or device 100 may be subjected to enzymatic treatments and or tapping and centrifugation techniques to release and or pellet the cells and tissues. Those having skill in the relevant arts customarily, among other related terminology, use the terms pellet and pelleting to refer to the aggregation and sedimentation of cells, tissues, organelles, and other constituents and by-products and media components contained within a cell and tissue culture device by centrifugation and other sedimentation techniques. As described herein, such cells and tissues may be removed in variations of the preferred embodiments of the vessel or task 100 by removal of the optionally incorporated respirator 180. Additionally, if such cells and tissues are to be removed via the fluid transfer port 220, then additional capabilities of the vessel or flask 100 may be utilized.

With reference next also to FIGS. 30 and 31, those having familiarity with the relevant technology of the instant invention will appreciate that, if necessary for adherent cell lines or tissues, the cell and tissue culture flask or vessel 100 may be subjected to mechanical release techniques and or enzymatic release agents that can be infused into media M that will be contained in the cistern or chamber 170. For purposes of illustration but not limitation, the lumen forming channel 290 is depicted in a schematic representation in FIGS. 30 and 31 and is further annotated generally with fluid path direction arrows labeled 0 and 0′ to denote infusion and aspiration fluid flow pathways. Next, the device or flask may be subjected to tapping and or centrifugation forces to facilitate the process of sedimentation or pelletization of the cells or tissues into a pelleted mass P about the inferior portion of the chamber or cistern 170.

By orientating the cell and tissue culture flask 100 generally as reflected in FIG. 30 during the pelletization process, those skilled in the art can appreciate that the cell or tissue mass can be pelleted substantially proximate to the dextral lower portion of the cistern or chamber 170 closest to port 300 to facilitate the aspiration of the highest concentration of such cells and tissues. As such cells and tissues are aspirated, the upper fluid level surface F of the media contained in the cistern 170 will drop and can be observed through the possibly transparent walls or shells 110, 140 of the vessel or flask 100. In contrast, such skilled individuals can further comprehend that orientation of the cell and tissue flask 100 generally as depicted in FIG. 31 during the pelletization process will facilitate aspiration of the media and culture by products having the lowest concentration of such cells and tissues since the pellet P is concentrated primarily about the sinistral lower portion of cistern or chamber 170.

With these capabilities in mind, those having the requisite knowledge of the related arts may further appreciate and come to understand that the instant invention establishes a new and novel means by which users and operators can perform differential selective centrifugation that can enable discriminatory aspiration of the desired harvest target or product of the cell and tissue cultures contemplated by the instant cell and tissue culture flask or vessel 100. This differential selective centrifugation capability enables researchers, scientists, and commercial operators and users alike to inoculate, incubate, store, transport, inspect and analyze, elutriate, centrifuge, and harvest the contemplated cell and tissue materials all in one, single container. The cell and tissue culture flask described herein in its myriad or multitudinous variations and alternative configurations establishes a high-density capable, highly optimized and media efficient, and compartmentalized and sterile environment that is easily accessed and from which the harvest target can be obtained without damage to the target either by mechanical harvesting techniques (scraping) or by exposure to contaminants or non-sterile environmental atmosphere, instruments, or equipment.

With continued reference to the preceding preferred embodiments, options, modifications, variations, and alternatives as illustrated above and in the accompanying figures, reference is now also made to FIGS. 32, 33, 34, 35, and 36, wherein further details of the proposed optional siphon lock alternative configurations are explicated. More specifically, as diagrammatically represented in FIG. 32, the preferred cell and tissue culture device 100 is depicted in a wire-frame dashed line representation that outlines the cistern or chamber 170, which is surrounding by another embodiment of the previously discussed fluid labyrinth. As noted herein, during unusual attitudes and when subject to certain gravitational and loading or force profiles, such as those imposed upon the vessel or flask 100 during transportation and handling, the possibility may exist that the resulting internal head pressure of the cell, tissue, and media contained in the cistern or vessel 170 may be sufficiently high so as to create unusually high pressures at the fluid transfer port 220 and or the filtration and gas valvule 320.

Those knowledgeable in the relevant arts customarily use the term or phrase “head pressure” in various contexts to refer to the mechanical force per unit area that is exerted by a liquid or gas on an object or a surface, where the force acts at right angles to the object or surface and equally in all directions. In the United States, pressure is usually measured in pounds per square inch (PSI). In other international usages, pressure is defined in terms of kilograms per square centimeters, or in atmospheres, or in Newton per square meter. Also, in various scientific and medical applications, pressure is defined as a relative unit of measure that is typically compared to the pressure of 1 “atmosphere” at sea level and at a standard temperature wherein 1 atmosphere exerts about 14.7 pounds per square inch or about the same pressure developed upon a surface that supports a column of mercury at about zero degree Celsius (32° F.) equal to about 29.92 inches or about 760 millimeters (also referred to as 760 torr) in height, which is about 1033.2 grams (force) per square centimeter.

In the context of the instant invention, those skilled in the art can understand that the head pressure exerted upon the fluid transfer port 220 and the filtration and gas valvule 320 is at a maximum when the flask or vessel 100 is generally inverted relative to the gravity plane. In other words the pressure or force per unit area that is developed by the column of cells, tissues, and media M contained in the cistern or chamber 170 of the flask or vessel 100 is at a maximum when the port 220 and or the valvule 320 is at a the lowest point relative to the inferior edges 125, 155 when such inferior edges are generally directed upwards relative to the gravity plane. In still other words, relative to FIGS. 30 and 31, the maximum pressure of the column of cells and media M contained in the cistern or chamber 170 will be established when the flask or vessel 100 is inverted to that of the noted figures. Additionally, the head pressure developed in this orientation may be further increased as a result of various other forces imposed on the flask or vessel 100 during handling and transportation. With these considerations in mind, those skilled in the art may further appreciate the benefit of incorporating the above-mentioned siphon lock capabilities into any of the preceding embodiments, modifications, variations, and alternatives of the contemplated flask or vessel 100 described herein.

Generally, during operation of aspirating and infusing media and other liquids from and into the cistern or chamber 170 via fluid transfer port 220, the respective vacuum or pressure that develops as a result is vented via the fluid labyrinth and through filtration and gas valvule 320. However, over-infusion or undesirable orientation of the flask or vessel 100 during infusion can interfere with optimal operation of the valvule 320 wherein liquid media and the like can impinge upon the generally fluid impervious components and elements of the valvule 320 in a manner that interferes with the venting or relief capability of the filtration and gas valvule 320 as well as with the most desired operational capabilities of the fluid transfer port 220.

Although the preferred arrangement of components and materials to be employed in fabricating the valvule 320 and the port 220 are preferably selected and adapted to avoid leakage in most applications, various environments and applications may warrant improved techniques to minimize or eliminate the likelihood of any such leaks. To overcome such possible detrimental effects and to the minimize the head pressure that may be exerted against valvule 320 and port 220 assemblies, the flask or vessel 100 of the instant invention may incorporate the above-mentioned siphon lock lumen arrangement, among many other possible configurations adapted to address unlikely but possible pressure and leak issues.

With reference specifically now also to FIG. 32, one such proposed siphon lock arrangement is depicted wherein the fluid labyrinth further incorporates an extended lumen such as lumen 470 having gas pathways B and bends 480, 482, 484, 486, and 488 that are adapted to have one or more segments that will remain outside of the envelope of the cistern or chamber 170 in all possible orientations of the flask or vessel 100. While the infusion and aspiration of liquid media, cells, and tissues to and from the cistern or chamber 170 is generally accomplished with the orientation of the device generally as shown in FIG. 30 wherein any gas pocket, denoted generally in this figure by reference letter G, is proximate to the filtration and gas valvule 320. The fluid pathways denoted in the various figures by reference numeral 350 (namely, e.g., FIGS. 4, 5, 6, 7, 21, and 22) are also schematically represented in FIG. 32 and are shown to be in fluid communication with siphon lock lumen 470. The “extra-envelope” or outside the envelope (i.e. outside the envelope of the cistern or chamber 170) configuration of the lumen 470 guarantees that the flask or vessel 100 incorporates one or more bends 472, 474, 476, 478 of 90 degrees or arc or more, which bends connect one or more segments 480, 482, 484, 486, 488, one or more of which bends and segments that are preferably arranged to be outside of the 3 dimensional envelope inscribed by the interior surfaces 115, 145 of the cistern or chamber 170. Having such bends and or lumens arranged to be outside the envelope ensures that even if any liquid is undesirably introduced into the lumen 470 during operation, handling, transportation, or storage, at least one bend 472, 474, 476, 478 and or segment 480, 482, 484, 486, 488, will always and in all orientations of flask or vessel 100 remain outside the highest liquid surface of the contents or media M that is contained within the cistern or chamber 170 at any given point in time.

In operation, during infusion of media and other elements into the cistern or chamber 170, any gas contained therein is vented to relieve pressure buildup via the filtration and gas valvule 320 via the instant lumen 470 in the fluid path directions generally indicated by reference arrows B into the external atmosphere E. By utilization of the most preferable pore size of the filtration elements 370, 380 of the valvule 320 that is preferably approximately 0.2 microns, it can be predicted with certainty that only sterile air is vented from the cistern or chamber 170 to the external atmosphere E. Similarly, during aspiration of media, cells, and tissues from the cistern or chamber 170, only clean and sterile external atmospheric air is vented through the valvule 320 and into the cistern or chamber 170 to relieve any vacuum that may develop. Thus, it is apparent that the preferred embodiments and all variations thereof can be used and operated in any environment without regard for the availability of sterile air that can be used to relieve pressure and vacuum within the cistern or chamber 170. This can be especially useful for applications involving use in austere, harsh, and extreme environments, and for application involving biologically hazardous substances, materiel, cells, and tissues that must be contained within the confines of the cistern or chamber 170 without concern about leakage or escape into the external atmosphere E.

Of the many benefits attained by incorporation of one or more of bends 472, 474, 476, 478 and segments 480, 482, 484, 486, 488, those skilled in the art can comprehend that any liquid that may enter the lumen 470 and the fluid pathways 350, 355, can be trapped in any one of the one or more bends 472, 474, 476, 478 and segments 480, 482, 484, 486, 488 during movement and manipulation of the flask or vessel 100, which trapping will minimize if not completely eliminate undesirable head pressure from developing and exerting force upon the filtration and gas valvule 320. Such trapping and pressure minimization effect will be further amplified where liquid is trapped in one or more parallel segments 480, 482, 484, 486, 488 by the contemplated siphon effect established when liquid columns are present in parallel segments 480, 482, 484, 486, 488 that are connected by one or more common bends 472, 474, 476, 478 such that the force of gravity acts equally on each of such substantially parallel columns of liquid that, in turn, share a common gas head space under a vacuum since they are connected via a lumen defined by the common or shared bend.

Thus, in other words, the liquid columns thereby hydrostatically balance one another such that there is no actual siphoning of liquid from one segment to another. The vacuum that develops in the common and shared head space above each of the contemplated liquid columns is also known to those skilled in the art as the capillary effect, which effect is commonly employed by technicians using open-ended pipettes to transfer liquids. More specifically, the technician customarily lowers an end of the pipette into a liquid and then seals, caps, or plugs the upper open end with a thumb or forefinger and removes the pipette from the liquid, which thereby creates a vacuum in the head space above the liquid column in the sealed upper end of the pipette. Atmospheric pressure acting on the lower surface of the liquid column at the lower end of the pipette acts to keep the liquid from escaping the pipette until the upper end is unsealed by the technician. Additionally, this capillary effect or technique is further enhanced in pipettes having interior diameters that are small enough such that the capillary attraction forces of adhesion between the sidewalls of the interior surface of the pipette and the liquid contained therein act in concert with the internal cohesive forces acting between molecules of the liquid to retain the liquid within the pipette.

With these principles in mind, those with knowledge in the relevant fields can understand that the contemplated siphon lock configuration contemplated for use with the cell and tissue culture flask or vessel 100 of the instant invention can readily incorporate such hydrostatically balanced or balancing lumens, such as lumen 470, that can be configured to minimize or eliminate otherwise undesirable internal lumen fluid head pressure that may act upon the filtration and gas valvule 320 and or the fluid transfer port 220. Moreover, by sizing the internal diameters and or dimensions of the lumen 470, additional capillary effect and attraction techniques can be implemented. Various possible lumen configurations are possible that can establish similar capabilities to that contemplated by lumen 470.

In another example of variations, modifications, and alternative configurations of siphon lock capable lumens that are compatible for use in any of the preceding embodiments and optional arrangements, reference is now also made to FIG. 33. In this figure, the cell and tissue culture flask or vessel 100 is shown generally in schematic representation to have the cistern or chamber 170 illustrated by dashed lines. An alternative lumen 490 that incorporates the proposed extra-envelope siphon lock capability is shown in FIG. 33 connected to any of the variations of the filtration and gas valvule 320 and to have at least two bends 492, 494 that connect segments 496, 498 to form multiple traps that operate to trap any liquid that may enter the lumen 490. In this alternatively configured lumen 490, it is possible to reduce, minimize, or even eliminate any head pressure that may otherwise impinge upon the components of the filtration and gas valvule 320 due to the pressure developed from any unexpected column of liquid that may undesirably accumulate proximate to the valvule 320 and during use and operation of the cell and tissue culture flask or vessel 100.

With continued reference to FIG. 33, those skilled in the art may appreciate that a similarly configured outside the envelope, siphon lock arrangement can be implemented to minimize and or eliminate and undesirable head pressure that may develop and impinge against the septum 230 of the fluid transfer port 220. Additional and optional fluid lumen 500 is shown to preferably be in fluid communication with septum 230 to have bends 502 interconnected by lumen segments to communicate fluid to distal lumen port 300, which port 300 has already been described. Although lumen 500 is shown as only having a minimum number of bends 502 that connect various segments 504, 506, those having the relevant expertise may be able to understand that any of the previously described multi-bend, multi-segmented siphon lock lumen configurations are contemplated for use for purposes of minimizing or eliminating the head pressure that may be exerted upon the septum 230 and other components of fluid transfer port 220.

The extra or outside the envelope (of the cistern or chamber 170) operating principles of the siphon lock capabilities described herein can be further exemplified with reference also now to FIGS. 34, 35, and 36 wherein the cell and tissue culture device, flask, or vessel 100 is shown being adapted with various lumens 510 (FIG. 34), 515 (FIG. 35), 520 (FIG. 36), being respectively adapted with bends interconnecting lumen segments that are each adapted with portions arranged to be outside the envelope defined by dashed envelope lines denoted generally by reference letter X. Each of the schematically and or facsimile represented components illustrated in the various FIGS. 34, 35, and 36 are labeled with reference numerals corresponding with the previous described elements, components, and features. Each and all of the various siphon lock configurations are compatible for use in any of a number of possible configurations and combinations with any of the preceding preferred embodiments and as further set forth herein below.

The cell and tissue culture flask or vessel 100 according to the principles of the instant invention also further contemplates configurations that are leak proof and that do not necessarily require any type of siphon lock capabilities. In combination with any of the preceding embodiments and modifications, variations, and optional and preferred alternative configurations, another optional filtration and gas valvule 530 is illustrated in the partial detail view of FIG. 37, which is taken generally about the detail view lines 37-37 of FIG. 19, among any of the other similarly arranged figures, and wherein the flask or vessel is modified to incorporate the proposed optional features of FIG. 37. As with the earlier described filtration and gas valvule 3205 the instant variation incorporates one or more variously configured fluid labyrinth pathways adapted to communicate fluid (air and or liquid) between the interior cistern or chamber 170 and the external atmosphere to equalize and vent any pressure or vacuum during the intended operation of aspirating and infusing fluid (gas or liquid and or particles in suspension) from and to, respectively, the cistern or chamber 170.

In FIG. 37, filtration and gas valvule 530 is contemplated for manual operation and or to be used in conjunction with automatic and automated processing and handling equipment. The valvule 530 incorporates a push-button-type peltate plunger 535 operable to establish fluid communication between the external atmosphere and the internal cistern or chamber 170 through the filtration elements 540, 545 wherein element 540 is analogous to filter element 370 and element 545 is configured similar to filter element 380 already discussed herein, that in combination minimize fouling of the filter elements and maximize filtration capability so as to ensure that only sterile air is communicated between the external atmosphere and the interior cistern or chamber 170 during aspiration and infusion operations.

As preferably configured and operated, the valvule 530 operates to seal the reservoir, chamber, or cistern 170 until actuation of the peltate plunger 535. With further reference also now to FIGS. 38 and 39, those having skill in the instant field of invention may be able to understand that the valvule 520 further can incorporate a means for biasing or urging the peltate plunger 535 to the closed and sealed position reflected in FIG. 38. Although any number of means for biasing can be used, the instant figure depicts, only for purposes of illustration but not limitation, a resilient and generally spherically shaped deformable ball 550 received within an underside recess 555 defined in the underside shield portion of the plunger 535 to capture and center the ball 550. Upon actuation, the peltate plunger 535 is urged generally downward in the direction of the arrow labeled D into a depression space 560 to deform the ball 550. The recess 555 is preferably oversized and adapted to accommodate the deformed shape of the ball 550 as best depicted in FIG. 39.

If needed and or desirable, additional similarly configured deformable means for biasing the plunger 535 generally upwardly can be incorporated as needed, and depending upon the material selected for fabricating the plunger 535. The additional means for biasing or urging (not shown but which can be similar in shape and arrangement to ball 550) can be positioned in similarly defined recesses (not shown but which can be similar in construction to the recess 555) about the outboard edges 565 and in the depression space 560. Also, for purposes of improving the operation of the contemplated alternative valvule 530, a sealing end or ends 570 of the plunger 535 may incorporate anvils 575 that can serve to minimize possibly fouling of the sealing function of the plunger by increasing the point load forces exerted by the anvils 575 against any material that may be present in the labyrinth fluid pathway as the plunger 535 returns to its closed position.

Additionally, the contemplated fluid labyrinth of the instant alternative valvule 520 can, at the inferior ends of wall(s) 585, incorporate anvils 590, which operate to further improve the sealing capability of the modified filtration and gas valvule 520. As a further optional modification to the alternative valvule 520, additional gaskets or seals 580 may be incorporated that can be formed of a low durometer rating elastomeric material that can cooperate with the anvils 575, 590 to even further improve the desired sealing capability. Although not depicted in the various illustrations, the proposed gaskets or seals 580 may be adapted to have a generally looped configuration similar in construction to the elastic bands commonly referred to in many offices and office supply sources as “rubber-bands” whereby a portion of the seals or gaskets 580 are adapted to engage a portion of the opposed and confronting anvils 575, 590 so as to bias or urge them into the closed position reflected best by FIG. 38. Many types of suitable elastomeric and other types of materials are contemplated for use as such gaskets, including for example without limitation, rubber, latex, silicone, synthetic and natural polymeric materials and isoprenes and similarly capable materials, butyls, halogenated butyls, ethylene propylene diene monomoers, nitrites, thermoplastic elastomers, and combinations and mixtures and alloys thereof, to name just possibly suitable materials and substances.

During operation and when the plunger 535 is maintained in the depressed position of FIG. 39, fluid pathways are established through the fluid labyrinth, which pathway is most readily apparent in FIG. 39 and denoted generally by fluid pathway arrows H. In other possibly desirable modifications to the instant filtration and gas valvule 520, a ridge 600 may be incorporated concentrically exterior to the plunger 535 and substantially concentrically interior to the breather ports 330. The ridge 600 is can preferably be adapted with support and stress relief rails 603 to rise above or to the top surface 605 of the plunger 535 so as to prevent unintentional or accidental depression of the plunger 535 during operation, handling, storage, and transportation.

As discussed briefly herein in other contexts, an alternatively and optionally modified cell and tissue culture device 100 that is also compatible with the principles and features of the instant invention further contemplates modifications, variations, and alternative configurations to any of the preceding embodiments wherein the fluid transfer port or ports 220 and the filtration and gas valvule or valvules 320, 520 are in various other possible locations about the flask or vessel 100 than those positions described in connection with the previously illustrated figures and drawings. With reference now also to FIGS. 40 and 41, the cell and tissue culture flask or vessel 100 may optionally incorporate one or more alternative fluid transfer ports 610 and filtration and gas valvules 620 in either (1) the superior and inferior lateral, and or dextral and sinistral longitudinal peripheral anterior and posterior edges 120, 125, 130, 135, 150, 155, 160, 165, or (2) in positions as reflected in FIGS. 40 and 41, wherein the port 610 and the valvule 620 are proximate to one another in a common corner of the flask or vessel 100.

Various additional modifications to the port 610 and the valvule 620 are contemplated by these alternative and optional arrangements and include variations that are compatible with specially configured manual operable and automated system-type pipetter tips and fluid transfer devices. Further, the alternative arrangements of the flask or vessel 100 of FIGS. 40 and 41 illustrate configurations wherein the respirator 180 is relocated and or removed completely from the flask or vessel 100. In the relocated alternative, the respirator can be relocated to be flush against the superior lateral (150) and dextral longitudinal (160) peripheral edges such that when modified to incorporate the pull or peel tabs, such as tabs 186, 186′, or the tear strand or pull wires 188, 188′. The respirator 180 can be removed entirely and the contents of the interior cistern or chamber 170 can be emptied through the aperture 200 as efficiently as possible and without the need to aspirate such contents through any of the contemplated channels and lumens. In non-respirator configurations such as that depicted in FIG. 41, such contents may be aspirated through the fluid transfer port 610.

With reference next to FIGS. 42 and 43, it can be understood that the construction of ports 220, 610 and valvules 320, 520 of any of the preceding embodiments and variations thereof can also alternatively incorporate a differently configured arrangement that can be adapted to be in a side-by-side or functionally analogous concentric arrangement (not shown) that can be adapted for compatibility with the specially configured pipetter tip and fluid transfer device 630. The device 630 can include optional filtration elements 635, 640 that can be similar in construction and capability to those filter elements 370, 380, 540, 545 already described above. In this modification, the filter elements 635, 640 can take the place of the elements otherwise incorporated into the modified valvule 620 of the flask or vessel 100 or can be included to act in concert therewith to establish the sterile communication of fluid, most probably air or gas, between the reservoir or cistern or vessel 170 and the external atmosphere for purposes of venting and equalizing pressure and or vacuum within the cistern 170 relative to the external environment.

The integrally formed device 630 also preferably incorporates cannulae C″ (with needle-type or needleless-type lumens) that is adapted for registration with the pre-slit septum 645 (or other similarly capable aperture) of the port 610 and the septum, also denoted 645 in the figures since it is illustrated as part of the port septum 645, (or other similarly capable aperture) of the valvule 620, and which septums 645 or apertures formed therein function in a similar fashion as already described in detail in the context of other modifications and variations of the various preferred embodiments. Although illustrated as a single component, the septums 645 can be formed as independent elements and of different materials (not shown). Those skilled in the art should be able to discern from these proposed alternative modifications, that the side-by-side or concentric arrangement can operate to simultaneously and or synchronously enable fluid infusion, aspiration, and pressure equalization during manual and or automated use and operations of the proposed cell and tissue culture device according to the various principles and capabilities set forth herein.

Other types of valve configurations that may be possibly desirable for certain applications and that may be compatible for use with the filtration and gas valvule constructions contemplated by the described cell and tissue culture device according to certain aspects of the instant invention include rotating valvule elements similar in construction and design well-known to those having skill in the technical arts of microfluidic valve technology such as the valves illustrated in, for purposes of example but not for purposes of limitation, U.S. Pat. No. 5,586,579 to Diehl and No. 6,293,162 to Mathur, et al., which are each incorporated by reference in their entirety as if fully set forth herein.

In each of the preceding embodiments and the proposed preferred and optional modifications, variations, and alterative arrangements thereto, any of a number of possible configurations of cistern compatible lumen constructions and releasable assembly features can be incorporated to add yet more possibly desirably capabilities to the various constructions of the preferred embodiments. Variations and modifications proposed in FIGS. 44 through 50 include reference numerals that have already been described herein and are intended to describe identical and similarly configured components, elements, and features of previous embodiments and modifications, variations, and alternatives thereto.

With reference next to FIG. 44, which is a partial section view in enlarged scale and rotated that is taken about section lines 44-44 from FIG. 19, it may be understood that the anterior shell or wall 140 may be joined together with the posterior wall or shell 110 by use of a straight splice joint the can readily be fabricated using any number of equally suitable injection molding techniques. The proposed straight splice joint configuration can be joined by ultrasonic and similarly capable welding methods, by adhesive methods, by using various types of fasteners including screws that can be received in preformed holes (not shown), by integrally formed fastening features and elements, and by combinations thereof. As reflected in FIG. 44, the depicted splice joint is formed with a posterior ledge 650 sized to receive an anterior leg 655 and an anterior ledge 660 that receives a posterior leg 665. The anterior leg 655 confronts about one side 658 a corresponding face 668 of the posterior leg. As depicted in FIG. 44, the legs 655, 665 and ledges 650, 660 and the confronting faces 658, 668 can be permanently or releasably joined by adhesives, welding, fasteners and fastening features, and by combinations thereof.

With continued reference to the various figures and also now to FIG. 45, one particular type of preferable and optional alternative configuration of the contemplated splice joint of FIG. 44 can further incorporate a fastening capability. The optionally fastening configuration can include a posterior ledge 670 and recess 675 along an inferior portion of a surface 678 of the posterior wall or shell 110, the ledge 670 and recess 675 being adapted to fixedly or releasably engage with an anterior resiliently bendable or deformable leg 680 that cooperates with another anterior leg 685 having a protruding tip 688, sized for receipt in the recess 675, in a clevis-type arrangement adapted to slide over and capture the ledge 670 and recess 675 in a positive latch manner. The size, shape, and thicknesses of the respective components can be selected to create a snap-fit arrangement that establishes an identifiable and perceivable click and lock tactile feedback to the user during assembly to tactilely communicate that the posterior wall or shell 110 is captured and engaged with the anterior shell or wall 140.

In yet another possible joint configuration, as can be seen with further reference now to FIG. 46, the preferred cell and tissue culture flask or vessel 100 may also be modified to incorporate another type of splice joint that incorporates what are commonly referred to by those skilled in the relevant arts as friction fitting fastener features. More specifically, a splice joint is contemplated that includes the posterior ledge 670 but that does not include the recess 675. Instead of being received into the now absent recess 675, the protruding tip 688 slides along and frictionally grips the surface 678 to create the friction fitting fastener feature.

In any of the preceding embodiments and modified arrangements thereof, various lumens may be formed from channels than can be integrally formed in and fabricated as part of the contemplated fastening release elements. With reference now also to FIG. 47, any of the previously described and contemplated lumens and charnels may be formed as depicted in the instant figure, including, for purposes of example but not for purposes of limitation, the lumen 290 reflected in various drawings including FIGS. 21 and 25 through 31, and the lumens 470, 490, 500, 515, 520 illustrated and contemplated in FIGS. 32 through 36.

More specifically, a single lumen 700 of FIG. 47 may be defined by channel 705 formed between posterior legs 710, 715 and shortened anterior leg 720. Such lumens as lumen 700 can formed to have a variety of possible dimensions and shapes and fluid pathways and can be sized in the context of one of the various embodiments disclosed herein to be between 0.1 and 3.5 millimeters in various dimensions, and more preferably to be between about 0.5 and 2.0 millimeters, and even more preferably to be about 1.0 millimeters in various dimensions. Additionally, the lumen 700 can be configured to create a wide range of possible fluid pathways including as contemplated by the various illustrations, schematics, and diagrammatic representations set forth in connection with lumens 290, 470, 490, 500, 515, 520 in the various drawings and discussions, as well as any of the many other configurations and arrangements contemplated by the instant invention.

As a further illustration and example, a multiple lumen arrangement can be similarly accomplished as described in part in FIG. 48, wherein lumens 725, 727 are formed by respective channels 730, 732 formed between respective posterior legs 735, 737 and anterior legs 740, 742. Additionally possibly desirably configurations of integral lumens and fastener capabilities are illustrated in FIGS. 49 and 50. In FIG. 49, lumens 745, 747 are defined by respective channels 750, 752 formed between posterior legs 755 (dextral), 757 (sinistral) and anterior legs 760 (dextral), 762 (sinistral). With reference to the preceding illustrations, it can be further understand in the context of FIG. 49 that fastening elements such as protruding tips 763 and recesses 764 can be further incorporated into the legs 755, 757, 760, 762 for purposes of establishing the permanent or releasable latching and fastening capability contemplated by the cell and tissue culture flask or vessel 100 of the instant invention. Similar features, components, and elements are illustrated by FIG. 50 in a variation of the configuration depicted in FIG. 49 wherein like reference numerals with primes correspond generally to the reference numerals described in FIG. 49. For example, reference numerals 763′ label the protruding tips of FIG. 50 and numerals 764′ identify the recesses of the same figure, and these numerals correspondence respectively to reference numerals 763 and 764 of FIG. 49, which numerals describe correspondingly similar elements and components.

Although the various configurations of lumens, channels, legs, protruding tips, recesses, and other contemplated fastening means are described primarily in connection with the dextral and sinistral and anterior and posterior longitudinal peripheral edges 130, 135, 160, 165, any and all of such features, elements, and components are compatible for use in connection either alone or in various other combinations with the lateral superior and inferior and posterior and anterior peripheral edges 120, 125, 150, 155 as can be understood with continued reference to the preceding descriptions and illustrations of the various figures.

To further illustrate various possible releasability capabilities described herein, the reader is now invited to also make reference to FIGS. 51A and 52B, which are side elevation views of any side or all sides of the contemplated cell and tissue culture flask or vessel 100 that shows additional variations that are compatible for use either alone or in combination with any of the preceding variations, modifications, and alternatives already described. More specifically, with reference now also to FIG. 51A the cell and tissue culture flask or vessel 100 is depicted being formed with anterior release notches 765 and posterior release notches 767 that are adapted for use with a clam shell type release tool (not shown) that can exert point load release forces in the notches to separate the anterior shell or wall 140 from the posterior shell or wall 110 in way that minimizes or eliminates any damage thereto. In this way, those skilled in the art may come to understand that the releasable embodiments of the preferred cell and tissue culture flask 100 can be especially useful in applications where ordinary aspiration of the cultured cells and tissues from the various lumens, such as lumen 290, or through the releasable respirator 180, 180′ are less desirable than removal by separating the shells 110, 140. Although not shown in the various figures, the instant cell and tissue culture flask or vessel 100 can also further incorporate integrally in the walls or shells 110, 140 scoring lines that can facilitate cracking to separate the inferior portion of the shells or walls from the anterior portion for purposes of removing the contents of the cistern 170. Also, a scoring or cracking tool (not shown) much like a glass cutter can be employed to score the material of the walls or shells 1120, 140 to facilitate such separation of the walls 110, 140 and removal of the cistern 170 contents.

Any of the preceding joining methods described can be adapted for compatibility with the releasability features contemplated herein in various ways that establish predictable separation forces. Such joining methods can also be further configured specifically for use with various types of ultrasonically welded joints such as that illustrated in FIG. 51B. In this figure, those skilled in the arts of polymeric and elastomeric joining techniques may appreciate that the earlier described straight splice joint can be injection molded to have posterior leg 770 and posterior ledge 772 formed with ultrasonic energy concentrators or weld tips 774 that are adapted to melt during welding, such as by ultrasonic welding, with enough precision to create a joint having a reproducibly predictable and generally consistent separation force. The weld tips 774 may run the length of the leg 770 or ledge 772 to form seam-type welds or may be intermittently spaced apart as what may appear to be posts, dimples, or stipples to form spot welds having the same desirable weld and release characteristics.

Although a wide range of suitable shapes and sizes are contemplated herein, the substantially sharp top points of the tips 774 illustrated in the various figures serve to maximize frictional forces and to concentrate the ultrasonically induced energy to enhance efficiency of energy transfer to the tips 774 for fast melting and welding. It has been found that weld tips 774 can be of a wide range of shapes including rails, ridges, dimples, and stipples that are sized to have a height and width of between about 0.05 and 2 millimeters, and more preferably between approximately 0.1 and 1 millimeters and even more preferably in the range of about 0.2 millimeters and 0.5 millimeters are satisfactory for purposes of the instant invention. Also, inside corners 775 and outside corners 776 can be sized to have very small and even sharp corner radii so as to further concentrate such energy to increase the melt region for improved joint strength.

In this way, the resulting welded joint can be separated using the contemplated clam shell type separation tool (not shown but within the skill of those knowledgeable in the relevant art). As can be understood with continued reference to FIG. 51B, the contemplated resulting welds can be spot welds, or circumfluent welds spanning the entire longitudinal and lateral span of the surface shown of leg 770 and or ledge 772. Although shown as being formed on the posterior shell 110, the tips 774 may also be formed only or also on the anterior shell or wall 140, and the tips 774 may only be formed on either the leg 770 or the ledge 772.

With continued reference to FIG. 51B, it can also be observed that in contrast to other embodiments, the illustrated variation of the walls or shells 110, 140 also include interior surfaces 115, 145 that incorporate generous fillets 778 that can improve the cell and tissue culturing capabilities of cistern or reservoir or chamber 170 for certain culture applications. In addition to improving such culture capabilities, the fillets 778 serve to minimize if not even deflect ultrasonic energy accumulation so as to avoid concentrated energy buildup that can lead to undesirably melting or stress load buildup in the heat affected zones for various embodiments of the flask or vessel 100.

In FIG. 52A, alternatively positioned anterior notches 765′ and posterior notches 767′ are depicted, which can operate to establish predictable release forces and which can be formed and arranged in a similar manner to that described in connection with the notches 765, 767 of FIG. 51A for purposes of compatibility with the proposed clam shell-type release tool (not shown). Additionally, in FIG. 52B an alternative arrangement of weld features is shown that includes the weld tips 774 but which also includes energy deflector fillets 780 that can be sized and shaped to adjust and or minimize concentration of ultrasonically induced energy so as to minimize and or prevent ultrasonic welding proximate thereto, which features can be useful to form the predictable joint separation forces discussed herein. Such energy deflector fillets 780 that have been found to be suitable for purposes of the instant invention include fillets having radii approximately between 0.5 and 10 millimeters and more, and more preferably between about 1 and 8 millimeters and even more preferably between about 2 and 5 millimeters. In combination with selection of various types of adhesives, thermoplastics for forming the shells or walls 110, 140 and the size, shape, and arrangement of the splice joint features and elements, a wide range of possible predictable, reasonably precise, and even calibratible separation forces can be established. Further, FIG. 52B also depicts a seam filler 782 that can be selected from a material that can form a sealing gasket, a joint establishing and or strengthening adhesive, or both. Such a seam filler or gasket or adhesive can be employed to ensure a hermetic and or tight seal is established between the walls or shells 110, 140 after assembly and can be formed from a wide range of materials that include, for purposes of illustration but not limitation, rubber, latex, silicone, synthetic and natural polymeric materials and isoprenes and similarly capable materials, butyls, halogenated butyls, ethylene propylene diene monomoers, nitrites, thermoplastic elastomers, and combinations and mixtures and alloys thereof, to name just possibly suitable materials and substances.

Turning now to yet more possibly desirably and optional features and components, in FIG. 53, additionally optional automated and manual handling crenelations 785 are shown that can be adapted for compatibility with various manually operated and automated processing, handling, and storage devices and equipment. Such crenelations 785 can, for purposes of example without limitation, be employed for latching devices similar to those used for computer memory chip and chip set pivot-lock and snap-fit sockets customarily used on and located on microcomputer motherboards, daughter cards, and the like.

Further in FIG. 53, alternative arrangements of any of the preceding embodiments can also incorporate various types of rigidity enhancing and structural integrity improvements and features, such as, for purposes of example but not for purposes of limiting the scope of the many aspects of the instant invention, strengthening ribs 790 can be seen formed in the one or both of the shells or walls 140 and wall or shell 110 (the latter being directly beneath and registered with the shell or wall 140 shown in the plan view of FIG. 53). The ribs 790 may be formed in any number of possible configurations but are shown in one possible such arrangement configured for compatibility with distributing the stresses that may be encountered not only during routine uses, but also that may be developed by the internal column of media, tissues, and cells against the walls 110, 140 and other components of the flask 100 during centrifugation at an orientation compatible for purposes of pelleting the cells, tissues, and other contents of media M so as to establish the desired pellets P, such as those described in connection with FIGS. 30 and 31.

As will be described herein in more detail in connection with the further detailed illustrations of the strengthening ribs 790, anti-crush posts, pads, or elements 792 can be observed with continued reference to FIG. 53. FIG. 54 is a partial cross section containing media M and taken about section lines 54-54 of FIG. 53 for purposes of illustrating a construction of anterior shell 140 and the posterior shell 110 without ribs 790 or anti-crush pads 792 so as to provide a contrasting context for the following discussion of such strengthening ribs 790 and anti-crush posts and pads 792.

In FIGS. 55 through 62, additional and optional automated and manual handling system features are illustrated in combination with various additional and optional flask 100 strengthening components, each and all of which are contemplated and compatible for use either alone or in combination with all of the preceding embodiments, modifications, variations, and alternative arrangements described herein. More specifically, in FIGS. 55 and 56 the cell and tissue culture flask or vessel 100 is shown with the crenelations 785 in combination with lateral anterior and posterior keyways 800 and longitudinal anterior and posterior keyways 805. Although the crenelations 785 and the keyways 800, 805 are shown generally proximate about particular peripheral lateral and longitudinal edges 120, 125, 135, 150, 155, 165, they can be formed in any single or combination of such edges as may be desirable for use in connection with a wide range of possible manual and automated handling and processing devices, equipment, and systems. Additionally, even though the crenelations 783 are shown are depicted for purposes of illustration but not for limitation to be generally formed in opposite peripheral edges, either one or both or more such crenelations 785 may be desirable in given applications. Similarly, even though the contemplated keyways 800, 805 have been found to be effective in the configuration wherein they are formed about opposite anterior shell 140 and the posterior shell 110, certain applications are well-suited to forming the keyways 800, 805 about only a single side of anterior shell 140 or posterior shell 110. For purposes of describing exemplary and possibly desirable and preferably modifications to any of the embodiments described herein, but not for purposes of limitation, FIG. 56 is illustrated to incorporate the flask 100 construction most closely similar to that depicted in connection with FIGS. 21, 26, 27, and 28.

With continued reference to FIG. 55 and the partial cross-sectional views of FIGS. 57 through 62, various possible constructions of stress relief and distribution ribs 790 are illustrated in more detail, which ribs are shown in one of many possibly preferably arrangements that are best suited for strengthening the shells 110, 140 and for distributing and relieving operational stresses encountered during routine use, processing, handling, storage, transportation, and centrifugation. In FIG. 57, the rib 790 is formed in only one of the shells or walls, namely the posterior shell or wall 110. In contrast to the construction of FIG. 57, FIG. 58 reflects an alternative configuration also taken figuratively about section line 57-57 of FIG. 55 wherein strengthening and stress distribution and relief ribs 790 are formed into both the anterior shell 140 and the posterior shell 110.

In FIG. 59, another possible arrangement of stress distribution and relief ribs 790 are illustrated for purposes of example without limitation wherein the ribs 790 are shown to be formed tangent to and separate and apart from the anti-crush pad 792. Further in FIG. 59, the anti-crush post or pad 792 is shown in one of many possibly suitable and equally effective configurations wherein the pad 792 is integrally formed as part of one of the shells or walls 110, 140 and projecting against and centered about a recess 794 formed in the end of the pad or post 792. In configurations wherein the contemplated rib 790 does not extend fully past the post or pad 792, such as shown in the configuration of rib 790′ of FIG. 59, a centering rib or dimple 796 can be formed in the respective shell or wall 110, which dimple or rib 796 that can be received against the recess 794 may also further be formed integrally with either of the corresponding walls or shells 110, 140 and formed independently of the other types of contemplated ribs 790.

In FIG. 60, which is a representative and possible partial cross-sectional view that could have been taken about section line 59-59 of FIG. 55, another possible configuration of the anti-crush pads or posts 792 is shown wherein the pad or post 792′ is formed independently of either wall or shell 110, 140 top have multiple recesses 794 that can be received against ribs 790 and or dimples or centering ribs 796. The illustration of FIG. 61 reflects another possible arrangement of the contemplated ribs 790, pads and posts 792 with recess 794 received against the rib 790 or the dimple 796 and wherein the pads or posts 792, 792″ are matingly registered against one another after assembly of shells or walls 110, 140. In FIG. 62, yet another possibly preferable and optional modification is depicted wherein the contemplated pads and posts 792, 792″ of FIG. 61 are further alternatively reconfigured to include, among other changes, interlocking snap-fit post 797 and recess 798 which can be sized, shaped, and adapted to have a predetermined release force and to be compatible for use in connection with either the permanently attached or the releasable configurations of cell and tissue culture flask or vessel 100.

Turning next also to FIGS. 63 and 64, the instant invention also can be optionally modified with respect to any of the embodiments, variations, and alternative described and contemplated herein to include various additional indicia that can be configured to augment various operational requirements. One such requirement can include, for purposes of explication but not limitation, various information labels that serve to assist the operator in identifying various components of the cell and tissue culture device including the fluid transfer port 220, which label can be “PIPETTE TIP” (FIG. 63) and the filtration and gas valvule 320, which can be labeled “I/O AIR” (FIG. 63) for input and output of sterile air. For various applications, the exit port 300 of the fluid communication lumen 290 of the fluid transfer port 220 can be labeled “I/O MEDIA” (FIG. 63) so that the operator or technician can observe infusion and aspiration of substances to and from the cistern 170 without having to visually locate such port 300, which can be very small and difficult to perceive by the unaided human eye.

In the illustrations of FIGS. 63 and 64, the exemplary indicia, which are depicted for purposes of example but not limitation, can be formed on either the interior or exterior of the anterior shell or wall 140 or the posterior wall or shell 110, or combinations thereof, and can be included on the cell and tissue culture flask or vessel 100 whether or not the respirator 180, 180′ is incorporated in the particular embodiment of interest. Such indicia may be formed integrally as part of the material used to fabricate the walls or shells 110, 140, or may be imprinted thereon using any number of equally effective methods. Combinations of imprinting and integral fabrication are also possible and can further incorporate techniques for maximizing the contrast between the indicia and the surrounding portion of the flask or vessel 100 and the contents of reservoir, chamber, or cistern 170 and the shells or walls 110, 140. For further example, such indicia can be integrally formed and then be subject to application of high-contrast substance such as a white or other high-contrast material that can amplify the contrast for improved imaging characteristics. Similarly, imprinted indicia can be imprinted in multiple layers on the interior or the exterior, or both of the shells or walls 110, 140 wherein the line segments of such indicia can be of one of a plurality highly-contrasting colors or pigments or substances, and one or more other layers can be of another of the plurality of highly-contrasting colors of pigments or other substances. Such colors or pigments or similar substances can be selected for compatibility with the contemplated contents of the flask or vessel 100 such as to include only those materials that preferably inert when exposed to such contemplated contents.

In general and depending upon the particular application, such exemplary indicia may be formed with the smallest of lettering, numbering, and line widths for a number of desirable applications including, for purposes of example without limitation, 1) minimizing any possible obscuration of the contents of the cistern 170, 2) being compatible for use and for legibility for videoscopic and microscopic visualization and imaging applications, 3) being compatible for use with a variety of automated imaging systems having autofocus and autosharpening capabilities and automated histological, cytological, taxonomic, and similarly capable systems, to name just several examples. Such exemplary indicia may also be formed from substantially optically transparent materials and in a visually unobstructing manner so as to maximize the capability of flask or vessel 100 for optimized viewing of the contents of cistern, chamber, or reservoir 170 and any materials adhered to surfaces 115, 145.

Also, all indicia, including lines, letters, and numbers, may be formed to have a precisely predetermined lengths, widths, and other similar features and elements that may be used for purposes of calibrating visually perceived indicia and the contents of the flask or vessel 100 so as to establish reference dimensions and to minimize or eliminate calibration errors, which calibration and reference data can be utilized by imaging techniques to further augment efficiency and capability of the flask 100 in processing, recording, and analyzing the details of interest for the contents of cistern 170.

Such automated imaging analysis devices and apparatus can utilize such indicia for automatic dimensional calibration of any such images obtained of the contents of cistern 170. In yet other possibly desirable configurations, any or all such indicia may be sized for viewing by the unaided human eye, by macroscopic visualization equipment, and by microscopic visualization apparatus. More specifically, such systems can often employ, among many other possible configurations, a magnification capability that can enlarge the visually perceived information by 10 times its actual size for initial macroscopic observation, image centering, and for automated imaging systems—imaging focus and sharpening purposes. Such preliminary or initial magnification can also be accomplished at smaller and greater magnifications. Once the image region has been identified, centered, focused, and sharpened within the bounds of the visualization window for which an image is to be captured, an additional magnification capability of 20 times, 40 times, or more, or less, is employed for purposes of detailed analysis, image capture, and manual or automated data processing.

With the 10 times magnification example, the customary and long-used manual objective and optics of the visualization equipment contemplated hereby typically render a macroscopic and circular field of view having a diameter of about 2 millimeters or 2000 microns (“μm”). After image centering, orientation, focusing, and sharpening, the higher magnification(s) are typically employed, which reduces the field of view correspondingly. More modern and present imaging systems have expanded upon and augmented this original configuration and now typically employ imaging devices that include videoscopy capable equipment that can incorporate, among other elements and capabilities, charged-coupled devices or “CCD” semiconductor chip-set arrays of imaging devices or cameras. In the early development of such imaging devices, they initially had a rectangular 2-dimensional field of view (with additional dimensions of color, luminance, etc.) of about 800 pixels wide by about 600 pixels in height wherein about 700 pixels could be calibrated to have a real world resolution of about 2 millimeters at a magnification of 10 times, which corresponds to the classical field of view of a 10 times magnification manual imaging objective. As already noted, the field of view decreases corresponding to the possibly desirably higher magnifications. Presently, the most widely available CCD imagers are capable of square fields of view of about 1,024 pixels, or more ore less depending upon the technology and the source supplier, in two mutually orthogonal dimensions with a range of possible resolutions that depend upon the optical capabilities of the objective lens arrangement.

With respect to the classical circular field of view, the newer solid state CCD square fields of view typically inscribe a rectangle or square within the circular field of view presented to the CCD image plane from the objective optics. The inscribed square focal plane of the CCD array can, at the 10 times magnification, typically capture and render an image in the range of about 1.4 to 1.5 millimeters on a side, within the diametrically 2 millimeter circular field of view rendered by the objective lens arrangement In addition to using low and high power optics to obtain the image of the subject matter of interest, such CCD devices are also typically used in conjunction with various types of microprocessor and computer-based imaging hardware and software that can automatically acquire an image and sharpen and focus the image by various techniques, which can involve the recognition of an interface line that is defined by one or more dark and light regions of the acquired image. These techniques work best when the contrast between the dark and light areas of the image are sharp, that is to say they work best in achieving optimum automated focus and sharpening when the interface is between a black region and a white region of the acquired image.

With these considerations in mind and in the context of the many embodiments and alternative configurations of the devices according to the principles of the instant invention, those having knowledge in the technological field can appreciate that the indicia set forth and illustrated in the description and figures herein can have black and white adjacent elements to facilitate optimum focusing and resolution calibrating capabilities. Moreover, for compatibility with the most commonly employed printing, lithography, and other publication computer hardware and software, it has been found that certain minimum dimensions of features of images to be reproduced thereby should most optimally have lateral widths of approximately between 0.05 and 1 millimeters, and more preferably in range of about 0.09 millimeters and 0.5 millimeters, and even more preferably approximately 0.2 millimeters. In other words, when the line widths are about 0.2 millimeters for any indicia to be used for purposes of the instant invention, such indicia can be especially well-suited to certain reproduction and printing applications that transfer the proposed indicia onto the substrate forming the flask or vessel 100. Many other possible configurations and sizes of indicia are also contemplated that are compatible for various other applications.

With continued specific reference to FIG. 62, location and position indicia 810 can implement a reference grid with rows and columns to facilitate analysis and inspection of the contents of cistern 170 and any cells or tissues that may be adhered to the surfaces 115, 145. As shown in FIG. 63, a reference grid format is shown that may be compatible with a range of 96-well or microtiter place analysis systems that are adapted to view, analyze, and even record the contents of such 96-well and microtiter plates. The position indicia 810 are shown in FIG. 63 as being generally adapted for viewing by the unaided human eye. In addition, although not shown in the figures, the position indicia 810 may be further reproduced in microscopic scale so it is readable by manual and automated microscopic visualization equipment. Similarly arranged and applicable location and position indicia 810′ are shown in FIG. 64.

In addition to the image calibration line widths described in connection with position indicia 810, 810′, center point target and visualization calibration indicia 812 and periphery target and visualization calibration indicia 814 are shown in FIG. 63. For purposes of manually or automatically calibrating the visual image perceived of the contents of the cistern 170 of the flask 100, and in addition to the possibly predetermined line widths already noted herein, the target and visualization calibration indicia 812, 814 can also be formed to have a predetermined length of, for example without limitation, 10 millimeters, or any other suitable predetermined length that may be compatible for use with various automated image analysis systems. Further, the center point target and visualization calibration indicia 812 may also be formed to have a preselected shape or to inscribe a predetermined pattern of lines that can include horizontal, vertical, and angled lines of predetermined widths and lengths and relative angles, all of which data can be preprogrammed into various types of imaging analysis systems and software so as to augment the possible image calibration accuracy that would otherwise possible. Although the center point target and calibration indicia 812 of FIG. 64 is configured as a straightforward crosshair with 10 millimeter long line segments and a center registered circle of about 2 millimeters in diameter (which corresponds to the classical 10 times enlargement field of view most commonly in use in general visualization and imaging applications), as well as a more complicated center point and target indicia 812′ (FIG. 64) is also well-suited for purposes of the instant invention. Each or any of such indicia 812, 812′, 814 can be set to have predetermined and calibrated dimensions in units of microns wherein the line width can be approximately 0.2 μm and the line segment lengths can be, for example purposes without limitation, 1, 10, 15, and 20 millimeters in length, and longer and shorter, which can be used with a digitized image to calibrate to the image and the rendered image to real world dimensions. The more complicated configurations of such indicia, such indicia 812′, can include variously angled lines so as to establish a known real world dimension about one or more axes in the plane of the acquired image whereby the user can manually or in an automated manner calibrate the pixel to real world dimensions preferably optimized accuracy so as to establish a useful scale and calibration capability for enabling accurate visualization and analysis of the cells and tissues and structures thereof that may be contained within cistern 170 and on the surfaces 115, 145.

These proposed arrangements and configurations of the visualization indicia 812, 812′, 814 are especially effective with various manual and automated image and visualization calibration techniques that are adapted minimize lensatic related aberrations across image plane, and with the appropriately configured imaging systems and software, can effectively overcome possible keystone effects due to any misalignment between the image acquisition device and the plane of surfaces 115, 145. Many existing automated software and hardware based image analysis systems are particularly well-suited for adaptation to use the inventive cell and tissue culture vessel 100 in a large number of applications that include, for example without limitation, histology, cytology, taxology methods including “TICAS” or taxonomic intracellular system classification, auto harvest and stain systems, as well as a wide range of imaging, cataloging, high-density cell culture quality assurance, and incubation, handling, sampling, processing, analysis, and storage systems.

Continued reference to FIGS. 63 and 64 further illustrates various calibrated volumetric indicia that can be of use in various applications of the preferred and modified embodiments of the cell and tissue culture vessel 100 of the instant invention. This is a pronounced benefit in particular with respect to the new and inventive minimum static media (“MSM”) approach that is newly established for purposes of optimizing use of the preferred and optionally modified embodiments of the cell and tissue culture flask 100. More specifically, volumetric indicia 816 of FIG. 63 can be incorporated and which can assist a user of the vessel or flask 100 in infusing substances and materials into the vessel 100. Further, such volumetric data can further identify the internal surface area available of the particular flask or vessel 100, as well as the volume per unit area thereof. As can be further understood from FIG. 63, such volumetric indicia 816 can be integrated with other indicia, such as position and location indicia 810, 810′, so as to minimize obscuration of the contents of the surfaces 115, 145 and the cistern, reservoir, or chamber 170 when desirable.

In FIG. 64 various optionally preferably and enhanced volumetric indicia 816′ and 818 are shown. In this possibly desirable configuration, the calibrated volumetric indicia 816′ are not integrally formed with the position and location indicia 810′. Further, volumetric indicia 818 also incorporate angled liquid level lines that can be useful during infusion and aspiration of pellet concentrations as contemplated in the operational descriptions set forth in connection with FIGS. 20 and 31.

With continued reference to the various figures and now also to FIGS. 65 and 66, another alternative configuration is shown of a new and inventive cell and tissue culture vessel or device 830 that incorporates an extruded shell or body 832 having an anterior wall or face 834, a posterior wall or face 836, superior and inferior lateral peripheral edges 840, 842, dextral and sinistral longitudinal peripheral edges 844, 846, an interior chamber 850, and a plurality of lumens 852, 854, 856, 858, formed therein. Any of the preceding variations, modifications, and alternative arrangements of the cell and tissue culture flask 100 described herein are contemplated for incorporation into any of the extrusion-type modifications and alternative embodiments, and all such extruded features and elements are contemplated for incorporation into any of all of the precedingly illustrated non-extruded embodiments and modifications and alternative thereto. With reference now also to FIGS. 67 through 77 as well as 65 and 66, the alternatively extruded cell and tissue culture vessel 830 can be understood to also further incorporate a superior manifold 860 and an inferior manifold 862, which manifolds 860, 862 are adapted with respective inferior lateral edge 843 (superior manifold 860) and superior lateral edge 843′ (inferior manifold 862) for permanent or releasable receipt by and engagement with respective superior and inferior lateral body edges 840′, 842′. When assembled to the extruded shell or body 832, integrally formed lumens 852, 854, 856, 858 of the shell or body 832 are adapted to register with to communicate fluids (liquids and or gases) with corresponding superior and inferior manifold lumens and channels illustrated in the various figures and drawings and described further herein.

Using many of the same and similar principles of the features, elements, and components of the earlier described fluid transfer port 200, respirator 180, and the filtration and gas valvule 320, those knowledgeable in the related arts can comprehend that the extruded vessel 530 may incorporate a similarly or differently positioned and or otherwise modified fluid transfer port 870 and a similarly modified filtration and gas valvule 875, each of which can be adapted with any or all of the previously described features, elements, variations, and modifications illustrated in connection with the port 220 and the valvule 320. Further, one or more optional respirators 873 (FIG. 66) may be incorporated to have similar relative surface areas and features and capabilities as set forth with respect to the other embodiments, modifications, variations, and alternative configurations already described.

The port 870 and the filtration and gas valvule 875 can be formed in an interlocking and interchangeable manifold seat 845 that interlocks into the superior manifold 860 with any of a number of engagement features, which features can include, for purposes of illustration but not limitation, a wide range of effective slide-lock, snap-fit, bayonet and other types of joints including a dove-tail type joint fabricated of male and female sliding dove tail elements 845′ (FIGS. 75 and 76). Other equally suitable and for various applications perhaps preferable joint configurations can be formed with the latch and release features already described herein as well as various types of welds and adhesives for certain applications and can be constructed with joint configurations that can include, again for purposes of example without limitation, tongue and clevis, key and keyway, lap butt, half lap butt, mitered lap butt, gained (housed) butt, blind halved lap, mortise and tenon, miter half-lap butt, notched butt, plain dove tail butt, and dove tail half-lap joints and related features and elements. The interlocking and interchangeable manifold seat 845 can be adapted for slidable, releasable, and permanent receipt into the cistern 850 to register with apertures formed in the body 832 for the port 870 and valvule 875. A plurality of such interchangeable manifold seats are contemplated herein that can incorporate a range of configurations and capabilities of port 870 and valvule 875.

For further illustrative example, the instant fluid transfer port 870 may incorporate septum 872 and be adapted to communicate fluids into the chamber or cistern 850 via various lumens and or channels, such, for purposes of example without limitation, septum base lumen 856′ (FIG. 75), dextral lateral superior manifold lumen 857′ (FIG. 75), dextral longitudinal lumen 858 (FIGS. 68 and 69), dextral lateral inferior manifold lumen 858′ (FIGS. 70, 71, 72, and 74), and aspiration infusion exit port 859 (FIGS. 66, 68, and 72).

In yet more examples of suitable configurations according the principles of the instant invention, the filtration and gas valvule 875 preferably may incorporate one or more filter elements 874 adapted to have similar capabilities as filter and filtration elements 370, 380 to communicate substantially if not completely sterile gas between the external atmosphere and the interior cistern, chamber, or reservoir 850. Such sterile gas is communicated between the valvule 875 to the cistern 850 through a fluid labyrinth that can be similar in function that those embodiments described herein, including the siphon lock embodiments noted herein. As discussed elsewhere herein, such filtration elements 874 may be excluded in applications using modified pipetters and fluid transfer devices having sterile gas filtration elements incorporated therein.

Such a fluid labyrinth for the instant modified embodiments can accomplish the gas communication function between the cistern 850 and the external environment for pressure and vacuum relief via a variety of integrally formed lumens and channels, such as, for example without limitation, filtration seat lumen 852′, that be registered after assembly with one or more of the possibly desirable sinistral lateral superior manifold lumens 853′, that in turn communicate with one or more sinistral longitudinal lumens 852, 854, that communicate with one or more of sinistral lateral inferior manifold lumens 853, 855. The oft described siphon lock configuration is implemented in this modified construction of the preferred call and tissue culture flask or vessel 830 and in the context of the illustrated lumen configurations by any number of possible arrangements, including for further exemplary purposes, central inferior manifold channel 854′ (FIGS. 72 and 74) can be formed in center portion 844″ of inferior manifold 842 (FIGS. 72, 73, 74) that can be configured to communicate fluid between lumens 853 (FIGS. 72 and 74). In another possible alternative variation and with reference specifically to FIG. 73, a modified inferior manifold channel 855′ can be incorporated to communicate fluids between any one or more sinistral lateral inferior manifold lumens 853, 855 so as to effect the contemplated fluid labyrinth and or siphon lock capabilities already described in more detail herein. Additional fluid communication channels, such as channels 854′, 855′ can be formed in other portions of center portion 844″ to communicate fluids between dextral lateral inferior manifold lumens 858′, 858″ and or to any of the other lumens contemplated in the instant variations and alternative arrangements and as may be desirable for various labyrinth and siphon lock configurations.

The center portion 844″ may also include one or more joining feature similar to those described in connection with the interlocking and interchangeable manifold seat 845 that can also be formed to have the permanent or releasable capabilities of the male and female sliding dove tail elements 845′ already described. Such additional sliding, snap-fit, and or interlocking inferior dovetail elements 880 (male dove tail element on center portion 844″ (FIG. 72) of inferior manifold 862), 880′ (female dove tail elements of outboard segments of inferior manifold 862), 880″ (male dove tail elements of alternative channel 855″ arrangement of center portion 844″ in FIG. 73) may be formed as illustrated in FIGS. 72, 73, and 74 so as to join the various segments of inferior end manifold 862 to form the generally straight assembled configuration reflected in the figures. With continued reference to FIG. 72, dashed assembly chain line “L” describes the positioning of the elements of inferior end manifold 862 for assembly of the center portion 844″ along the direction of arrow R with the outboard segments of the manifold 862. With reference also now to FIG. 73 in combination with 72, dashed alternative assembly chain line L′ describes the positioning of the elements of inferior end manifold 862 for assembly of the modified center portion 844″, having the variation of alternative channel 855″, with the outboard segments of the manifold 862.

Various additional capabilities and features described hereinabove are also contemplated for use with the instant extrusion compatible flask or vessel 530 that can be included to permanently and releasably fasten together the superior and inferior end manifolds 860, 862 to the extruded shell or body 832. Such permanent and releasable fasteners and releasing features can include, for further purposes of explication but not for limitation, the welds, release notches, and other joint features and elements described in connection with any of the preceding preferred, optional, alternative, and modified embodiments and variations of the contemplated cell and tissue culture flask or vessels 100, 830. Such fastening and release features can be incorporated onto, into, and as integral parts the various extrusion compatible components in a variety of ways including modifying attachment rails 885, 888 (FIG. 75), 890 of the manifolds 860, 862 and center elements 840, 844″ and the receiving edge portions 840, 842′ of the extruded shell or body 832.

Turning next to yet more possibly desirably features and capabilities of the preferred and alternatively modified embodiments of the cell and tissue culture flasks or vessels 100, 830 according to the instant invention, reference is now made to FIGS. 78 through 84 wherein various forms of proposed identification and data storage features are illustrated in more detail.

In FIGS. 78 and 79, the cell and tissue culture flask 100, 830 can incorporate at least one and two or more, one, two, and multidimensional indicia and data encoding elements that are adapted to be encoded with and that can communicate (data can be read therefrom and in some configurations data can be written onto), and in some variations be modified and store, various data pertinent to use and operation of the flask or vessel 100, 830. With continued reference to FIG. 78, such one or single dimension indicia 900 can imprinted along any edge or side of the flask or vessel 100, 830 and can be in the form of a linear bar code identified as indicia 900 of the type that is commonly used on nearly every retail item displayed and offered for sale in the United States and that is also used in myriad product and inventory control systems including manufacturing and distribution, pharmacological dispensement, grocery, warehousing, logistics, and similar applications. Such barcode indicia can take many forms and can include, for purposes of example without limitation, encoding formats commonly known to those skilled in such technology as CodaBar, Code 25, Code 39, Code 128, EAN-8, EAN-13, ISBN, ISSN, ITF, ITF-14, JAN-8, JAN-13, MSI/Plessey, Pharmacode, UPC(A), and UPC(E), to name several such formats.

Also in FIG. 78, one or more two dimensional indicia such as target indicia 905 can also be employed in place of or in combination with other contemplated indicia and can be imprinted on any side or edge of the cell and tissue culture flask or vessel 100, 830. Such two or more dimensional indicia, which are similar in some or many respects to target indicia 905, are commonly employed in various present day applications, including package identification applications by carriers including the widely-known American companies United Parcel Service and Federal Express, among many other notable logistically sophisticated courier and carrier companies. Such multidimensional target indicia are also in wide use in various manufacturing, warehousing, logistics, and distribution businesses, which use such to identify, transport, and track large quantities of myriad inventory items.

Additionally, the instant invention also contemplates incorporation of a three or more dimensional indicia such as indicia element 910, shown in FIG. 78 with a portion removed to reveal a recess 915 that is formed in the flask or vessel 100, 830. The recess 915 can be sized and adapted to receive a portion of the indicia 910 that may incorporate other elements, including for purposes example without limitation, electronic data storage elements. The recess 915 may be wholly or partially covered by the indicia 910, which indicia 910 can be formed in a thin or thick film and or polymeric substrate embodiment similar in construction to the holographic type images used on present-day identification, debit, credit, and bank cards and or similar to the readable and, in some variations writable, optical, magneto-optical, and magnetic substrates used for music, video, and data compact discs and digital video discs and optical data cards. The indicia 910 may be formed to incorporate such additional electronically capable elements and or may cover the recess 915 wherein additional such components can be retained, which components can include a power source, an antenna, an induction coil, interface electronics that can communicate information received from various sensors incorporated into the flask or vessel 100, 830 to external devices, networks, and computers for purposes of monitoring the contents of and environmental conditions in and surrounding the flask or vessel 100, 830.

Further, the recess 915 can be formed into one or more faces, edges, or sides of the flask or vessel 100, 830 to accommodate such indicia 910 that may have a substantial thickness such that the indicia 910 can be recessed into recess 915 of the flask or vessel 100, 830 to maintain an upper surface of the indicia element 910 that is substantially flush and generally coplanar to that of the exterior face of flask or vessel 100, 830 as illustrated in FIG. 78. Those skilled in the art may also comprehend that magnetically readable and writable strip indicia 920 may be incorporated about any surface, side, or edge of the flask or vessel 100, 830 so as to enable identification and other data to be communicated to and from the magnetic strip indicia 920.

The recess 915 may be adapted to receive one or more electronic semiconductor chips or chipsets 925 similar in construction to the so-called present day “smart” cards in use by various financial institutions and security installations to identify the holder. Such chip or chipsets 925 may be employed either alone in recess 915 or in combination with the multidimensional indicia 910 and any other indicia contemplated herein. Such chips in such cards respond to communicate various data to a received device when the card in positioned proximate to the receiver. Instead of the “swiping” action presently also in use with the magnetic strips incorporated on many such cards, the smart cards only need to be placed in proximity to the reading receiver. The data transfer is accomplished in any number of ways and can be effected using induction type energy transfer methods, antenna and transmitter devices, and the like. In the context of the instant cell and tissue culture devices 100, 830 of the instant invention, one or more such smart chips or chipsets can be incorporated that can be adapted not only to be read by a received configured to communicate therewith, but chips or chipsets are contemplated for use herewith that can be adapted to record and store data relevant to the use and operation of the flask or vessel 100, 830. More particularly, for purposes of further example, in air sampling applications data such as position over time can be recorded so that the operator can define where a particular air sample or series of samples were obtained. In cell culture and related applications, time stamped data can include temperature, humidity, pressure, and various data that can enable the operator to ascertain whether the flask or vessel 100, 830 was subjected to anything other than optimum conditions during incubation.

With reference now also to FIG. 80, a cross-sectional view of a portion of the flask or vessel of FIG. 78 is depicted to illustrate additional details of the recess 915 and various arrangements of the indicia and chips chipsets. More specifically, the multidimensional indicia 910 is shown generally superior to and covering the recess 915 wherein the chips or chipset 925 are received. FIG. 81 depicts a schematic representation another possible configuration wherein the multi-dimensional indicia 910 is includes magnetic, optical and or magneto-optical layers 912 that are further adapted as, for example without limitation, what is customarily known to those having skill in the art as a magnetic, an optical, a magneto-optical, and or similarly capable substrate that can be read by and be written to optical read/write sensors S and magnetic read/write sensors S′. With continued reference to the preceding figures and illustrations, FIGS. 82, 83, and 84 are top or plan views of the various arrangements of the target indicia 905, the multi-dimensional indicia 910, and the chips and or chipsets 925 configurations.

Turning next to FIGS. 85 through 90, the cell and tissue culture vessel or flask 100, 830 is illustrated in combination with a protective holder and insulating device, which can be further adapted to incorporate various components that can establish the capability for an incubation and cooling container apparatus 1000. The container apparatus 1000 can be formed from an opaque, translucent, or transparent material, and combinations thereof, and preferably defines a flask cavity 1010 when a receiver 1020 is mated with a cover 1030 of the container 1000 that together define a flask cavity 1030 that is received with the flask 100, 830. Using any of a variety of technologies, the flask or vessel 100, 830 is insulated from exterior ambient temperatures and can in various constructions of the container 1000 be protected from shock and impact loads during incubation, storage, handling, and transportation.

In FIGS. 88, 89, and 90, those skilled in the art can understand that the incubation and cooling container can be formed from, among many other possible constructions, a polymeric polyisoprene or natural or synthetic rubberized material, a thermosetting foam such as a urethane, a styrofoam, and other similarly capable shock absorbing and heat transfer insulating material 1040. Further, an outside polymeric shell 1045 of the container 1000 can be incorporated that is preferably formed from an impact resistance material that can include, among many types of metals, ceramics, and plastics, and polymeric materials such as high-strength polycarbonates and ABS thermoplastics.

With reference specifically also to FIG. 89, wherein the insulating material 1040 and outside shell 1045 materials has been removed for purposes of further explication, the container 1000 also incorporates a power source 1050 such as an AC-based interconnection, induction coil energy transfer device, or one or more batteries 1052. The power source 1050 is in electronic communication with control electronics 1055 that are also connected to a control switch or switches 1057 that can be configured to actuate the container 1000 for cooling or heating/incubation operation, and which can be further adapted in various configurations to control temperature and other contemplated capabilities of the container 1000. A thermoelectric device 1060 is also in electronic communication with the control electronics 1055 and is preferably thermally separated from the control electronics 1055 by an insulator 1062, which can be further configured as a radiator coupled to radiator plates 1064. The thermoelectric device 1060, which is also known to those skilled in the relevant arts as a Peltier device, pumps heat across a semiconductor substrate when subjected to a voltage and current. The device 1060 is also coupled thermally to conductor plates 1070, which are positioned about the interior walls that define the cavity 1010 within the receiver 1030 of the container 1000. Any of a number of types of thermal sensing devices, such as thermistors 1080 can be incorporated and placed in electronic communication with the control electronics 1055 and in thermal contact with the conductor plates 1070 whereby the control electronics can adjust the voltage and current communicated to the thermoelectric device 1060 to maintain the desired warming or cooling temperature of the conductor plates 1070. In FIGS. 91 and 92, an alternative configuration of an incubation and cooling container 1100 is shown wherein more than one cell and tissue culture device 100, 830 can be received for cooled or heated incubation, storage, handling, and transportation. The modified container 1100 is adapted with a plurality of conductor plates 170 that, in combination with the interior walls of modified receiver 1030′, form respective flask cavities 1010′. Although not shown in the various figures, the instant configurations and embodiments of the proposed containers 1000 and 1100 are further susceptible to alternative arrangements and configurations that incorporate alternative heating, cooling, power source modifications that can be compatible for use with induction coupled energy sources. For example, various arrangements of microwave transmitters can be employed for purposes of maintaining the most desired incubation temperatures for purposes of incubating cell cultures contained in the flask or vessel 100, 830. Similarly, various types of other frequency and amplitude modulated and similar electromotive energy transfer arrangements that employ very low (for example, infrared and radio) to super and ultra high frequencies (microwaves, and or ordinary and millimeter wavelength radar frequencies) can be effective for maintaining such desired incubation temperatures. Moreover, using any of the smart card, induction coil, and other types of data communication capabilities contemplated herein, such external power and energy sources can be regulated to input the needed energy to the myriad possible embodiments of the cell and tissue culture devices 100, 830 illustrated herein and contemplated hereby. Although the various descriptions herein are directed primarily to temperature sensors in the containers 1000 and 1100, the flask and vessel 100, 830 can also be further adapted in alternative configurations to incorporate sensors onto, into, and integral with the shells, walls, and or body 110, 140, 832, which sensors can include data acquisition sensors that measure, store, and communicate temperature as well as, for purposes of example but not limitation, pH (alkalinity and acidity), pressure, viscosity, radiation absorbance (light wavelengths and other wavelengths that can identify certain parameters of the culture), emittance, opacity, and the like.

Numerous alterations, modifications, and variations of the preferred embodiments disclosed herein would be apparent to those skilled in the art and they are all contemplated to be within the spirit and scope of the instant invention, which is limited only by the following claims. For example, although specific embodiments have been described in detail, those with skill in the art can understand that the preceding embodiments and variations can be modified to incorporate various types of substitute and/or additional materials, relative arrangement of elements, and dimensional configurations for compatibility with the wide variety of possible garments that are available in the marketplace. Accordingly, even though only few embodiments, alternatives, variations, and modifications of the present invention are described herein, it is to be understood that the practice of such additional modifications and variations and the equivalents thereof, are within the spirit and scope of the invention as defined in the following claims.

I claim:

1. A cell culture device, comprising:
generally planar anterior and posterior shells arranged in a confronting relationship and joined by respective opposing dextral and sinistral longitudinal peripheral edges, and opposing superior and inferior peripheral lateral edges, the shells and edges having a surface area defining a media reservoir, at least one of the anterior and posterior shells and edges being formed with at least one circumfluent periphery and defining at least one aperture;
at least one gas permeable membrane sealing the at least one aperture and joined to the periphery;
at least one fluid transfer port formed in least one of the shells and edges and in fluid communication with the media reservoir; and
at least one gas valvule formed in at least one of the shells and edges and in fluid communication with the media reservoir.

2. The cell according to claim 1, wherein the surface area of the membrane is approximately between 1% and 10% of the surface area of the media reservoir.

3. The cell culture device according to claim 1, wherein the surface area of the membrane is approximately between 1.5% and 5% of the surface area of the media reservoir.

4. The cell culture device according to claim 1, wherein the at least one gas valvule is hydrophobic and adapted to prevent liquid flow therethrough.

5. The cell culture device according to claim 1, wherein the media reservoir is adapted to receive approximately between 20 milliliters and 140 milliliters of a fluid mixture.

6. The cell culture device according to claim 1, wherein the media reservoir is adapted to receive at least about 25 milliliters of a fluid mixture.

7. The cell culture device according to claim 1, wherein the joint formed between the respective lateral, superior, and inferior peripheral edges is formed as releasably hermetically sealed joint.

8. The cell culture device according to claim 1, wherein the media reservoir is formed to have a lateral dimension between the laterally opposed longitudinal edges of approximately between 6.5 centimeters and 9 centimeters, a longitudinal dimension between opposing superior and inferior lateral edges of approximately between 11 and 13 centimeters, and a dimension between interior surfaces of the anterior and posterior shells of approximately between 2 millimeters and 6 millimeters.

9. The cell culture device according to claim 1, wherein at least one of the anterior and posterior shells are formed from a thermoplastic material that is substantially transparent.

10. The cell culture device according to claim 9, wherein at least one of the shells is formed from a thermoplastic material to have a pigment adapted to filter photonic energy outside the range of between approximately 500 and 600 nanometers.

11. The cell culture device according to claim 9, wherein at least one of the shells is formed from a thermoplastic material having a pigment adapted to filter photonic energy outside the range of between approximately 550 and 570 nanometers.

12. The cell culture device according to claim 1, wherein the at least one gas permeable membrane is formed from a sheet material to have a thickness approximately between 0.09 and 0.14 millimeters.

13. The cell culture device according to claim 1, wherein the media reservoir defines in internal surface area and the at least one gas permeable membrane has a second surface area approximately between 1.5% and 10% of the internal surface area.

14. The cell culture device according to claim 1, wherein the at least one fluid transfer port communicates fluid with the media reservoir through a siphon lock lumen formed with at least one fluid path that bends through at least one angle of approximately between 45 and 135 degrees of arc.

15. The cell culture device according to claim 1, wherein the at least one fluid transfer port incorporates a resealable elastomeric septum adapted to releasably receive a means to communicate a fluid through the port.

16. The cell culture device according to claim 1, wherein the at least one gas valvule communicates gas with the media reservoir through a second siphon lock lumen formed with at least one fluid path that bends through at least one angle of approximately between 45 and 135 degrees of arc.

17. The cell culture device according to claim 1, wherein the at least one gas valvule incorporates a filtration element adapted to pass gaseous molecules and to prevent the passage of particles having an average diametrical dimension of approximately between 0.1 and 0.3 microns.

18. The cell culture device according to claim 17, wherein the filtration element incorporates a hydrophobic material.

19. The cell culture device according to claim 17, wherein the filtration element is an assembly of at least 2 layers with a first layer being adapted to prevent the passage of particles having an average diametral dimension of at least between about 80 to 120 microns, and a second layer being adapted to prevent the passage of particles having an average diametral dimension of at least between about 0.1 to 0.3 microns.

20. The cell culture device according to claim 17, wherein the filtration element is a hybrid filter medium having a filtration property wherein the size of the particles that are filtered changes across a cross-section of the filter medium such that at a first exterior surface the medium is adapted to prevent the passage of particles having an average diametral dimension of at least between about 80 to 120 microns, and whereby at a second opposite exterior surface the medium is adapted to prevent the passage of particles having an average diametral dimension of at least between about 0.1 to 0.3 microns.

21. The cell culture device according to claim 1, wherein the media reservoir is defined by an internal surface area of the shells and edges that bounds an internal volume whereby the ratio between the volume and the surface area is approximately between 100 microliters per square centimeter and 1000 microliters per square centimeter.

22. The cell culture device according to claim 1, wherein the media reservoir is defined by a plurality of internal surfaces of the shells and edges wherein substantially all of the surfaces are adapted to support growth of cells.

23. A cell culture device, comprising:
a reservoir formed from generally transparent confronting anterior and posterior walls joined about respectively opposed superior and inferior peripheral lateral edges, and respective laterally opposed peripheral longitudinal edges, the walls and edges defining an interior cistern;
at least one aperture formed in a least one of the walls and edges and sealed with a gas permeable membrane and in gaseous communication with the cistern;
at least one injection and aspiration port formed in at least one of the walls and edges and in fluid communication with the cistern; and
at least one pressure relief valvule formed in at least one of the walls and edges and in gaseous communication with the cistern, the valvule being operative to equalize pressure within the cistern to ambient atmospheric pressure as fluid is communicated through the at least one port.

24. The cell culture device according to claim 23, wherein the surface area of the membrane is approximately between 1% and 10% of the surface area of the cistern.

25. The cell culture device according to claim 23, wherein the surface area of the membrane is approximately between 1.5% and 5% of the surface area of the cistern.

26. The cell culture device according to claim 23, wherein the at least one valvule is hydrophobic and adapted to prevent fluid flow therethrough.

27. The cell culture device according to claim 23, wherein the cistern is adapted to receive approximately between 20 milliliters and 140 milliliters of a fluid mixture.

28. The cell culture device according to claim 23, wherein the cistern is adapted to receive at least about 25 milliliters of a fluid mixture.

29. The cell culture device according to claim 23, wherein the joint formed between the respective lateral, superior, and inferior peripheral edges is formed as releasably hermetically sealed joint.

30. The cell culture device according to claim 23, wherein the media reservoir is formed to have a lateral dimension between the opposing longitudinal edges of at least approximately between 6 centimeters and 9 centimeters, a longitudinal dimension between opposing superior and inferior lateral edges of at least approximately between 11 and 13 centimeters, and a dimension between interior surfaces of the anterior and posterior shells of at least approximately between 2 millimeters and 6 millimeters.

31. The cell culture device according to claim 23, wherein at least one of the anterior and posterior shells are formed from a thermoplastic material that is substantially transparent.

32. The cell culture device according to claim 31, wherein at least one of the shells is formed from a thermoplastic material to have a pigment adapted to filter photonic energy outside the range of between approximately 500 and 600 nanometers.

33. The cell culture device according to claim 31, wherein at least one of the shells is formed from a thermoplastic material having a pigment adapted to filter photonic energy outside the range of between approximately 550 and 570 nanometers.

34. The cell culture device according to claim 23, wherein the at least one gas permeable membrane is formed from a sheet material to have a thickness approximately between 0.09 and 0.14 millimeters.

35. The cell culture device according to claim 23, wherein the cistern defines in internal surface area and the at least one gas permeable membrane has a second surface area approximately between 1.5% and 10% of the internal surface area.

36. The cell culture device according to claim 23, wherein the at least one injection and aspiration port communicates fluid with the cistern through a lumen formed with at least one fluid path that bends through at least one angle of approximately between 45 and 135 degrees of arc.

37. The cell culture device according to claim 23, wherein the at least one injection and aspiration port incorporates a resealable elastomeric septum adapted to releasably receive a means to communicate a fluid through the port.

38. The cell culture device according to claim 23, wherein the at least one pressure relief valvule communicates gas with the cistern through a second lumen formed with at least one fluid path that bends through at least one angle of approximately between 45 and 135 degrees of arc.

39. The cell culture device according to claim 23, wherein the at least one pressure relief valvule incorporates a filtration element adapted to pass gas and to inhibit liquid flow and to prevent the passage of particles having an average diametrical dimension of approximately between 0.1 and 0.3 microns.

40. The cell culture device according to claim 39, wherein the filtration element incorporates a hydrophobic material.

41. The cell culture device according to claim 39, wherein the filtration element is an assembly of at least 2 layers with a first layer being adapted to prevent the passage of particles having an average diametral dimension of at least between about 80 to 120 microns, and a second layer being adapted to prevent the passage of particles having an average diametral dimension of at least between about 0.1 to 0.3 microns.

42. The cell culture device according to claim 39, wherein the filtration element is a hybrid filter medium having a filtration property wherein the size of the particles that are filtered changes across a cross-section of the filter medium such that at a first exterior surface the medium is adapted to prevent the passage of particles having an average diametral dimension of at least between about 80 to 120 microns, and whereby at a second opposite exterior surface the medium is adapted to prevent the passage of particles having an average diametral dimension of at least between about 0.1 to 0.3 microns.

43. The cell culture device according to claim 23, wherein the cistern is defined by an internal surface area of the wall and edges that bounds an internal volume whereby the ratio between the volume and the surface area is approximately between 100 microliters per square centimeter and 1000 microliters per square centimeter.

44. The cell culture device according to claim 23, wherein the cistern is defined by a plurality of internal surfaces of the walls and edges wherein substantially all of the surfaces are adapted to support growth of cells.

45. A cell culture device, comprising:
a reservoir formed from generally transparent confronting anterior and posterior walls joined about respectively longitudinally opposed superior and inferior peripheral lateral edges, and respective laterally opposed peripheral longitudinal edges, the walls and edges defining an interior cistern;
at least one injection and aspiration port formed in at least one of the walls and edges and in fluid communication with the cistern; and
at least one pressure relief valvule formed in at least one of the walls and edges and in gaseous communication with the cistern, the valvule being operative to equalize pressure within the cistern to ambient atmospheric pressure as fluid is communicated through the at least one port.

46. The cell culture device according to claim 45, wherein the at least one valvule is hydrophobic and adapted to prevent fluid flow therethrough.

47. The cell culture device according to claim 45, wherein the cistern is adapted to receive approximately between 20 milliliters and 140 milliliters of a fluid mixture.

48. The cell culture device according to claim 45, wherein the cistern is adapted to receive at least about 25 milliliters of a fluid mixture.

49. The cell culture device according to claim 45, wherein the joint formed between the respective lateral, superior, and inferior peripheral edges is formed as releasably hermetically scaled joint.

50. The cell culture device according to claim 45, wherein the media reservoir is formed to have a lateral dimension between the opposing lateral edges of at least approximately between 6 centimeters and 9 centimeters, a longitudinal dimension between opposing superior and inferior edges of at least approximately between 11 and 13 centimeters, and a dimension between interior surfaces of the anterior and posterior shells of at least approximately between 2 millimeters and 6 millimeters.

51. The cell culture device according to claim 45, wherein at least one of the anterior and posterior shells are formed from a thermoplastic material that is substantially transparent.

52. The cell culture device according to claim 51, wherein at least one of the shells is formed from a thermoplastic material to have a pigment adapted to filter photonic energy outside the range of between approximately 500 and 600 nanometers.

53. The cell culture device according to claim 51, wherein at least one of the shells is formed from a thermoplastic material having a pigment adapted to filter photonic energy outside the range of between approximately 550 and 570 nanometers.

54. The cell culture device according to claim 45, wherein the at least one gas permeable membrane is formed from a sheet material to have a thickness approximately between 0.09 and 0.14 millimeters.

55. The cell culture device according to claim 45, wherein the at least one injection and aspiration port communicates fluid with the cistern through a lumen formed with at least one fluid path that bends through at least one angle of approximately between 45 and 135 degrees of arc.

56. The cell culture device according to claim 45, wherein the at least one injection and aspiration port incorporates a resealable elastomeric septum adapted to releasably receive a means to communicate a fluid through the port.

57. The cell culture device according to claim 45, wherein the at least one pressure relief valvule communicates gas with the cistern through a second lumen formed with at least one fluid path that bends through at least one angle of approximately between 45 and 135 degrees of arc.

58. The cell culture device according to claim 45, wherein the at least one pressure relief valvule incorporates a filtration element adapted to pass gas and to inhibit liquid flow and to prevent the passage of particles having an average diametrical dimension of approximately between 0.1 and 0.3 microns.

59. The cell culture device according to claim 58, wherein the filtration element incorporates a hydrophobic material.

60. The cell culture device according to claim 58, wherein the filtration element is an assembly of at least 2 layers with a first layer being adapted to prevent the passage of particles having an average diametral dimension of at least between about 80 to 120 microns, and a second layer being adapted to prevent the passage of particles having an average diametral dimension of at least between about 0.1 to 0.3 microns.

61. The cell culture device according to claim 58, wherein the filtration element is a hybrid filter medium having a filtration property wherein the size of the particles that are filtered changes across a cross-section of the filter medium such that at a first exterior surface the medium is adapted to prevent the passage of particles having an average diametral dimension of at least between about 80 to 120 microns, and whereby at a second opposite exterior surface the medium is adapted to prevent the passage of particles having an average diametral dimension of at least between about 0.1 to 0.3 microns.

62. The cell culture device according to claim 45, wherein the cistern is defined by an internal surface area of the wall and edges that bounds an internal volume whereby the ratio between the volume and the surface area is approximately between 100 microliters per square centimeter and 1000 microliters per square centimeter.

63. The cell culture device according to claim 45, wherein the cistern is defined by a plurality of internal surfaces of the walls and edges wherein substantially all of the surfaces are adapted to support growth of cells.

64. The cell culture device according to claim 45, wherein the anterior and posterior walls and the laterally opposed peripheral longitudinal edges form a body having a superior body edge and an inferior body edge, and wherein the superior and inferior peripheral lateral edges are further formed on respective superior and inferior end manifolds adapted to be respectively engaged with the superior and inferior body edges to further define the cistern

65. The cell culture device according to claim 45, further comprising:
an insulative and protective container defining at least one interior cavity for receiving at least one cell culture device and wherein the container incorporates a means for controlling the temperature of the device.
